(19) United States
(12) Patent Application Publication
(10) Pub. No.: US 2021/0388012 A1
(43) Pub. Date: Dec. 16, 2021

(54) METHODS AND COMPOSITIONS FOR PRODRUG FORMS OF SPECTINOMYCIN AND SPECTINAMIDE ANALOGS

(71) Applicant: ST. JUDE CHILDREN'S RESEARCH HOSPITAL, INC., Memphis, TN (US)

(72) Inventors: Richard E. LEE, Cordova, TN (US); Jiuyu LIU, Collierville, TN (US)

(21) Appl. No.: 17/288,493

(22) PCT Filed: Nov. 5, 2018
(86) PCT No.: PCT/US2019/059848

§ 371 Date: Apr. 23, 2021

Related U.S. Application Data
(60) Provisional application No. 62/755,765, filed on Nov. 5, 2018.

(51) Int. Cl. C07F 9/6561; C07D 493/04; A61K 45/06

(52) U.S. Cl. CPC C07F 9/6561; A61K 45/06; C07D 493/04

(57) ABSTRACT

In one aspect, the disclosure relates to substituted spectinomycin analogs, including substituted aminomethyl spectinomycin analogs and substituted spectinamide analogs, with increased tolerability and safety, including improved tolerability to parenteral administration. The present disclosure further pertains to methods of making disclosed compounds, pharmaceutical compositions comprising the disclosed compounds, and methods of treating microbial infections using the disclosed compounds, including methods of treating antibiotic resistant infections and tuberculosis. This abstract is intended as a scanning tool for pur-poses of searching in the particular art and is not intended to be limiting of the present disclosure.

### CROSS-REFERENCE TO RELATED APPLICATIONS

This Application claims the benefit of U.S. Provisional Application No. 62/755,765, filed on Nov. 5, 2018, which is incorporated herein by reference in its entirety. portion-level

This invention was made with government support under grant number AI090810 awarded by the National Institute of Health. The governmenht has certain rights in the invention.

### BACKGROUND

The dramatic rise in the prevalence of antibiotic resistance among bacteria requires the discovery and development of new antimicrobials to treat infections caused by these organisms. Of major health concern are drug resistant infections caused by Methicillin-resistant Staphylococcus aureus (MRSA), vancomycin-resistant Enterococcus, multidrug resistant Streptococci pneumoniae, Neisseria gonorrhoeae, and and Mycobacterium tuberculosis, pan-resistant Klebsiella pneumoniae, Pseudomonas aeruginosa and Acinetobacter baumannii (Fischbach et al. Science 2009, 325 (5944), 1089-1093; Goldston et al. Suicide Life Threat. Behav, 2010, 40(3), 245-256; Nicasio et al, Pharmacotherapy 2008, 28 (2), 235-249). There is also a need for new therapeutic agents to treat biodefense pathogens.

Mycobacterium tuberculosis, the causative agent of tuberculosis, remains one of the world's most deadly infectious diseases. It is estimated that tuberculosis (TB) is the leading cause of death from infectious disease in the world. As of 2007, the World Health Organization estimated that more than three million active cases of TB occurred worldwide annually leading to greater than one million deaths (e.g., see World Health Organization, WHO Report 2007). HIV infected individuals are more prone to become infected with and develop the active form of TB, and as the HIV pandemic has spread across the globe this has significantly contributed to the increase in the number of TB cases observed globally. See Centers for Disease Control, TB and HIV Coinfection, 2006.

The currrently recommend treatment for TB is a four drug regime for a minimum of six months that includes rifampin, isoniazid, pyrazinamide and ethambutol; This lengthy and burdensome regime leads to non-compliance by patients. This in turn has produced an increasing number of multidrug resistant (MDR) and extensively drug resistant (XDR) strains found in the clinic, for which effective therapeutic options are severely limited. Multidrug-resistant TB (MDR-TB) and and extensively drug-resistant (XDR-TB) are challenging infections for a clinician to treat. and they remain a significant public health threat (e.g., see D. T. Hoagland, J. Lira, R. B. Lee and R. E. Lee, Adv Drug Deliv Rev, 2016, 102, 55-72). For example, MDR/XDR TB can take two years or more to treat with drugs that are less potent, more toxic and much more expensive than the drugs used to treat non-drug resistant TB (e.g., M. D. Iseman, New Eng J Med, 1993_{;} 329, 1435-1435).

Spectinomycin is an aminocyclitol antibiotic that specifically inhibits bacterial protein synthesis by binding to 30S ribosome at a unique site that is highly conserved across bacterial pathogens (Carter et al. Nature 2000, 407(6802), 340-348; Borovinskaya et al. ACS Chem. Biol. 2007, 2 (8), 545-552; Wirmer et al. Meth. Enzmol. 2006, 415, 180-202). Although spectinomycin is potent in cell free assays its clinical use is restricted to second line treatment for Neisseria gonorrhoeae infections (McCormack et al. Annals of internal medicine 1976, 84 (6), 712-716; Reyn et al. Br. J. Verger. Dis. 1973, 49 (1), 54-59; Zenilman et al. J. Infect. Dis. 1987, 156 (6), 1002-1004. Over 25 years ago, attempts to develop spectinomycin analogs led to the discovery of trospectinomycin, which progressed into late stage clinical trials before being abandoned by Upjohn.

Spectinamides are semisynthetic spectinomycin analogs with selective ribosomal inhibition and excellent anti-tubercular activity (Nat Med. 2014 February; 20(2): 152-158). In multiple murine infection models, these spectinamides were well tolerated, significantly reduced lung mycobacterial burden and increased survival (Nat Med. 2014 February; 20(2): 152-158). It has been reported that a key to their potent antitubercular properties was their structural modification to evade the Rv1258c efflux pump, which is upregulated in MDR strains.

Despite advances in research directed to safe and efficacious treatments for new and improved antimicrobial treatments, including those directed to antibiotic resistant microbes and tuberculosis infections, including MDR-TB and XDR-RB, there is still a scarcity of compounds that are both potent, efficacious, and safe therapeutic agents. Moreover, despite the encouraging results obtained with both substituted spectinomycin analogs, including spectinamide derivatives. there remains a need for compounds based on these pharmacophores that have improved tolerability and safety. In particular, there remains a need for spectinomycin and spectinamide derivatives that are well-tolerated for parenteral administration. These needs and other needs are satisfied by the present disclosure.

### SUMMARY

In accordance with the purpose(s) of the disclosure, as embodied and broadly described herein, the disclosure, in one aspect, relates to substituted spectinomycin analogs, including substituted aminomethyl spectinomycin analogs and substituted spectinamide analogs, with increased tolerability and safety, including improved tolerability to parenteral administration. The present disclosure further pertains to methods of making disclosed compounds, pharmaceutical compositions comprising the disclosed compounds, and methods of treating microbial infections using the disclosed compounds, including methods of treating antibiotic resistant infections and tuberculosis.

Disclosed are compounds having a structure represented by a formula:

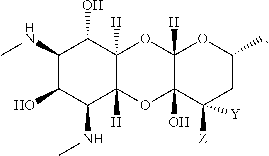

wherein Y is hydrogen or hydroxyl; wherein Z is —CH₂—NH—(C1-C3 alkanediyl)Ar¹ or —NH—(C═O)—(C1-C3 alkanediyl)-Ar¹; wherein Ar¹ is aryl or heteroaryl substituted with: (a) a R²⁰ group, wherein R²⁰ is selected from (C1-C3)-alkanediyl-OP(O)(OR²¹) (OR²²), —OP(O)(OR²¹)(OR²²), —(C1-C3)-alkanediyl-OSO₂OR²¹ , —OSO₂OR²¹, —(C1-C3)-alkanediyl-OSO₂R²¹, —OSO₂R²¹, (C1-C3)-alkanediyl-OSO₂NR²¹R²², —OSO₂NR²¹R²², wherein each of R²¹ and R²² is independently selected from hydrogen and C1-C3 alkyl; and (b) Cto 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

Also disclosed are compounds having a structure represented by a formula:

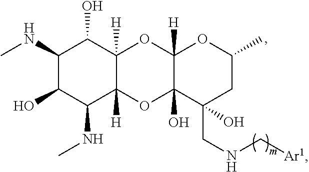

wherein m is an integer selected from 0, 1, 2, and 3; wherein Ar¹ is aryl or heteroaryl substituted with: (a) a R²⁰ group, wherein R²⁰ is selected from —(C1-C3)-alkanediyl-OP(O)(OR²¹)(OR²²), —OP(O)(OR²¹)(OR²²), —(C1-C3)-alkanediyl-OSO₂OR²¹, —OSO₂OR²¹, —(C1-C3)-alkanediyl-OSO₂R²¹, —OSO₂R²¹, (C1-C3)-alkanediyl-OSO₂NR²¹R²², —OSO₂NR²¹R²², wherein each of R²¹ and R²² is independently selected from hydrogen and C1-C3 alkyl; and (b) Cto 2 groups independently selected from halo, cyano, hydroxyl, —H₂, C1-C3 alkyl, C1-C3 haloalkyl. C1-C3 alkoxy, and C1-C3 haloalkoxy; or a pharmaceutically acceptable salt thereof.

Also disclosed are compounds having a structure represented by a formula:

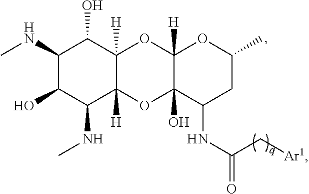

wherein q is an integer selected from 0, 1, 2, and 3; wherein Ar¹ is aryl or heteroaryl substituted with: (a) a R²⁰ group, wherein R²⁰ is selected from —(C1-C3)-alkanediyl-OP(O)(OR²¹) (OR²²), —OP(O)(OR²¹)(OR²²), —(C1-C3)-alkanediyl-OSO₂OR²¹, —OSO₂OR²¹, —(C1-C3)-alkanediyl-OSO₂R²¹, —OSO₂R²¹, —(C1-C3)-alkanediyl-OSO₂NR²¹R₂₂, —OSO₂NR²¹R²², wherein each of R²¹ and R²² is independently selected from hydrogen and C1-C3 alkyl; and (b) Cto 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

Also disclosed are pharmaceutical compositions comprising a therapeutically effective amount of a disclosed compound, or a pharmaceutically acceptable salt thereof, and a pharmaceutically acceptable carrier.

Also disclosed are methods for treating an infectious disease in a human subject comprising the step of administering to the human subject a therapeutically effective amount of at least one of the disclosed compounds, or a pharmaceutically acceptable salt thereof.

Also disclosed are methods for treating an infectious disease in a vertebrate animal comprising the step of administering to the vertebrate animal a therapeutically effective amount of at least one of the disclosed compounds, or a pharmaceutically acceptable salt thereof.

Also disclosed are methods for treating a human subject for a disorder associated with exposure to a biodefense pathogen comprising the step of administering to the human subject an effective amount of at least one of the disclosed compounds, or a pharmaceutically acceptable salt thereof.

Also disclosed are methods for inhibiting protein synthesis in at least one bacterial cell, comprising the step of contacting the bacterial cell with an effective amount of at least one disclosed compound, or a pharmaceutically acceptable salt thereof.

Also disclosed are uses of a disclosed compound, or a pharmaceutically acceptable salt thereof; a disclosed product of making, or a pharmaceutically acceptable salt thereof; or a disclosed pharmaceutical composition.

Also disclosed are uses of a disclosed compound, or a pharmaceutically acceptable salt thereof, in the manufacture of a medicament with a pharmaceutically acceptable carrier or diluent for the treatment of a disorder associated with a microbial infection in a mammal,

Also disclosed are methods for the manufacture of a medicament to inhibit protein synthesis in a microbe infecting a mammal comprising combining at least one disclosed compound, or a pharmaceutically acceptable salt thereof in the manufacture of a medicament with a pharmaceutically acceptable carrier or diluent.

Also disclosed are kits comprising at least one disclosed compound, or a pharmaceutically acceptable salt thereof; or a disclosed pharmaceutical composition; and one or more of: (a) at least one agent known to microbial ribosomal activity; (b) at least one agent known to have antimicrobial activity; (c) at least one agent known to treat an infectious disease; (d) instructions for treating an infectious disease; (e) instructions for administering the compound in connection with treating a microbial infection; or (f) instructions for administering the compound with at least one agent known to treat an infectious disease.

While aspects of the present disclosure can be described and claimed in a particular statutory class, such as the system statutory class, this Is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the disclosure.

FIG. 1 shows representative data comparing efficacy of a representative disclosed compound, 3408, in a mouse model of acute tuberculosis infection as described in the Examples. The data compare dosing as follows: (a) untreated control; (b) treatment with compound 1810 (subcutaneous injection, 400 mg/kg); (c) treatment with disclosed compound 3408 (subcutaneous injection, 400 mg/kg); and (d) treatment with rifampicin (oral gavage, 10 mg/kg).

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art, Such techniques are explained fully in the literature.

Prior to describing the various aspects of the present disclosure, the following definitions are provided and should be used unless otherwise indicated. Additional terms may be defined elsewhere in the present disclosure.

### A. Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially” or and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about” 10′ is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to as well as the range greater than ‘x’ and less than ‘y’. The range can also be expressed as an upper limit, e.g. ‘about x, y, z, or less’ and should be interpreted to include the specific ranges of ‘about x’, ‘about’ and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about C.1% to 5%” should be interpreted to include not only the explicitly recited values of about C.1% to about 5%, but also include individual values (e.g., about 1%, about 2%, about 3%, and about 4%) and the sub-ranges (e.g., about C.5% to about 1.1%; about 5% to about 2.4%; about C.5% to about 3.2%, and about C.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within ±10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, “spectinomycin” and “SPC” can be used interchangeably, and refer to a compound having a structure represented by a formula:

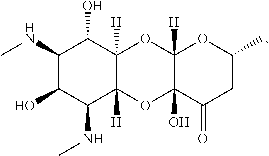

and is an antibiotic produced by Streptomyces spectabilis. Alternatively, spectinomycin can also be referred to as (2R,4aR,5aR,6S,7S,8R,9S,9aR,10aS)-4a,7,9-trihydroxy-2-methyl-6,8-bis(methylamino)decahydro-4H-1-benzo[b]pyrano[2,3-e][1,4]dioxin-4-one, Actinospectacin, Trobicin, Togamycin, Spectam, espectinomicina, spectinomycine, spectinomicina, and actinospectacina. It is understood that the core spectinomycin tricyclic ring structure has the following numbering convention:

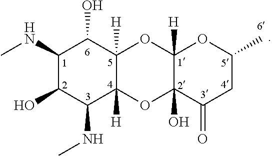

The term “bacteria” as used herein is intended to encompass all variants of bacteria, for example, prokaryotic organisms and cyanobacteria. Accordingly, bacterial infections to be treated using the compositions and methods described herein include, but are not limited to, infections caused by gram-positive bacteria such as, but not limited to, Bacillus cereus, Bacillus anthracis, Bacillus cereus, Bacillus anthracis, Clostridium botulinum, Clostridium difficle, Clostridium tetani, Clostridium perfringens, Corynebacteria diptheriae, Enterococcus (Streptococcus D), Listeria monocytogenes, Pneumococcal infections (Streptococcus pneumoniae), Staphylococcal infections and Streptococcal infections; infections caused by gram-negative bacteria such as, but not limited to, Bacteroides sp., Bordetella pertussis, Brucella sp., Chlamydia trachomatis, Chlamydia sp., Campylobacter infections, enterohaemorrhagic Escherichia coli (EHEC/E. coli C157:17), enteroinvasive Escherichia coli (ElEC), enterotoxigenic Escherichia coli (ETEC), Haemophilus influenzae, Helicobacter pylori, Klebsiella pneumoniae, Legionella sp., Moraxella catarrhalis, Neisseria gonnorrhoeae, Neisseria meningitidis, Proteus sp., Pseudomonas aeruginosa, Salmonella sp., Shigella sp., Vibrio cholera and Yersinia; infections caused by acid fast bacteria including, but not limited to, Mycobacterium tuberculosis, Mycobacterium avium-intracellulare, Mycobacterium johnei, Mycobacterium leprae, atypical bacteria, Mycoplasma, Rickettsia, Spirochetes, Treponema pallidum, Borrelia recurrentis, Borrelia burgdorfii and Leptospira icterohernorrhagiae and other miscellaneous bacteria, including Actinomyces sp. and Nocardia sp.

Examples of bacterial infections and situations in which such bacterial infections can occur that are not necessarily specific to a particular bacterial species, but encompassed by the term “bacterial infection,” as used herein, include bacterial wound infections, such as in burn wound patients; mucosal infections, enteric infections, bacteremia and septic conditions, pneumonia, trachoma, onithosis, trichomoniasis and salmonellosis, especially in veterinary practice; urinary tract infections; post-surgery infections on or caused by invasive devises; endocarditis by intravenous administration of contaminated drug solutions; bacterial infections in patients with acquired immunodeficiency syndrome, cancer chemotherapy, steroid therapy, hematological malignancies, organ transplantation, renal replacement therapy, and other situations with severe neutropenia; community-acquired respiratory tract infections; meningitis; folliculitis and infections of the ear canal caused by contaminated waters; malignant otitis externa in the elderly and diabetics; osteomyelitis of the caleaneus in children; eye infections commonly associated with contaminated contact lens; Skin infections such as nail infections in people whose hands are frequently exposed to water; gastrointestinal tract infections; and musculoskeletal system infections.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes; (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g cattle, horses, pigs, sheep, goats, chickens, turkeys, etc.), and laboratory animals (e.g., mouse, rabbit, rat, guinea pig, fruit fly, etc.).

As used herein, the phrase “treating or inhibiting a microbial infection” means to inhibit the replication of the particular microorganism causing the infection, to inhibit transmission of the microorganism, or to prevent the microorganism from establishing itself in its host, and to ameliorate or alleviate the symptoms of the disease caused by the infection. The treatment is considered therapeutic if there is a reduction in microorganism load, microorganism replication, microorganism counts or cell numbers, decrease in mortality, decrease in symptoms of the infection, such as a fever, and/or morbidity of a subject.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia, For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts, In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or subrnultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The terms “antimicrobial activity,” “microbicidal,” and “microbistatic” refer to the ability of a disclosed compound to modify a function or metabolic process of a target microorganism, for example so as to at least partially affect replication, vegetative growth, toxin production, survival, viability in a quiescent state, or other attribute. Bacteria to be inhibited or killed using the compositions and method described herein can include gram-negative and gram-positive bacteria, in addition to organisms classified in orders of the class Mollicutes and the like, such as species of the Mycoplasma and Acholeplasma genera. Examples of gram-positive bacteria include, but are not limited to, Staphylococcus aureus, Staphylococcus epidermis, Streptococcus agalactiae, Group A streptococcus, Streptococcus pyogenes, Enterococcus faecalis. Group B gram-positive streptococcus, Corynebacterium xerosis, and Listeria monocytogenes. Specific examples of gram-negative bacteria include, but are not limited to, Escherichia coli, Acinetobacter baumannii, Pseudomonas aeruginosa, Klebsiella pneumoniae, Salmonella, Hemophilus influenza, Neisseria gonorrhoeae, Chlamydia trachomatis, Vibrio cholerae, Vibrio parahemolyticus and Helicobacter pylori, Examples of fungi can include yeasts, such as Candida albicans. In some instances, the microbe can appear as either gram-positive or gram-negative, e.g., Mycobacterium tuberculosis, due to a cell surface comprising molecules that make the cells impervious to Gram staining, e.g., the presence of mycotic acid. Accordingly, such organisms, e.g., Mycobacterium tuberculosis, can appear either Gram-negative or Gram-positive.

As used herein, the terms “multidrug-resistant tuberculosis,” “multidrug-resistant TB,” and “MDR TB,” which can be used interchangeably, refer to a form of tuberculosis that is resistant to two or more of the primary drugs (isoniazid and rifampin) used for the treatment of tuberculosis. These terms refer not only to this particular type of the tuberculosis disease, but also to the Mycobacterium tuberculosis that are associated with the disease.

As used herein, the terms “extensively drug-resistant tuberculosis,” “extensively drug-resistant TB,” and “XDR TB.” which can be used interchangeably, refer to a form of tuberculosis that is resistant to at least isoniazid and rifampin among the first-line anti-TB drugs and is resistant to any fluoroquinolone and at least one of the three second-line injectable drugs. These terms refer not only to this particular type of the tuberculosis disease, but also to the Mycobacterium tuberculosis that are associated with the disease.

As used herein, “dosage form” means a pharmacologically active material in a medium, carrier, vehicle, or device suitable for administration to a subject. A suitable dosage form can comprise a compound according to Formula I, a product of a disclosed method of making, or a salt, solvate, or polymorph thereof, in combination with a pharmaceutically acceptable excipient, such as a preservative, buffer, saline, or phosphate buffered saline, Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryollyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound according to Formula I, a product of a disclosed method of making, or a salt, solvate, or polymorph thereof, suspended in sterile saline solution for injection together with a preservative.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, 7-7 interactions, cation-7 interactions, anion-7 interactions, polar 7-interactions, and hydrophobic effects.

The term “biological sample” as used herein refers to a cell or population of cells or a quantity of tissue or fluid from a subject or source, such as an environmental source or a food source, for example. In some embodiments the sample is isolated from or removed from a subject, but, in some embodiments, the term “biological sample” can also refer to cells or tissue analyzed in vivo, i.e. without removal from the subject. Often, a “biological sample” will contain cells from the animal, but the term can also refer to non-cellular biological material. The term biological sample encompasses cellular, tissue or fluid extracts, including, but not limited to, skin, plasma, serum, spinal fluid, lymph fluid, synovial fluid, urine, tears, blood cells, organs, tumors, and also to samples of in vitro cell culture constituents (including, but not limited to, conditioned medium resulting from the growth of cells (including prokaryotic and eukaryotic cells) in cell culture medium, recombinant cells, and cell components). Samples can comprise cellular or tissue explants obtained from an individual or organism during a medical procedure or intervention, such as a surgical procedure or biopsy. Samples from environmental sources are also included among “samples” to which the compositions and methods described herein can be applied.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “therapeutic” can refer to treating, healing, and/or ameliorating a disease, disorder. condition, or side effect, or to decreasing in the rate of advancement of a disease, disorder, condition, or side effect.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action, A therapeutic agent can be a primary active agent. or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs. vaccines. and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics. antiasthmatic agents, anticonvulsants, antihistamines, antinauseants, antineoplastics, antipruritics, antipyretics; antispasmodics_{;} cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example. proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs. which become biologically active or more active after they have been placed in a predetermined physiological environment.

The term ‘pharmaceutically acceptable’ describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, the term “pharmaceutically acceptable carrier” refers to aqueous or nonaqueous solutions, dispersions, suspensions or emulsions, as well as powders for reconstitution into injectable solutions or dispersions just prior to use. Preferably, a pharmaceutically acceptable carrier will be sterile or sterilizable, e.g., where the pharmaceutical composition is intended for injection. The pharmaceutically acceptable carrier is advantageously selected so as not to significantly decrease or neutralize the active ingredient. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0,01 to 10 micrometers.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority. EZ specification, and the like. One of skill in the art can readily ascertain the structure of a compound if given a name, either by systemic reduction of the compound structure using naming conventions, or by commercially available software, such as CHEMBIODRAW™ (Cambridgesoft Corporation, U.S.A.). Compounds were generally named herein using CHEMBIODRAW™ (v. 14.0.0.117).

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A², ” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms, Aliphatic groups include, but are not limited to. linear or branched. alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl. decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfa-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group.

For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon.

The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein, That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂—(methylene), —CH₂CH₂—, —CH₂C(CH₃)₂CH₂, and CH₂CH₂CH₂are non-limiting examples of alkanediyl groups.

The term “aromatic group” as used herein refers to a ring structure having cyclic clouds of delocalized π electrons above and below the plane of the molecule, where the π clouds contain (4n+2)π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH_{2.}

The term “alkylamino” as used herein is represented by the formula —NH(-alkyl) or —N(-alkyl)₂, where alkyl is a described herein. Representative examples include, but are not limited to, methylamino group, ethylamino group, propylamino group, isopropylarnino group, butylamino group, isobutylarnino group, (sec-butyl)amino group, (tert-butyl)amino group, pentylamino group, isopentylamino group, (tert-pentyl)amino group, hexylamino group, dimethylaniino group, diethylamino group, dipropylamino group, diisopropylamino group, dibutylamino group, diisobutylamino group, di(sec-butyl)amino group, di(tert-butyl)arnino group, dipentylamino group, diisopentylarnino group, di(tert-pentyl)amino group, dihexylamino group, N-ethyl-N-methylamino group, N-methyl-N-propylamino group, N-ethyl-N-propylamino groupand the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thins, thioxos and the like.

The term “ester,” as used herein, include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

The terms “sulfinyl” and “thienyl,” as used herein can be used interchangeably and refer to a group comprising —S(O)—.

The terms “sulfonyl” and “sulfuryl,” as used herein can be used interchangeably and refer to a group comprising comprising —S(O)₂.

The term “sulfo,” as used herein, refers to a group comprising —S(O)₃—.

The term “sulfonate,” as used herein, refers to a group having a structure represented by a formula —SO₂OR, where R is hydrogen, C1-6 alkyl, C2-6 alkenyl, C2-6 alkynyl, C3-10 cycloalkyl, heterocyclyl, aryl, or heteroaryl.

The term “sulfonyl,” as used herein, refers to a group having a structure represented by a formula —SO₂R, where R is hydrogen, C1-6 alkyl, C2-6 alkenyl, C2-6 alkynyl, C3-10 cycloalkyl, heterocyclyl, aryl, or heteroaryl,

The term “phosphate,” as used herein, refers to a pentavalent phosphorous group having the formula —OP(═O)(OR′)(OR″), where each R′ and R″ is selected, independently, from hydrogen, C1-6 alkyl, C2-6 alkenyl, C2-6 alkynyl, C3-10 cycloalkyl, heterocyclyl, aryl, or heteroaryl.

The terms “hydroxyl” and “hydroxyl,” as used herein can be used interchangeably and refer to a moiety having a structure represented by the formula —OH.

The terms “nitrile” and “cyano,” as used herein can be used interchangeably and refer to a moiety having a structure represented by the formula —CN.

The term “haloalkoxy,” as used herein, refers to a C1-C6 haloalkyl group attached to the parent molecular moiety through an oxygen atom, e.g., —OCH₂CHF₃.

The terms “halo.” “halogen.” or “halide,” as used herein can be used interchangeably and refer to F, Cl. Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanate, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e. attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the disclosure may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15. carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

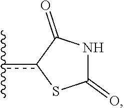

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present disclosure unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical, In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the disclosure includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereorners and optical isomers, Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*), When bonds to the chiral carbon are depicted as straight lines in the disclosed formulas, it is understood that both the (R) and (3) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, phosphorous, fluorine and chlorine, such as ² H, ³H, ¹³C, ¹⁴C, ¹⁵N, ¹⁸O, ¹⁷O, ³⁵S, ¹⁸F and ³⁸Cl, respectively. Compounds further comprise prodrugs thereof, and pharmaceutically acceptable salts of said compounds or of said prodrugs which contain the aforementioned isotopes and/or other isotopes of other atoms are within the scope of this disclosure, Certain isotopically-labeled compounds of the present disclosure, for example those into which radioactive isotopes such as ³H and ¹⁴C are incorporated, are useful in drug and/or substrate tissue distribution assays, Tritiated, i.e., ³H, and carbon-14, i.e., ¹⁴C, isotopes are particularly preferred for their ease of preparation and detectability. Further, substitution with heavier isotopes such as deuterium, i.e., ² H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

The compounds described in the disclosure can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the disclosure to form solvates and hydrates. Unless stated to the contrary, the disclosure includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an o-hydrogen can exist in an equilibrium of the keto form and the enol form.

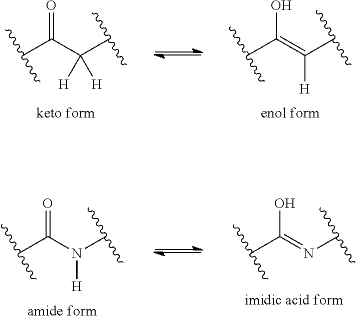 [SMILES: *CC(*)=O ; */C=C(/*)O ; *NC(*)=O ; */N=C(/*)O]

Likewise, amides with an N-hydrogen can exist in an equilibrium of the amide form and the iridic acid form. As another example, pyrazoles can exist in two tautomeric forms, N¹-unsubstituted, 3-A³ and N¹-unsubstituted, 5-A³ as shown below.

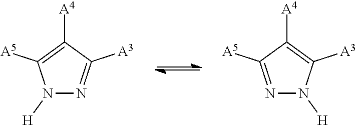

Unless stated to the contrary, the disclosure includes all such possible tautomers.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

In some aspects, a structure of a compound can be represented by a formula:

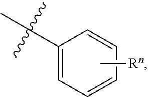

which is understood to be equivalent to a formula:

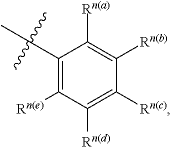

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

In some aspects, a structure of a compound can be represented by a formula:

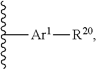

which is understood to be equivalent to a group, Ar¹, that at least comprises a substituent group, R²⁰, in any position on the Ar¹ group in which such a substituent group is permitted in order to satisfy valency. That is, unless stated otherwise, the Ar¹ group comprises the substituent group, R²⁰, as well as any other substituent groups that are defined for the Ar¹ group herein. In some instances, it will be specified that the foregoing structure corresponds to an Ar1 that is monosubstituted with a substituent group, R²⁰.

In some aspects, similarly, a structure of a compound can be represented by a formula:

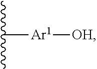

which is understood to be equivalent to a group, Ar¹, that at least comprises a substituent group, —OH, in any position on the Ar¹ group in which such a substituent group is permitted in order to satisfy valency. That is, unless stated otherwise, the Ar¹ group comprises the substituent group, —OH, as well as any other substituent groups that are defined for the Ar¹ group herein. In some instances, it will be specified that the foregoing structure corresponds to an AO that is monosubstituted with a substituent group, —OH.

A weight percent (wt. %) of a component, unless specifically stated to the contrary, is based on the total weight of the formulation or composition in which the component is included.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Ws.), Acros Organics (Morris Plains, N.J,), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In accordance with the purpose(s) of the disclosure, as embodied and broadly described herein, the disclosure, in one aspect, relates to substituted spectinomycin analogs, including substituted aminomethyl spectinomycin analogs and substituted spectinamide analogs, with increased tolerability and safety, including improved tolerability to parenteral administration. More specifically, in one aspect, the present disclosure relates to compounds that are aryl substituted aminomethyl spectinomycin analogs or aryl substituted spectinoarnide analogs comprising a prodrug moiety, e.g., a phospho- or sulfa-ester derivative, that are uesful for treating bacterial infections.

It is understood that reference to a disclosed compound is inclusive of the disclosed compound, as well as pharmaceutically acceptable salt, hydrate, solvate, or polymorph forms thereof.

It is contemplated that each disclosed derivative can be optionally further substituted. It is also contemplated that any one or more derivative can be optionally omitted from the disclosure. It is understood that a disclosed compound can be provided by the disclosed methods. It is also understood that the disclosed compounds can be employed in the disclosed methods of using.

In one aspect, the disclosure relates to a compound having a structure represented by a formula:

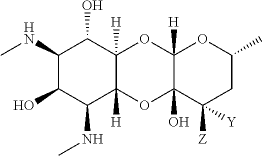

wherein Y is hydrogen or hydroxyl; wherein Z is —CH₂—NH—(C1-C3 alkanediyl)-Ar¹ or —NH—(C═O)—(C1-C3 alkanediyl)Ar¹; wherein Ar¹ is aryl or heteroaryl substituted with: (a) a R²⁰ group, wherein R²⁰ is selected from —(C1-C3)-alkanediyl—OP(O)(OR²¹) (OR²²), —OP(O)(OR²¹)(OR²²), —(C1-C3)-alkanediyl-OSO₂OR²¹, —OSO₂OR²¹, —(C1-C3)-alkanediyl-OSO_{NR}²¹R²², —OSO₂NR²¹R²², wherein each of R²¹ and R²² is independently selected from hydrogen and C1-C3 alkyl; and (b) 0 to 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

In one aspect, the disclosure relates to a compound having a structure represented by a formula:

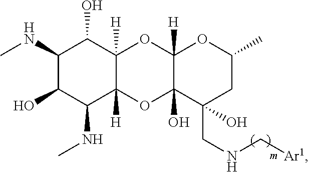

wherein m is an integer selected from 0, 1, 2, and 3; wherein Art is aryl or heteroaryl substituted with: (a) a R²⁰ group, wherein R²⁰ is selected from (C1-C3)-alkanediyl-OP(O)(OR²¹) (OR²²), —OP(O)(OR²¹)(OR²²), (C1-C3)-alkanediyl-OSO₂OR²¹, OSO₂OR²¹, —(C1-C3)-alkanediyl-OSO₂R²¹, —OSO₂R²¹, —(C1 -C3)-alkanediyl-OSO₂NR²¹R²², —OSO₂NR^{21 22}, wherein each of R²¹ and R²² is independently selected from hydrogen and C1-C3 alkyl; and (b) 0 to 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

In one aspect, the disclosure relates to a compound having a structure represented by a formula:

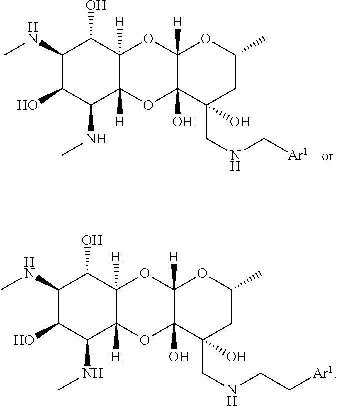

In one aspect, the disclosure relates to a compound having a structure represented by a formula:

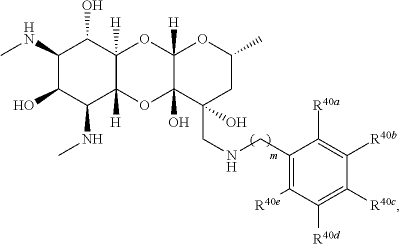

wherein m is an integer selected from 0, 1, 2, and 3; wherein 1 of R^{40a}, R^{40b}, R^{40c}, R^{40d}, and R^{40e} is R²⁰; wherein 2, 3, or 4 of R^{40a}, R^{40b}, R^{40c}, R^{40d}, and R^{40e} are independently hydrogen; and, wherein 0, 1, or 2 of R^{40a}, R^{40b}, R^{40c}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

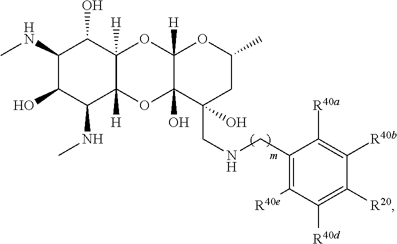

wherein m is an integer selected from 0, 1, 2, and 3; wherein R²⁰ is selected from —(C1-C3)-alkanediyl-OP(O)(OR²¹) (OR²²), —OP(O)(OR²¹)(OR²²), —(C1-C3)-alkanediyl-OSO₂OR²¹, —OSO₂OR²¹, —(C1-C3)-alkanediyl-OSO₂R²¹, —OSO₂R²¹, —(C1-C3)-alkanediyl-OSO₂NR²¹ R²², —OSO₂NR²¹R²², wherein each of R²¹ and R²² is independently selected from hydrogen and C1-C3 alkyl; wherein 0, 1, or 2 of R^{40a}, R^{40b}, R^{40c}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

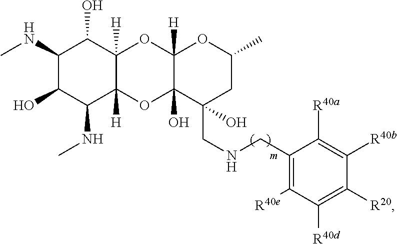

wherein m is an integer selected from 0, 1, 2, and 3; wherein R²⁰ is selected from —(C1-C3)-alkanediyl-OP(O)(OR²¹) (OR²²), —OP(O)(OR²¹)(OR²²), —(C1-C3)-alkanediyl-OSO₂OR²¹, —OSO₂OR²¹, —(C1-C3)-alkanediyl-OSO₂R²¹, —OSO₂R²¹, —(C1-C3)-alkanediyl-OSO₂NR²¹ R²², —SO₂NR²¹R²², wherein each of R²¹ and R²² is independently selected from hydrogen and C1-C3 alkyl; wherein each of R^{40a}, R^{40b}, R^{40d}, and R^{40e} is hydrogen.

In one aspect, the disclosure relates to a compound having a structure represented by a formula:

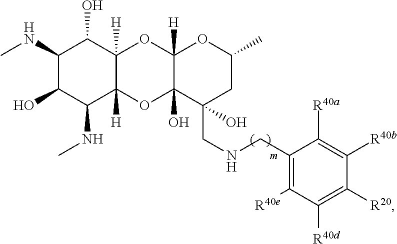

wherein m is an integer selected from 0, 1, 2, and 3; wherein R²⁰ is selected from —(C1-C3)-alkanediyl-OP(O)(OR²¹) (OR²²), OP(O)(OR²¹)(OR²²), (C1-C3)-alkanediyl-OSO₂OR²¹, OSO₂OR²¹, (C1 -C3)-alkanediyl-OSO₂R²¹, —OSO₂R²¹, —(C1-C3)-alkanediyl-OSO₂NR²¹R²², —SO₂NR²¹R²², wherein each of R²¹ and R²² is independently selected from hydrogen and C1-C3 alkyl; wherein R^{40a} is hydrogen or halo; and wherein each of R^{40b}, R^{40d}, and R^{40e} is hydrogen.

In one aspect, the disclosure relates to a compound having a structure represented by a formula:

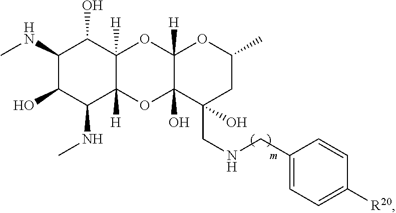

wherein m is an integer selected from 0, 1, 2, and 3; wherein R²⁰ is selected from (C1-C3)-alkanediyl-OP(O)(OR²¹) (OR²²), OP(O)(OR²¹)(OR²²), (C1-C3)-alkanediyl-OSO₂OR²¹, —OSO₂OR²¹, (C1 -C3)-alkanediyl-OSO₂R²¹, OSO₂R²¹, (C1-C3)-alkanediyl-OSO₂NR²¹ R²², —OSO₂NR²¹R²², wherein each of R²¹ and R²² is independently selected from hydrogen and C1-C3 alkyl.

In one aspect, the disclosure relates to a compound having a structure represented by a formula:

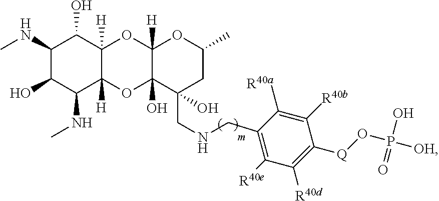

wherein m is an integer selected from 0, 1, 2, and 3; wherein Q is —(C0-C3)-alkanediyl; and wherein 0, 1, or 2 of R^{40a}, R^{40b}, R^{40d}, and R^{40e} are independently selected from halo, cyano, hydroxyl, —NH₂, C1-C3 alkyl. C1-C3 haloalkyl, C1-C3 alkoxy, and C1-C3 haloalkoxy.

In one aspect, the disclosure relates to a compound having a structure represented by a formula:

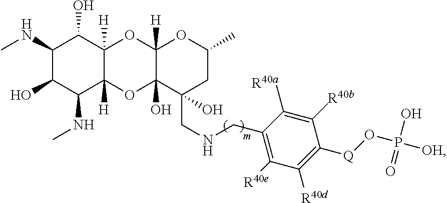

wherein m is an integer selected from 0, 1, 2, and 3; wherein Q is —(C0-C3)-alkanediyl; and wherein each of R^{40a}, R^{40b}, R^{40d}, and R^{40e} is hydrogen.

In one aspect, the disclosure relates to a compound having a structure represented by a formula:

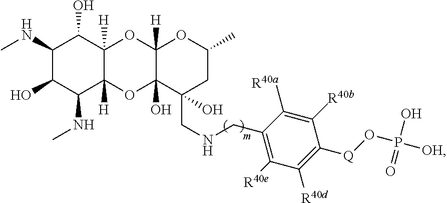

wherein m is an integer selected from 0, 1, 2, and 3; wherein Q is —(C0-C3)-alkanediyl; and wherein R^{40a} is hydrogen or halo; and wherein each of R^{40b}, R^{40d}, and R^{40e} is hydrogen.

In one aspect, the disclosure relates to a compound having a structure represented by a formula:

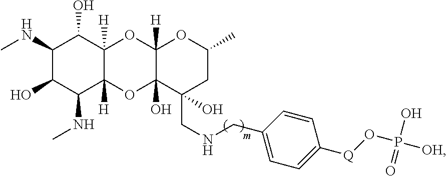

wherein m is an integer selected from 0, 1, 2, and 3; and wherein Q is —(C0-C3)-alkanediyl,

In one aspect, the disclosure relates to a compound having a structure represented by a formula:

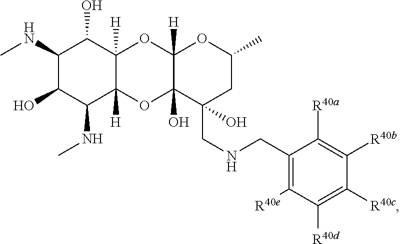

wherein 1 of R^{40a}, R^{40b}, R^{40c}, R^{40d}, and R^{40e} is R²⁰; wherein 2, 3, or 4 of R^{40a}, R^{40b}, R^{40c}, R^{40d}, and R^{40e} are independently hydrogen; and, wherein 0, 1, or 2 of R^{40a}, R^{40b}, R^{40c}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

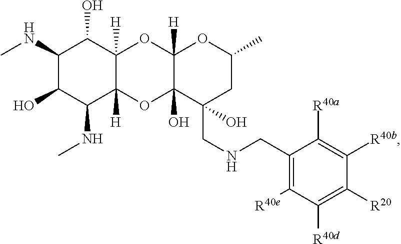

wherein R²⁰ is selected from —(C1-C3)-alkanediyl-OP(O)(OR²¹) (OR²²), —OP(O)(OR²¹)(OR²²), (C1-C3)-alkanediyl-OSO₂OR²¹, —OSO₂OR²¹, (C1-C3)-alkanediyl-OSO₂R²¹, —OSO₂R²¹, —(C1-C3)-alkanediyl-OSO₂NR²¹R²², —OSO₂N R²¹ R²², wherein each of R²¹ and R²² is independently selected from hydrogen and C1-C3 alkyl; wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

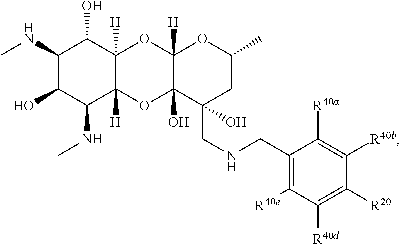

wherein R²⁰ is selected from (C1-C3)-alkanedlyl-OP(O)(OR²¹) (OR²²), —OP(O)(OR²¹)(OR²²), —(C1-C3)-alkanediyl-OSO₂OR²¹, —OSO₂OR²¹, (C1-C3)-alkanediyl-OSO₂R²¹, CS0₂R²¹, (C1-C3)-alkanediyl-OSO₂NR²¹R²², —OSO₂NR²¹R²², wherein each of R²¹ and R²² is independently selected from hydrogen and C1-C3 alkyl; wherein each of R^{40a}, R^{40b}, R^{40d} and R^{4e} is hydrogen.

In one aspect, the disclosure relates to a compound having a structure represented by a formula:

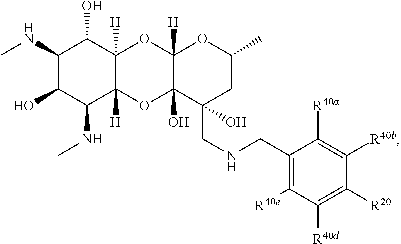

wherein R²⁰ is selected from (C1-C3)-alkanediyl-OP(O)(OR²¹) (R²²), —OP(O)(OR²¹)(OR²²), (C1-C3)-alkanediyl-OSO₂OR²¹, —OSO₂OR²¹, —(C1-C3)-alkanediyl-OSO₂R²¹, —OSO₂R²¹, —(C1-C3)-alkanediyl-OSO₂NR²¹R²², —OSO₂NR²¹R₂₂, wherein each of R²¹ and R²² is independently selected from hydrogen and C1-C3 alkyl; wherein R^{40a} is hydrogen or halo; and wherein each of R^{4Ob}, R^{40d}, and R^{40e} is hydrogen.

In one aspect, the disclosure relates to a compound having a structure represented by a formula;

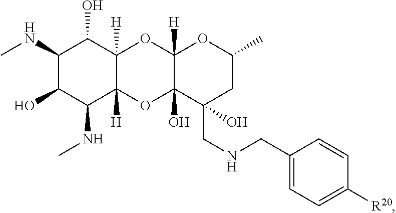

wherein R²⁰ is selected from (C1-C3)-alkanediyl-OP(O)(OR²¹) (OR²²), —OP(O)(OR²¹)(OR²²), —(C1-C3)-alkanediyl-OSO₂OR^{21, —OSO}₂OR²¹, (C1-C3)-alkanediyl-OSO₂R²¹, —OSO₂R²¹, —(C1-C3)-alkanediyl-OSO₂NR²¹R²², —OSO₂NR²¹R²², wherein each of R²¹ and R²² is independently selected from hydrogen and C1-C3 alkyl.

In one aspect, the disclosure relates to a compound having a structure represented by a formula:

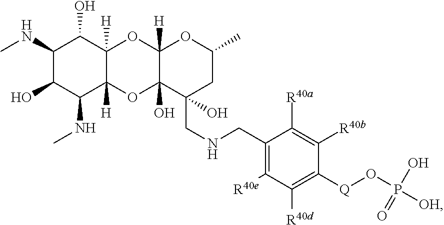

wherein Q is —(C0-C3)-alkanediyl; and wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound haying a structure represented by a formula:

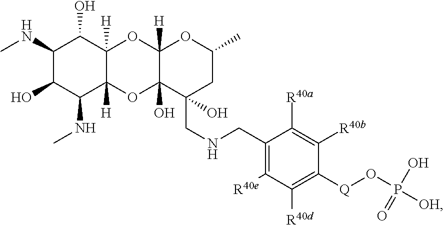

wherein Q is —(C0-C3)-alkanediyl; and wherein each of R^{40a}, R^{40b}, R^{40d}, and R^{40e} is hydrogen.

In one aspect, the disclosure relates to a compound haying a structure represented by a formula:

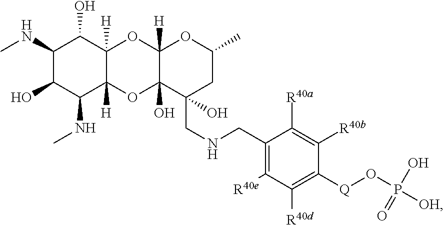

wherein Q is —(C0-C3)-alkanediyl; and wherein FR⁴⁰a is hydrogen or halo; and wherein each of R^{40b}, R^{4d}, and R^{40e} is hydrogen.

In one aspect, the disclosure relates to a compound haying a structure represented by a formula:

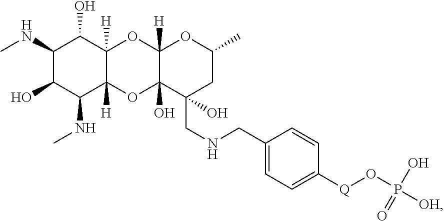

wherein Q is —(C0-C3)-alkanediyl.

In one aspect, the disclosure relates to a compound haying a structure represented by a formula:

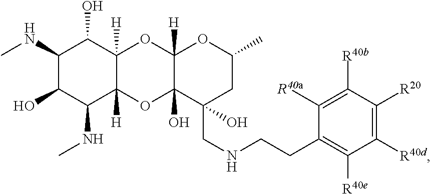

wherein 1 of R^{40a}, R^{40b}, R^{40c}, R^{40d} and R^{40e} is R²⁰; wherein 2, 3, or 4 of R^{40a}, R^{40 b}, R^{40c}, R^{40d}, and R^{40e} are independently hydrogen; and, wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula;

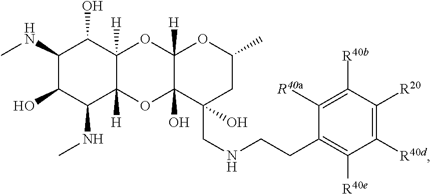

wherein R²⁰ is selected from —(C1-C3)-alkanediyl-OP(O)(OR²¹) (OR²²), OP(O)(OR²¹)(OR²²), (C1-C3)-alkanediyl-OSO₂OR²¹, —OSO₂OR²¹, (C1-C3)-alkanediyl-OSO₂R²¹, —OSO₂R²¹, —(C1-C3)-alkanediyl-OSO₂NR²¹R²², —OSO₂NR²¹ R²² , wherein each of R²¹ and R²² is independently selected from hydrogen and C1-C3 alkyl; wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

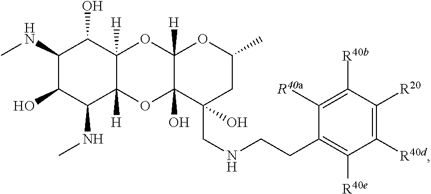

wherein R²⁰ is selected from (C1-C3)-alkanediyl-OP(O)(OR²¹) (OR²²), OP(O)(OR²¹)(OR²²), (C1-C3)-alkanedlyl-OSO₂OR²¹, —OSO₂OR²¹, —(C1-C3)-alkanediyl-OSO₂R²¹, —OSO₂R²¹, —(C1-C3)-alkanediyl-OSO₂NR²¹R²², —OSO₂NR²¹R²², wherein each of R²¹ and R²² is independently selected from hydrogen and C1-C3 alkyl; wherein each of R^{40a}, R^{40b}, R^{40d}, and R^{40e} is hydrogen.

In one aspect, the disclosure relates compound having a structure represented by a formula:

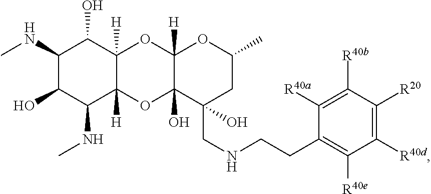

wherein R²⁰ is selected from (C1-C3)-alkanedlyl-OP(O)(OR²¹) R²²), —OP(O)(OR²¹)(OR²²), —(C1-C3)-alkanediyl-OSO₂OR²¹, —OSO₂OR²¹, (C1-C3)-alkanediyl-OSO₂R²¹, —OSO₂R²¹, —(C1-C3)-alkanediyl-OSO₂NR²¹R²², OSO₂NR²¹R²², wherein each of R²¹ and R²² is independently selected from hydrogen and C1-C3 alkyl; wherein R^{40a} is hydrogen or halo; and wherein each of R^{40b}, R^{40d}, and R^{40e} is hydrogen.

In one aspect, the disclosure relates to a compound having a structure represented by a formula:

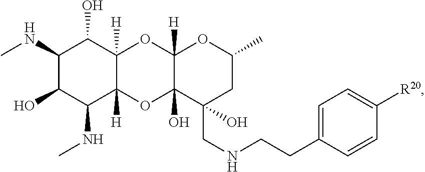

wherein R²⁰ is selected from (C1-C3)-alkanediyl-OP(O)(OR²¹) (OR²²), CP(O)(OR²¹)(OR²²), —(C1-C3)-alkanediyl-OSO₂OR²¹, —OSO₂OR²¹, (C1-C3)-alkanediyl-OSO₂R²¹, —OSO₂R²¹, —(C1-C3)-alkanediyl-OSO₂NR²¹R², OSO₂NR²¹R²², wherein each of R²¹ and R²² is independently selected from hydrogen and C1-C3 alkyl.

In one aspect, the disclosure relates to a compound having a structure represented by a formula:

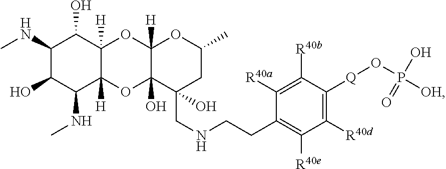

wherein Q is —(C0-C3)-alkanediyl; and wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and 1-C3 haloalkoxy.

In one aspect, the disclosure relates to a compound having a structure represented by a formula:

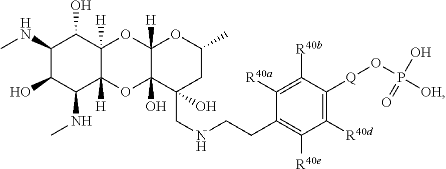

wherein Q is —(C0-C3)-alkanediyl; and wherein each of R^{40a}, R^{40b}, R^{40d}, and R^{40e} is hydrogen.

In one aspect, the disclosure relates to a compound having a structure represented by a formula;

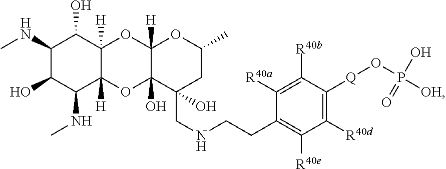

wherein Q is —(C0-C3)-alkanediyl; and wherein R^{40a} is hydrogen or halo; and wherein each of R^{40b}, R^{40d}, and R^{40e} is hydrogen.

In one aspect, the disclosure relates to a compound having a structure represented by a formula:

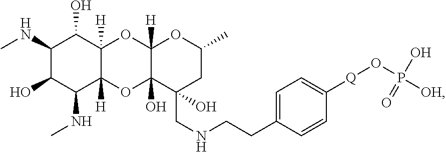

wherein Q is —(C0-C3)-alkanediyl.

In one aspect, the disclosure relates to a compound having a structure represented by a formula:

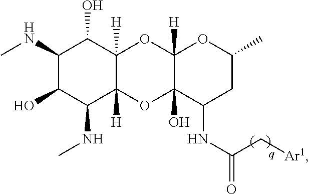

wherein g is an integer selected from 0, 1, 2, and 3; wherein Ar¹ is aryl or heteroaryl substituted with: (a) a R²⁰ group, wherein R²⁰ is selected from (C1-C3)-alkanedlyl-OP(O)(OR²¹) (OR²²), —OP(O)(OR²¹)(OR²²), (C1-C3)-alkanediyl-OSO₂OR²¹, —OSO₂OR²¹, (C1-C3)-alkanediyl-OSO₂R²¹, —OSO₂R²¹, (C1-C3)-alkanediyl-OSO₂NR²¹R²², —OSO₂NR²¹R²², wherein each of R²¹ and R²² is independently selected from hydrogen and C1-C3 alkyl; and (b) 0 to 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

In one aspect, the disclosure relates to a compound having a structure represented by a formula;

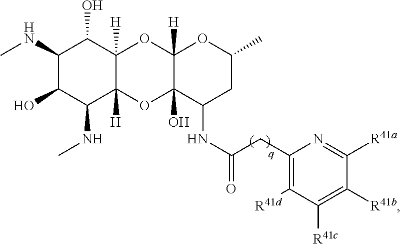

wherein q is an integer selected from 0, 1, 2, and 3; wherein 1 of R^{41a}, R^{41b}, R^{41c}, and R^{41d} is R²⁰; wherein 1, 2, or 3 of R^{41a}, R^{41b}, and R^{41d} are independently hydrogen; and, wherein 0, 1, or 2 of R^{41a}, R^{41b}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

In one aspect, the disclosure relates to a compound having a structure represented by a formula:

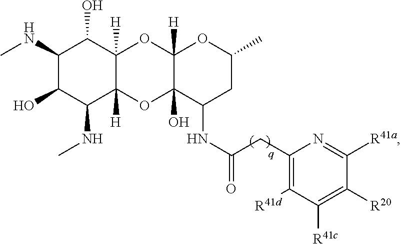

wherein q is an integer selected from 0, 1, 2, and 3; wherein R²⁰ is selected from (C1-C3)-alkanediyl-OP(O)(OR²¹) (OR²²), OP(O)(OR²¹)(OR²²), (C1-C3)-alkanediyl-OSO₂OR²¹, —OSO₂OR²¹, —(C1 -C3)-alkanediyl-OSO₂R²¹, OSO₂R²¹, (C1-C3)-alkanediyl-OSO₂NR²¹R²², —OSO₂NR²¹R²², wherein each of R²¹ and R²² is independently selected from hydrogen and C1-C3 alkyl; and wherein 0, 1, or 2 of R^{41a}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

In various aspects, a disclosed compound is selected from a structure represented by a formula:

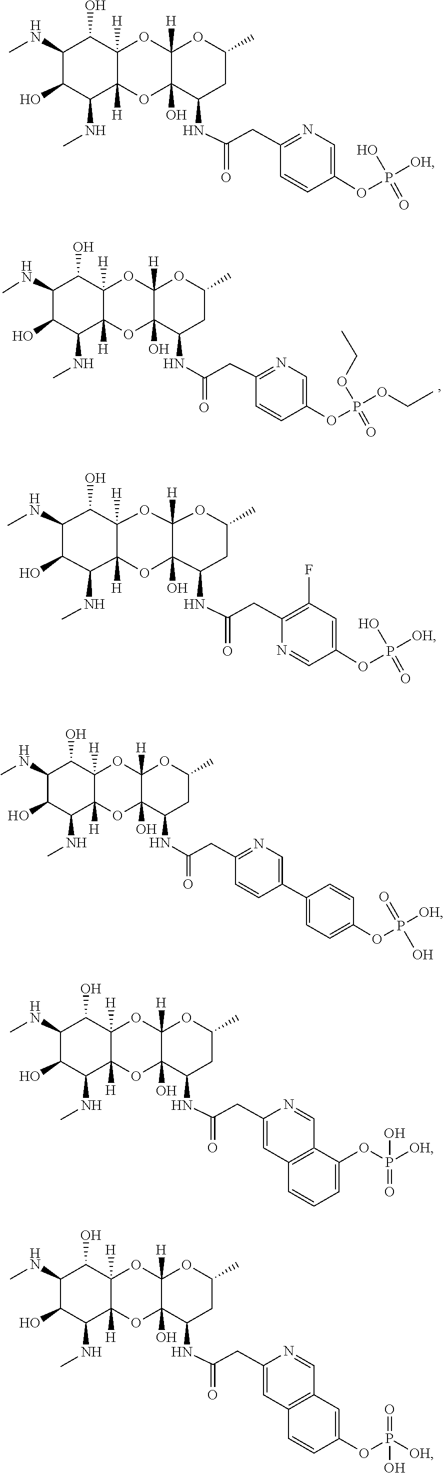

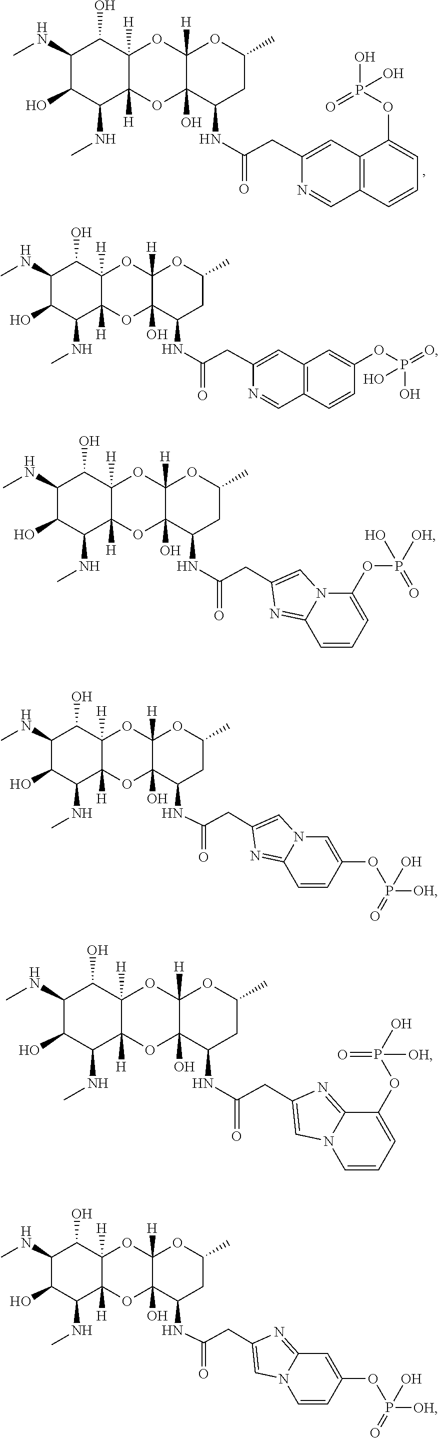

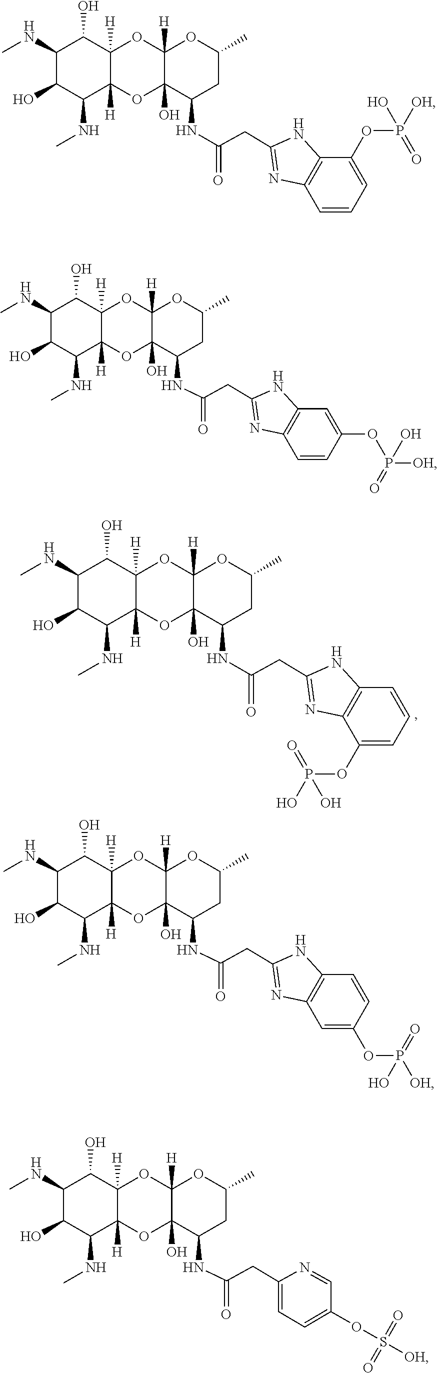

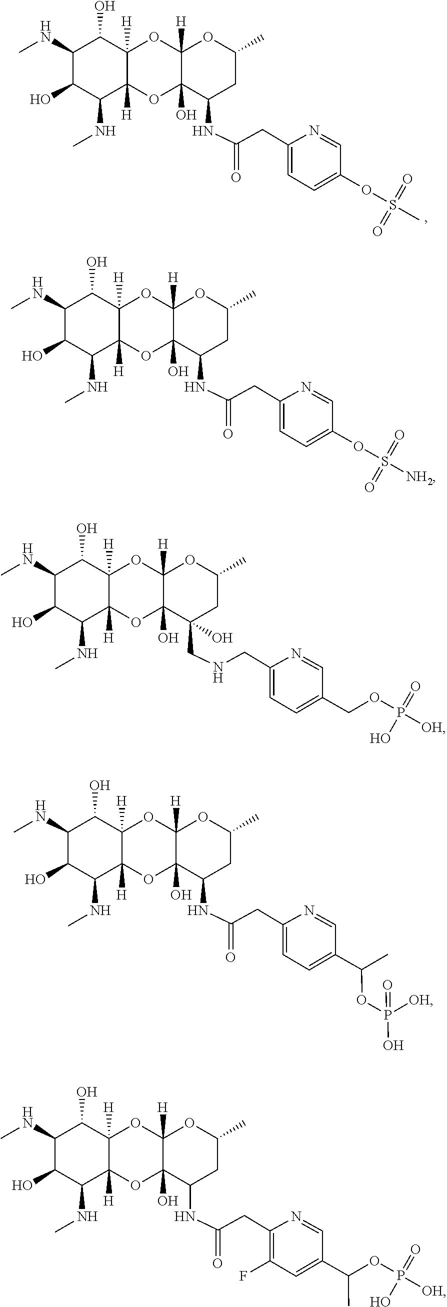

and combinations thereof.

In a further apsect, a disclosed compound is selected from a structure represented by a formula:

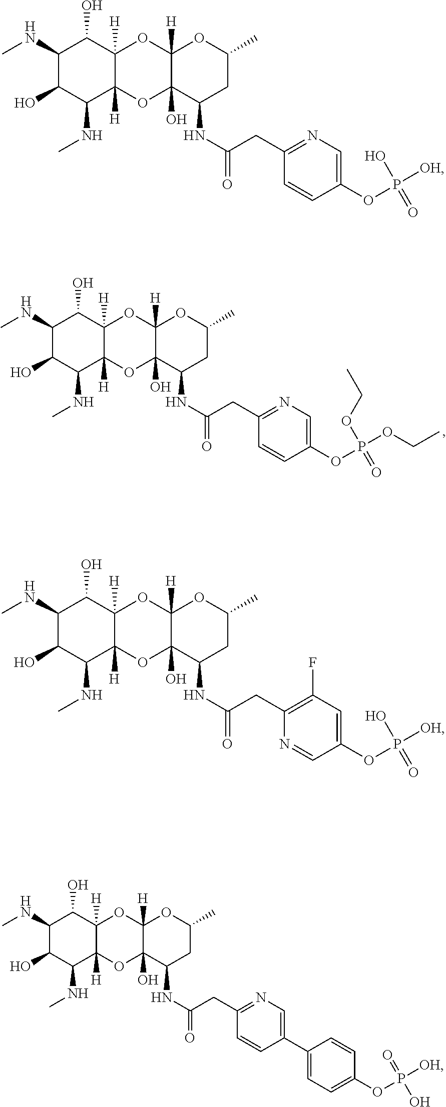

and combinations thereof.

In a further aspect, a disclosed compound has a structure represented by a formula:

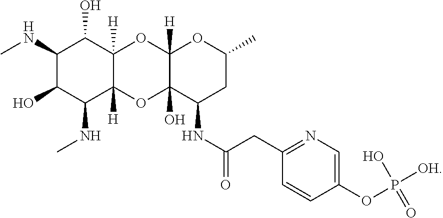 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1ccc(OP(=O)(O)O)cn1]

In various aspects, a disclosed compound is selected from a structure represented by a formula:

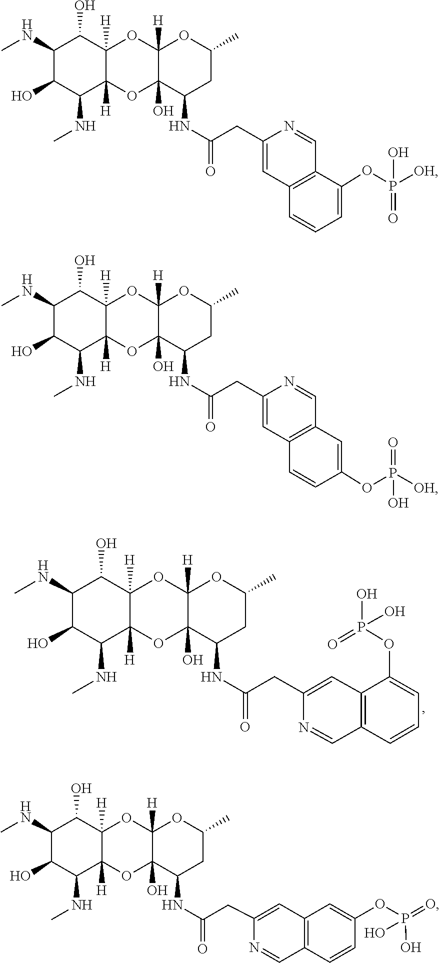 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1cc2cccc(OP(=O)(O)O)c2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2ccc(OP(=O)(O)O)cc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2c(OP(=O)(O)O)cccc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2cc(OP(=O)(O)O)ccc2cn1]

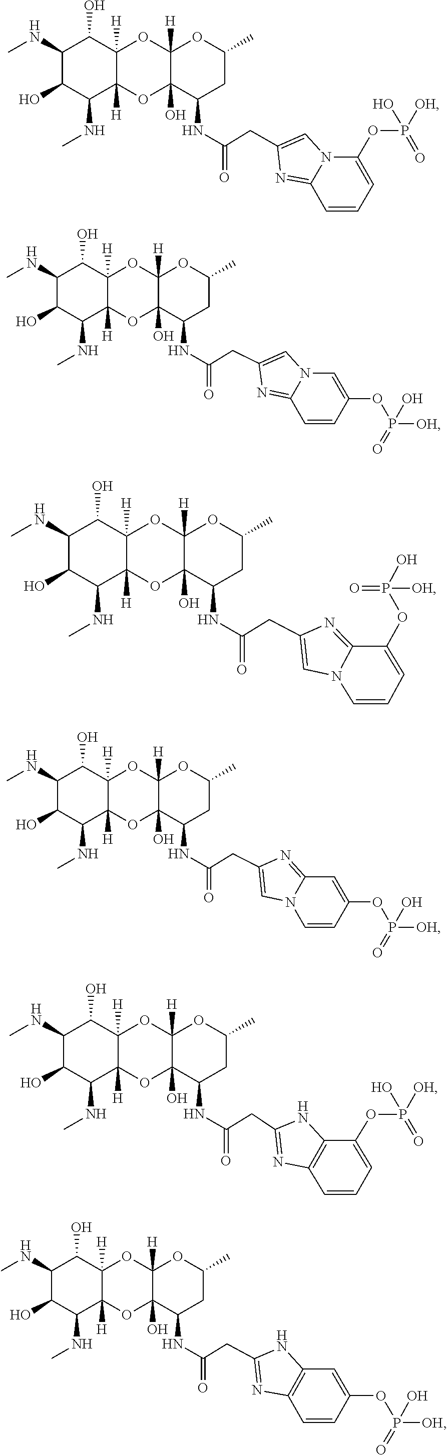

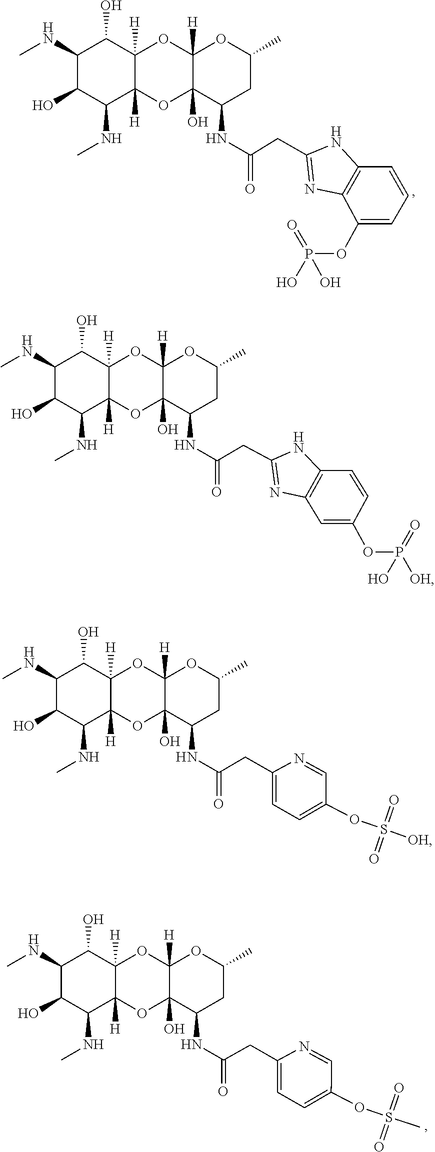

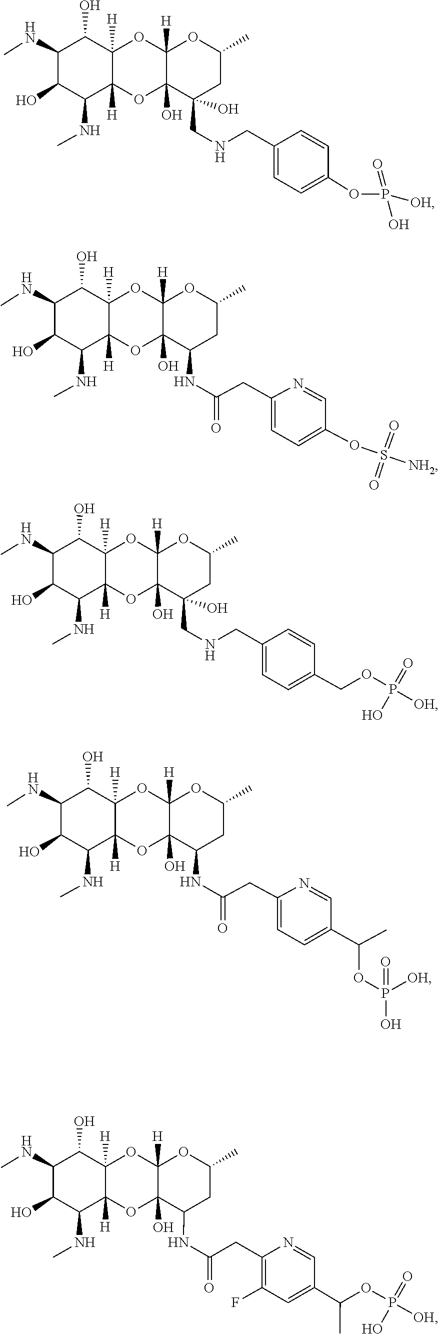

and combinations thereof.

In a further aspect, a disclosed compound is selected from a structure represented by a formula:

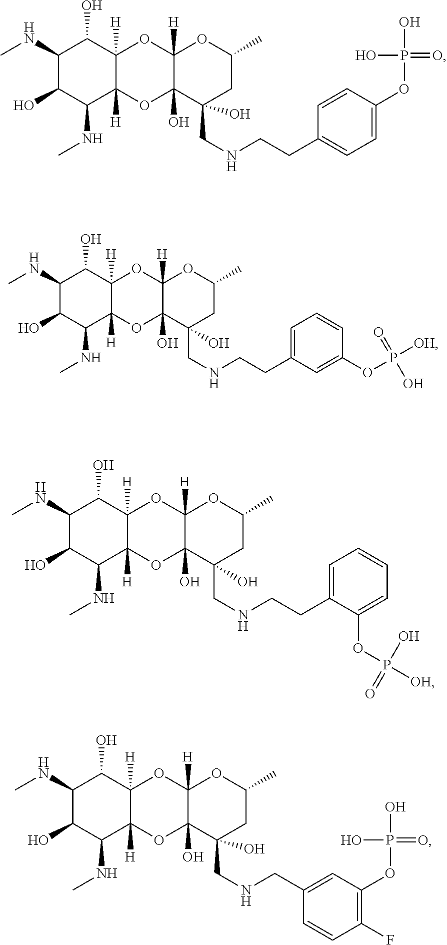 [SMILES: CN[C@@H]1[C@H](O)[C@H](NC)[C@H]2O[C@]3(O)[C@H](O[C@@H]2[C@H]1O)O[C@H](C)C[C@@]3(O)CNCCc1ccc(OP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCCc1cccc(OP(=O)(O)O)c1 ; CN[C@@H]1[C@H](O)[C@H](NC)[C@H]2O[C@]3(O)[C@H](O[C@@H]2[C@H]1O)O[C@H](C)C[C@@]3(O)CNCCc1ccccc1OP(=O)(O)O ; CN[C@@H]1[C@H](O)[C@H](NC)[C@H]2O[C@]3(O)[C@H](O[C@@H]2[C@H]1O)O[C@H](C)C[C@@]3(O)CNCc1ccc(F)c(OP(=O)(O)O)c1]

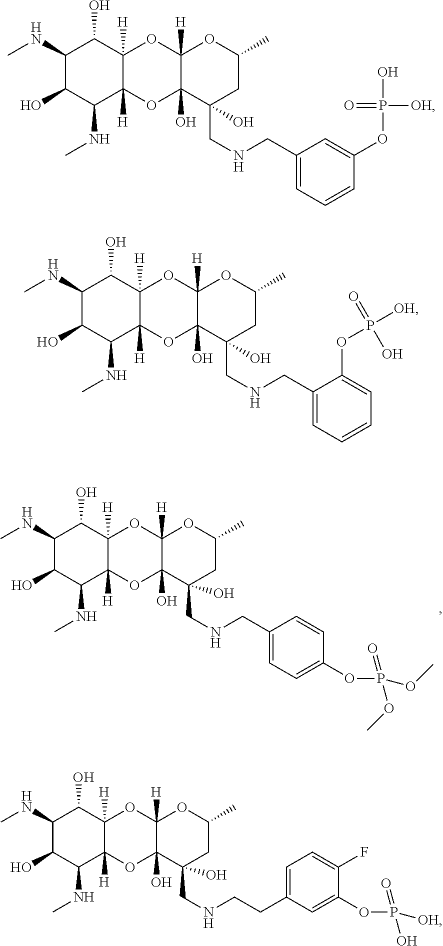

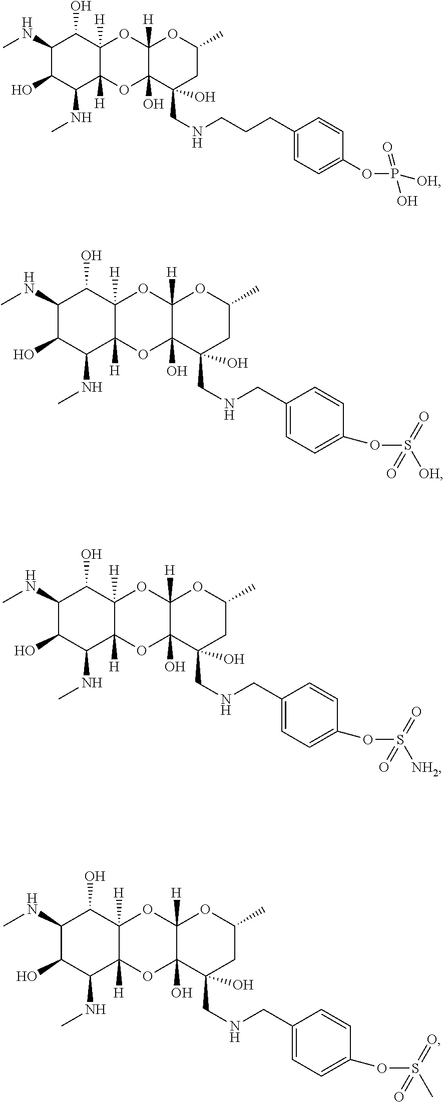 [SMILES: CN[C@@H]1[C@H](O)[C@H](NC)[C@H]2O[C@]3(O)[C@H](O[C@@H]2[C@H]1O)O[C@H](C)C[C@@]3(O)CNCCCc1ccc(OP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(=O)(=O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(N)(=O)=O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(C)(=O)=O)cc1]

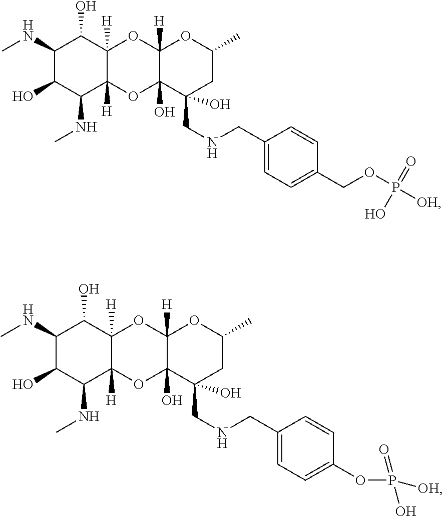 [SMILES: CN[C@@H]1[C@H](O)[C@H](NC)[C@H]2O[C@]3(O)[C@H](O[C@@H]2[C@H]1O)O[C@H](C)C[C@@]3(O)CNCc1ccc(COP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OP(=O)(O)O)cc1]

and combinations thereof.

In a further aspect, a disclosed compound is selected from a structure represented by a formula:

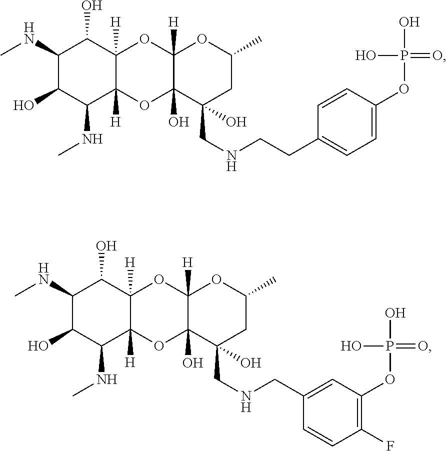 [SMILES: CN[C@@H]1[C@H](O)[C@H](NC)[C@H]2O[C@]3(O)[C@H](O[C@@H]2[C@H]1O)O[C@H](C)C[C@@]3(O)CNCCc1ccc(OP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(F)c(OP(=O)(O)O)c1]

and combinations thereof.

In various aspects, a disclosed compound is selected from a structure represented by a formula:

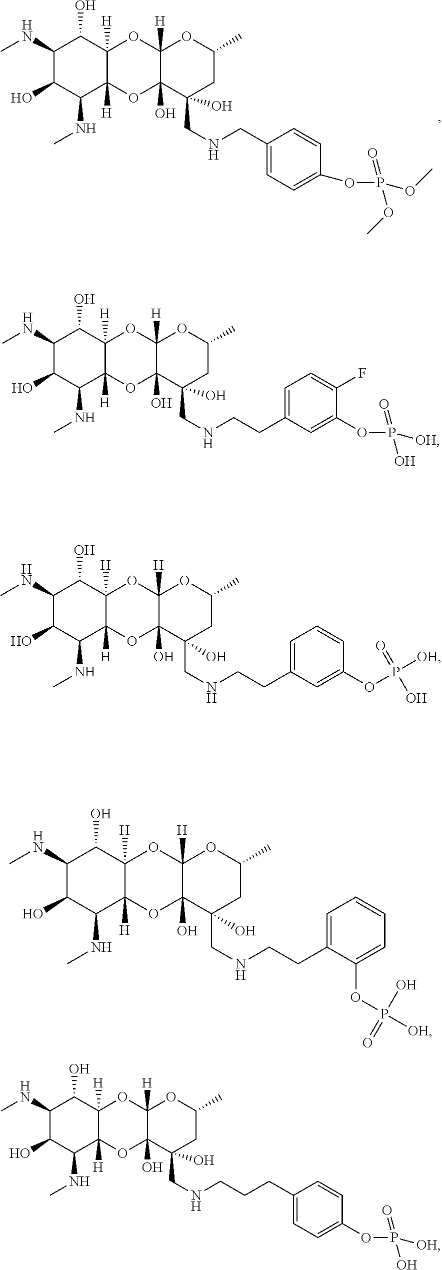

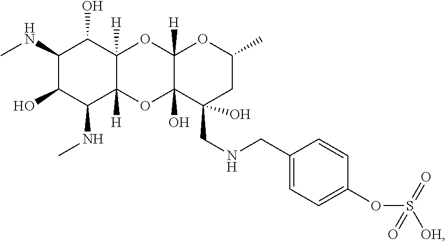 [SMILES: CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(=O)(=O)O)cc1]

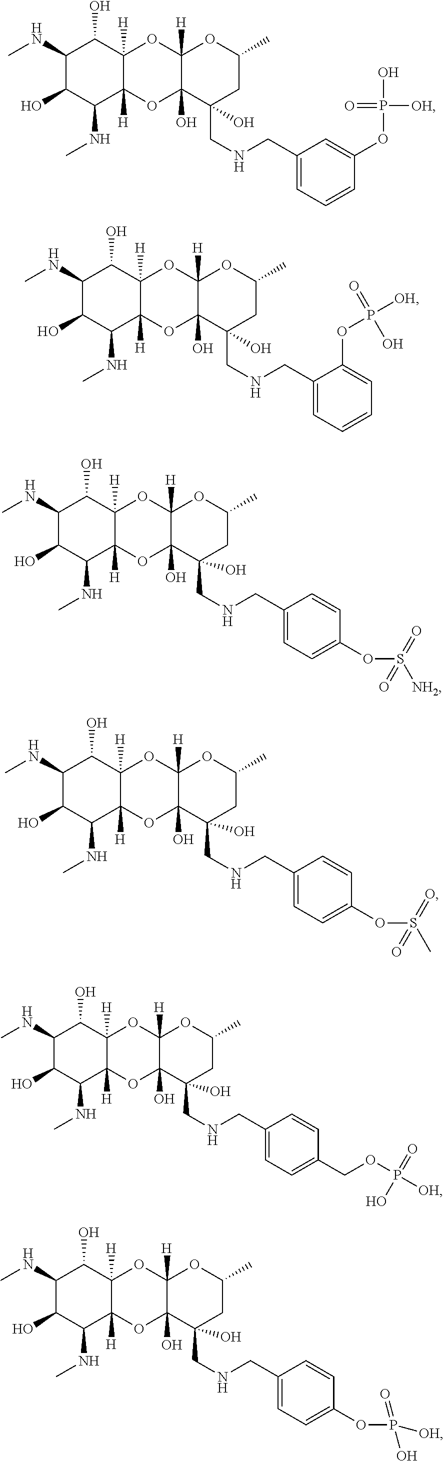

and cornbionations thereof.

In a further aspect, a disclosed compound is selected from a structure represented by a formula:

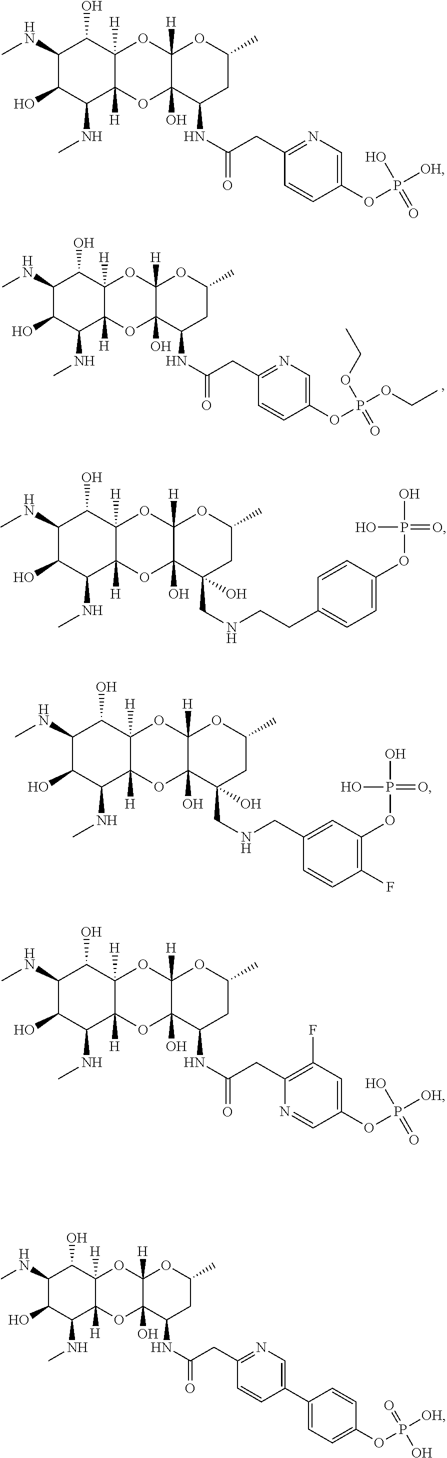

and combinations thereof.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thin, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prod. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser, 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism; A rational approach in drug design”, Chem. Rev. (Washington, D.C.) 1996, 96, 3147-3176; (ix) Soskic V, Joksimovic J, “Bioisosteric approach in the design of new dopaminergiciserotonergic ligands”, Curr. Med. Chem. 1998, 5, 493-512 (x) Thornber C W, “Isosterism and molecular modification in drug design”, Chem, Soc. Rev, 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure. Certain isotopically-labelled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present disclosure and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, e.g., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the disclosure to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e,g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzensulfonate; p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, iauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylarnine, trimethylamine, triethylarnine, ethylarnine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolarnine, diethylarnine, 2-(diethylamino)-ethanol, ethanolamine, ethylenediamine, N-methyl-glucamine, hydrabamine, L-lysine, magnesium hydroxide, 1-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### C. Methods of Making the Compounds

The compounds of this disclosure may be manufactured by the methods provided below, by the methods provided in the examples or by analogous methods. Appropriate reaction conditions for the individual reaction steps are known to a person skilled in the art. Starting materials are either commercially available or can be prepared by methods analogous to the methods given below, by methods described in references cited in the text or in the examples, or by methods known in the art. It is understood that reference to a product of a disclosed method of making a compound is inclusive of the disclosed product, as well as pharmaceutically acceptable salt, hydrate, solvate, or polymorph forms thereof.

In one aspect, the invention relates to methods of making compounds useful as antibacterial agents, which can be useful in the treatment of bacterial infections. In one aspect, the invention relates to the disclosed synthetic manipulations. In a further aspect, the disclosed compounds comprise the products of the synthetic methods described herein.

In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one product of the disclosed methods with a pharmaceutically acceptable carrier or diluent.

The compounds of this invention can be prepared by employing reactions as shown in the disclosed schemes, in addition to other standard manipulations that are known in the literature, exemplified in the experimental sections or dear to one skilled in the art. For clarity, examples having a fewer substituent can be shown where multiple substituents are allowed under the definitions disclosed herein. Thus, the following examples are provided so that the invention might be more fully understood, are illustrative only, and should not be construed as limiting.

Synthesis Scheme 1

In one aspect, a useful intermediate for the preparation of the disclosed substituted spectinoamide analogues can be prepared generically by the synthesis scheme as shown below.

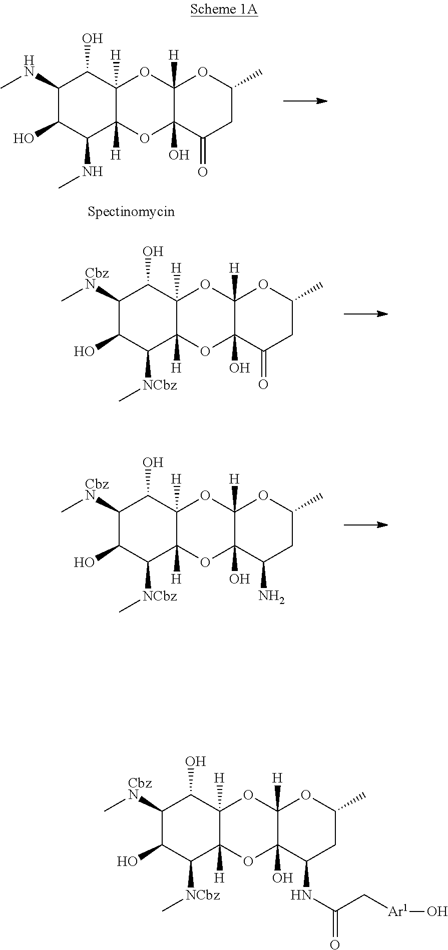

Compounds are represented in generic form, with substituents as noted in compound descriptions elsewhere herein. A more specific example is set forth below.

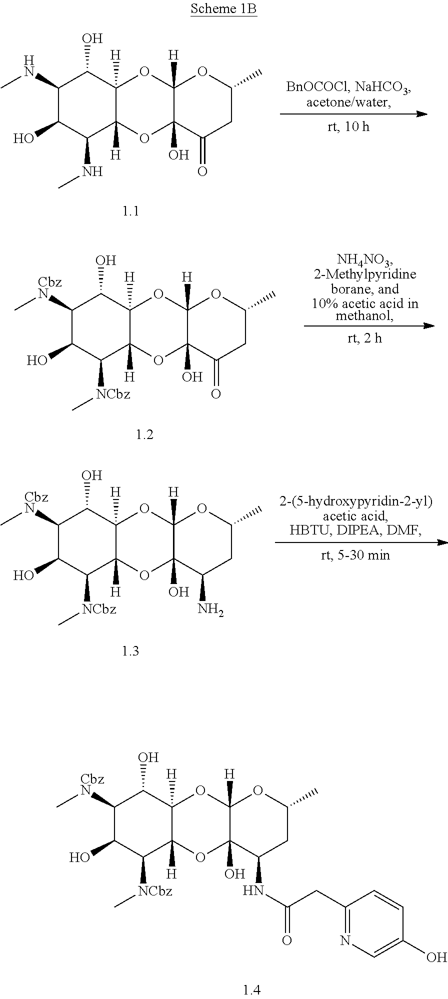

The target substituted spectinamide analogue, e.g., compound 1.4 depicted in reaction Scheme 1B above, and related compounds comprising a hydroxyl substituted aryl group, can be prepared beginning with spectinomycin, 1.1. In the intial step, the 1- and 3-aminomethyl groups are protected. The specific reaction shown above yields the CBz protected, 1.2, following reaction with benzyl chloroformate, with the reaction carried out in the presence of a suitable base, e.g. NaHCO₃, in a suitable solvent, e.g. water, and the reaction carried out a suitable temperature, e.g. about 20-30° C., for a suitable period of time, e.g. 10-18 hr, to complete the reaction. Compound 1.3 can be prepared by reaction of compound 1.2 with ammonium nitrate in the presesnce of a suitable reducing agent, e.g., 2-methylpyridine borane, in a suitable solvent, e.g 10% acetic acid in methanol, at a suitable temperature, e.g about 15° C. to about 30° C., for a suitable period of time, e.g., about 15 minutes to about 24 hours, to insure completion of the reaction. As can be appreciated by one skilled in the art, alternative conditions can be used for reductive amination of compound 1.2 to yield the desired amine derivative, compound 1.3. The target substituted spectinamide analogue, e.g., compound 1.4, can be prepared by reaction of the preceding compound, 1.3, with a suitable aryl acetic acid, e.g., 2-(5-hydroxypyridin-2-yl)acetic acid in the above reaction, under suitable conditions for amidation. As shown above, HBTU coupling can be used in presence of a suitable base, e.g., triethylamine, and suitable solvent, e.g DMF, at a suitable temperature, e.g., about 15° C. to about 30° C., for a suitable period of time, e.g., about 1 minute to about 120 minutes, to insure completion of the reaction. As can be appreciated by one skilled in the art, alternative conditions can be used for arnidation of compound 1.3 to yield the desired substituted spectinoamide analog, compound 1.4.

Synthesis Scheme 2

In one aspect, disclosed aryl substituted spectinoamide analogues comprising a disclosed phosphoester moiety can be prepared generically by the synthesis scheme as shown below.

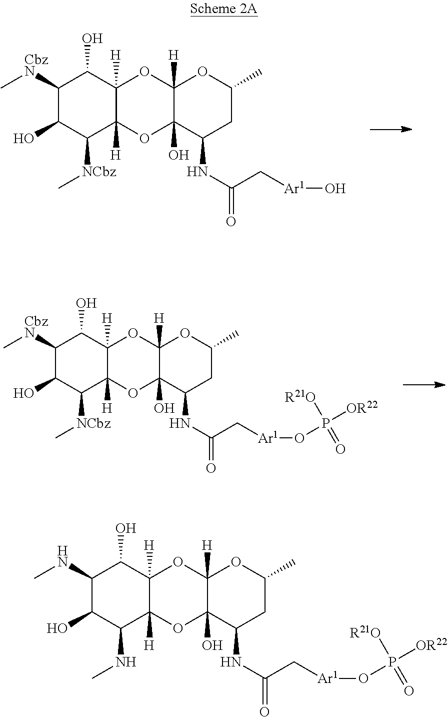

Compounds are represented in generic form, with substituents as noted in compound descriptions elsewhere herein. A more specific example is set forth below.

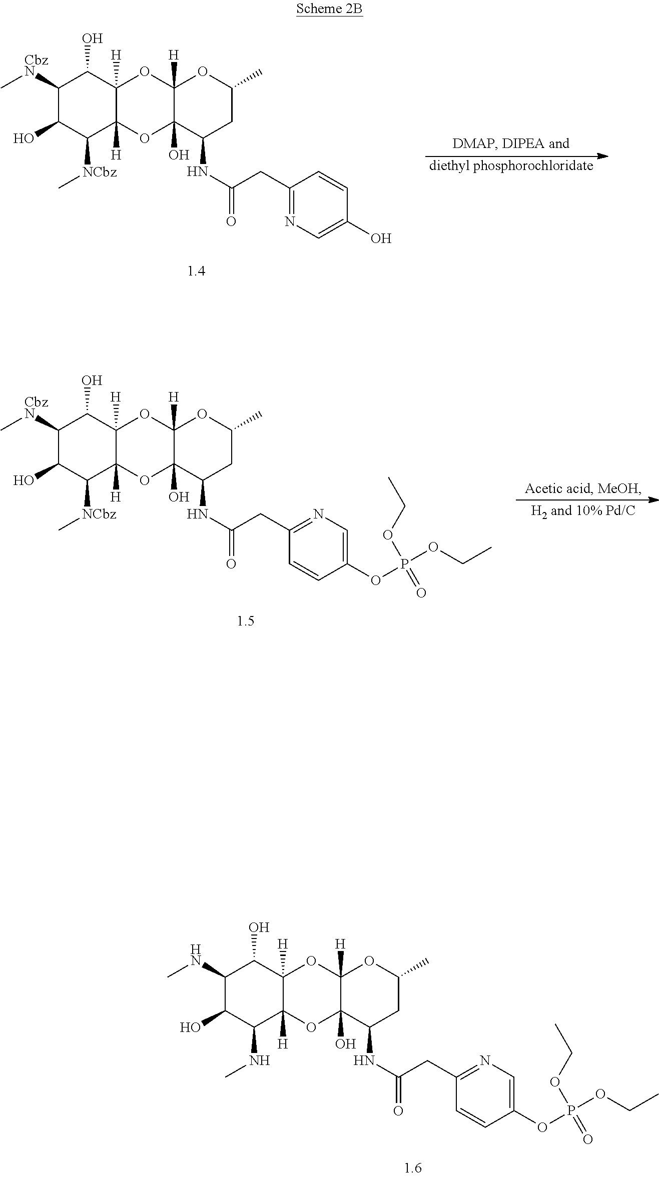

The target aryl substituted spectinoarnide analogues comprising a disclosed phosphoester moiety, e.g., compound 1.6 depicted in reaction Scheme 2B above, and related compounds can be prepared as described above, In the intial step, the hydroxyl moiety of a suitable aryl substituted spectinoamide analog, e.g., compound 1.4 above, is esterified with a desired phosphoester group using a suitable phosphoester reagent, e.g., diethyl phosphorochloridate, in the presence of a suitable catalyst, e.g., 4-dimethylaminopyridine (DMAP), and a suitable base, e.g., N,N-diisopropylethylamine (DIPEA), and the reaction carried out a suitable temperature, e.g. about 20-30° C., for a suitable period of time, e.g. about 30 minutes to about 12 hours, sufficient to complete the reaction. The amine protecting groups, i.e., CBz, can be removed by hydrogenation in the presence of hydrogen at a suitable pressure, e.g., 15-100 psi, a suitable solvent, e.g. acetic acid in methanol, in the presence of a suitable catalyst, e.g. 10% Pd/C, a suitable temperature, e.g. about 20-30° C. for a suitable period of time, e.g. about 1 hour to about 24 hours, sufficient to complete the deprotection of the amine groups.

A similar reaction to the foregoing in which the phosphoester is reduced to the acidic phosphoester form is shown below.

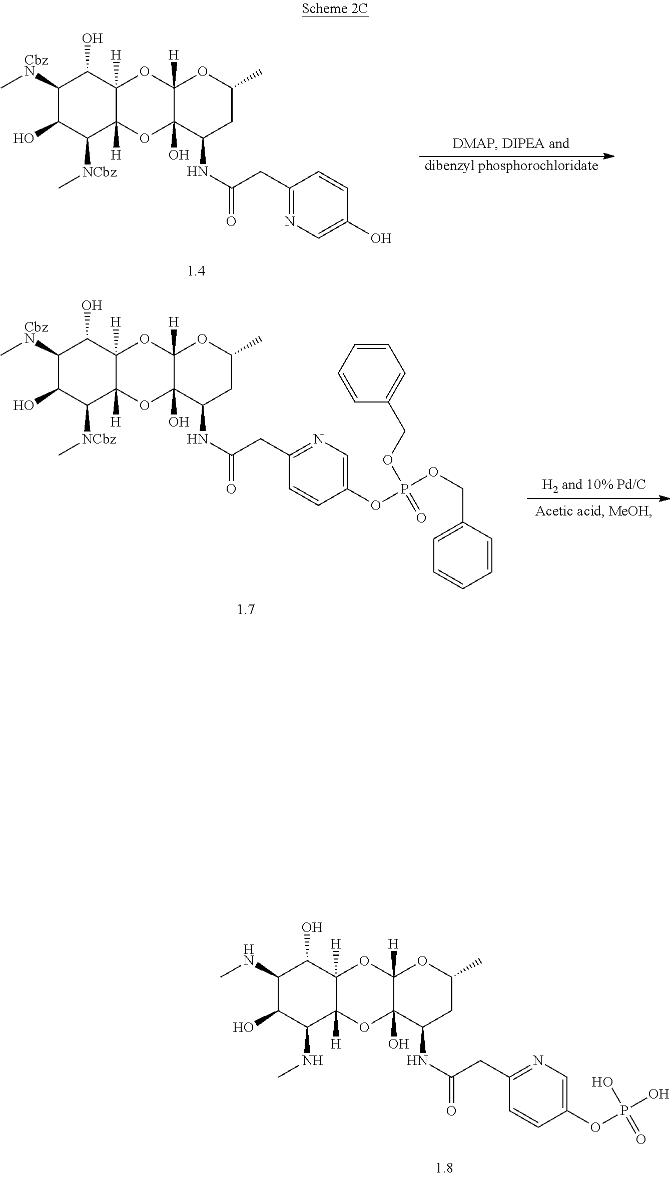

The reaction is carried out similar to the initial steps for the preceding reaction to provide the desired substituted phosphoester, compound 1.7, which is then reduced to generate the target acid phosphoester, compound 1.8. Briefly, the reduction step is carried out in the presence of a suitable hydrogenation reagent, e.g., 10 wt % Pd/C, and a suitable solvent system, e.g., acetic acid in methanol, and the reaction carried out a suitable temperature, e.g. about 20-30° C., for a suitable period of time, e.g. about 1 hour to about 24 hours, sufficient to complete the reduction of the phosphoester and removal of the amine protecting groups.

Although certain reaction conditions are provided above in Schemes 2B and 2C, other reaction conditions are possible in the initial reaction step utilizing compound 1.4 for prepartion of the phosphoester, such as compound 1.5 or 1.7, as would be known to the skilled artisan.

Exemplary compounds that can prepared by the foregoing method include:

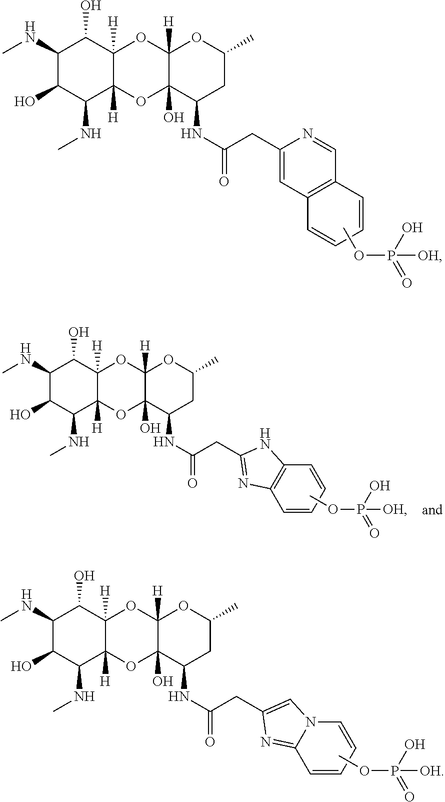

Synthesis Scheme 3

In one aspect, disclosed aryl substituted spectinoamide analogues comprising a disclosed suifoester moiety can be prepared generically by the synthesis scheme as shown below.

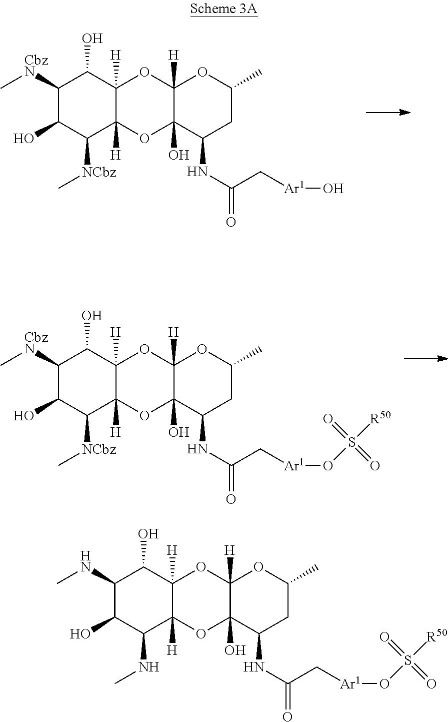

Compounds are represented in generic form, with substituents as noted in compound descriptions elsewhere herein. In the foregoing, Rs° is a group selected from R²¹, OR²¹, and NR²¹R²². A more specific example is set forth below,

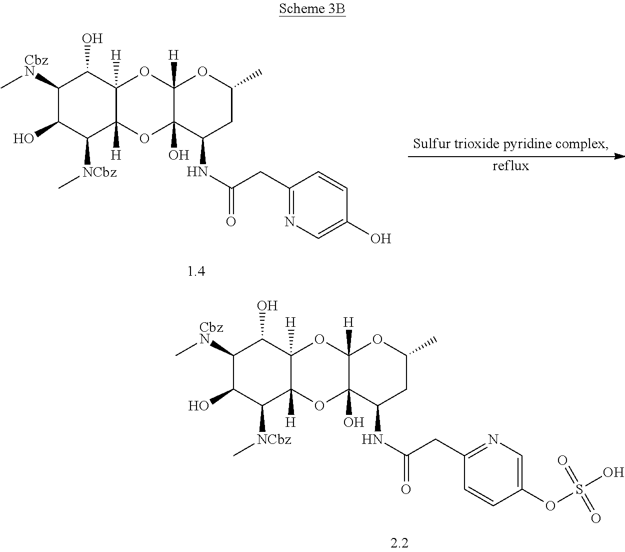 [SMILES: C[C@@H]1C[C@@H](NC(=O)Cc2ccc(O)cn2)[C@]2(O)O[C@H]3[C@H](O[C@@H]2O1)[C@@H](O)[C@H](N(C)C(=O)OCc1ccccc1)[C@H](O)[C@@H]3N(C)C(=O)OCc1ccccc1 ; "Sulfur trioxide pyridine complex," ; "reflux" ; C[C@@H]1C[C@@H](NC(=O)Cc2ccc(OS(=O)(=O)O)cn2)[C@]2(O)O[C@H]3[C@H](O[C@@H]2O1)[C@@H](O)[C@H](N(C)C(=O)OCc1ccccc1)[C@H](O)[C@@H]3N(C)C(=O)OCc1ccccc1]

The target aryl substituted spectinoarnide analogues comprising a disclosed sulfate moiety, e.g., compound 2.2, depicted in reaction Scheme 3B above, and related compounds can be prepared as described above. Briefly, the hydroxyl moiety of a suitable aryl substituted spectinoamide analog, e.g., compound 1.4 above, is sulfated with a desired sulfation reagent, e.g., sulfur trioxide pyridine complex, under suitable conditions to effect sulfation, e.g., refluxing for about 1-6 hours, to achieve sufficient sulfation of the target hydroxy gorup. Other sulfate derivates, e.g., R⁵⁰ is —R²¹ or —NR²¹R²² can be prepared by similar methods as known to one skilled in the art.

Although certain reaction conditions are provided above in Scheme 3B, other reaction conditions are possible in the initial reaction step utilizing compound 1.4 to prepare a derivative with a sulfate moiety, such as compound 2.2. For example, reaction of compound 1.4 with methanesulfonyl chloride in the presence of a suitable catalyst, e.g., 4-dimethylaminopyridine (DHAP), and a suitable base, e.g., N,N-diisopropylethylarnine (DIPEA), in a suitable solvent, e.g., dichloromethane (DOM) and the reaction carried out a suitable temperature, e.g. about 20-30° C., for a suitable period of time, e.g, about 30 minutes to about 12 hours, sufficient to complete the reaction. Alternatively, reaction of compound 1.4 can be carried out with sodium hydride and sulfamoyl chloride is possible. In some instances, such reaction conditions will yield an intermediate with an alkyl or aryl group which can be removed under reducing conditions. For example, the reduction step can be carried out as described above, in the presence of a suitable hydrogenation reagent, e.g., 10 wt % Pd/C, and a suitable solvent system, e.g., acetic acid in methanol, and the reaction carried out a suitable temperature, e.g. about 20-30° C., for a suitable period of time, e.g. about 1 hour to about 24 hours, sufficient to complete the reduction of the phosphoester and removal of the amine protecting groups.

Exemplary compounds that can prepared by the foregoing method include:

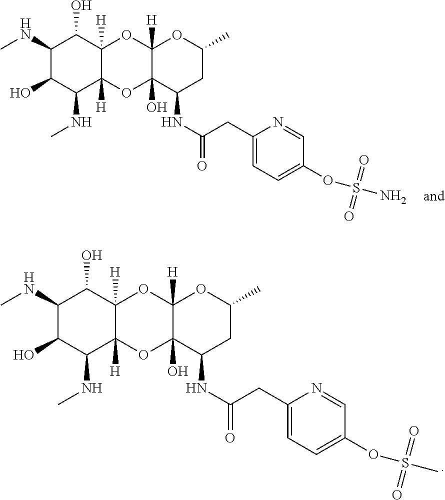

Synthesis Scheme 4

In one aspect, disclosed aryl substituted aminomethyl spectinomycin analogues comprising a disclosed phosphoester moiety can be prepared generically by the synthesis scheme as shown below.

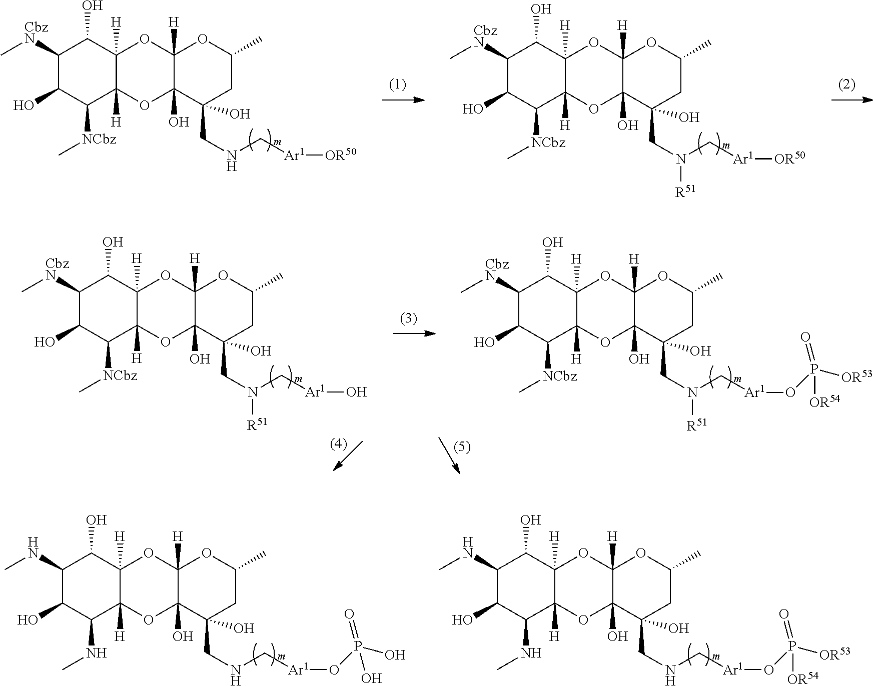

Briefly, the first compound in the foregoing synthesis scheme can be prepared using the methods disclosed herein throughout. The synthesis scheme can utilize the following exemplary reaction conditions (which can be further modified or substituted with alternative conditions as known to the skilled artisan): (1) Cbz protection (BnOCOCl, NaHCO₃, acetone/water, room temperature; 10 h) or Boc protection (Di-tert-butyl dicarbonate, MeOH, Et₃N, room temperature; 1-4 h); (2) Boc deprotection, 4 M HCl in dioxane or Trifluoroacetic acid, room temperature, 1 h, or ammonium hydroxide aqueous in MeOH, room temperature, 5 h; (3) DMAP, DIPEA and dibenzyl (or dimethyl) phosphorochloridate, room temperature, 1-4 h; (4) a. Boc deprotection (4 M HCl in dioxane or Trifluoroacetic acid, room temperature, 1 h), b. Acetic acid or HCl, MeOH, H₂ and 10% Pd/C room temperature 1-24 h; and (5) a, Boc deprotection (4 M HCl in dioxane or Trifluoroacetic acid, room temperature, 1 h), b. Acetic acid or HCl, MeOH, H₂ and 10% Pd/C room temperature 1-24 h, In the foregoing, unless otherwise specified, substituent groups have the defined scope as disclosed herein. The following R groups are further defined: R⁵⁰ is a Boc or other suitable hydroxyl protecting, e.g., MOM; R⁵¹ is a Cbz, Boc or other suitable amine protecting group; and each of R⁵³ and R⁵⁴ are a C1-C6 alkyl, Cbz, or Boc group.

Exemplary compounds that can prepared by the foregoing method include;

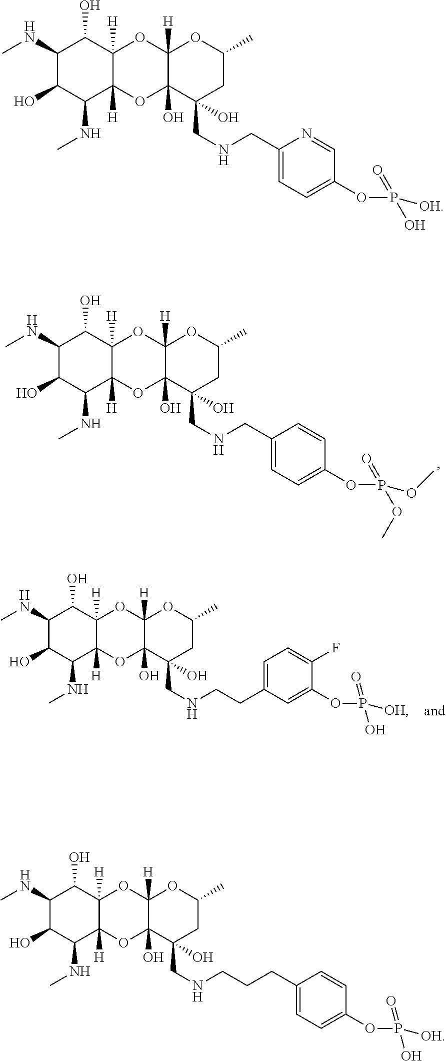

Synthesis Scheme 5

In one aspect, disclosed aryl substituted aminomethyl spectinomycin analogues comprising a disclosed sulfoester moiety can be prepared generically by the synthesis scheme as shown below.

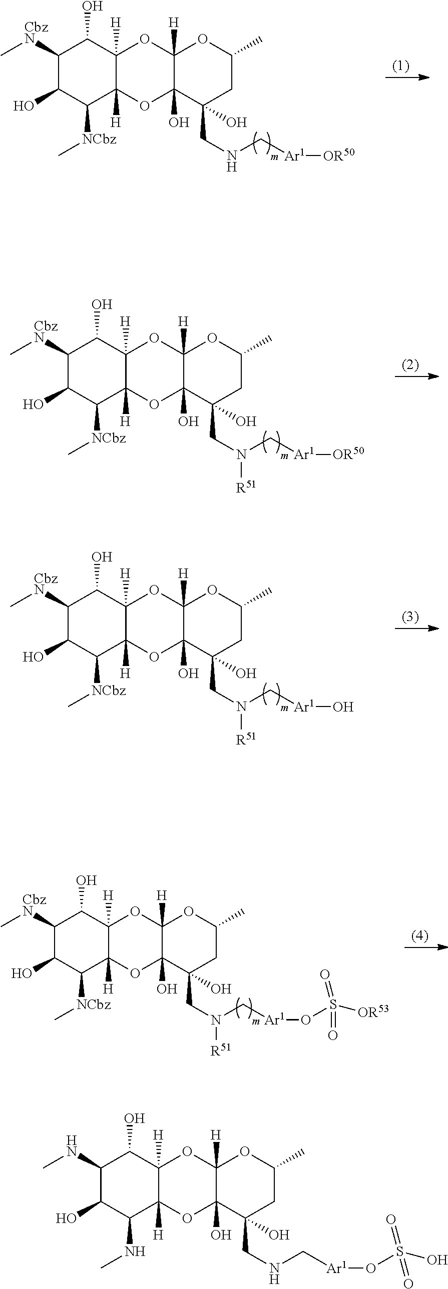

Briefly, the first compound in the foregoing synthesis scheme can be prepared using the methods disclosed herein throughout. The synthesis scheme can utilize the following exemplary reaction conditions (which can be further modified or substituted with alternative conditions as known to the skilled artisan): (1) Cbz protection (BnOCOCl, NaHCO₃, acetone/water, room temperature; 10 h) or Boc protection (Di-tert-butyl dicarbonate, MeOH, Et₃N, room temperature; 1-4 h); (2) Boc deprotection, 4 M HCl in dioxane or Trifluoroacetic acid, room temperature, 1 h, or ammonium hydroxide aqueous in MeOH, room temperature, 5 h; (3) Sulfur trioxide pyridine complex, pyridine, refluxing, 4 h; or 2,2,2-Trichloroethyl chlorosulfate, DIPEA, DMAP, DCM, room temperature, 12 h, or methanesulfonyl chloride, DIPEA, DMAP, DCM, room temperature, 2 h; or sodium hydride, sulfamoyl chloride; and (4) Acetic acid or HCl, MeOH, H₂ and 10% Pd/C, room temperature, 1-24 h. The following R groups are further defined: R^{SC} is a Boc or other suitable hydroxyl protecting, e.g., MOM; R⁵¹ is a Cbz, Boc or other suitable amine protecting group; and R⁵³ is a C1-C6 alkyl, Cbz, or Boc group.

Exemplary compounds that can prepared by the foregoing method include:

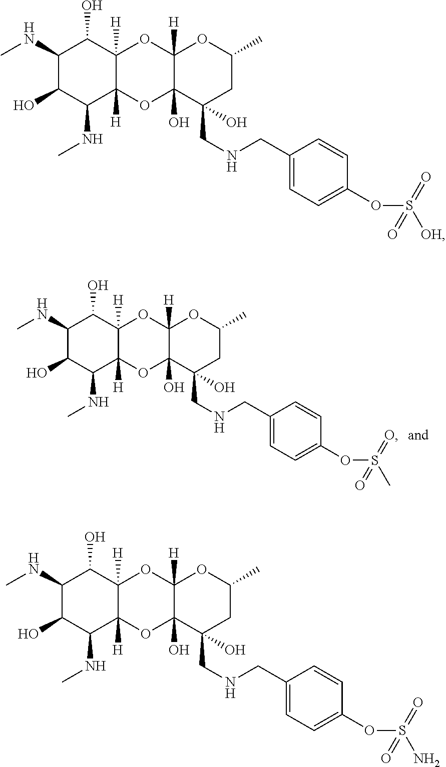 [SMILES: CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(=O)(=O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(C)(=O)=O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(N)(=O)=O)cc1]

using the following reaction conditions for step (3) in the general reaction above for the indicated compound, respectively: (a) sulfur trioxide pyridine complex, pyridine, refluxing, 4 h; or 2,2,2-Trichloroethyl chlorosulfate, DIPEA, DMAP, DCM, room temperature, 12 h; (b) methanesulfonyl chloride, DIPEA, DMAP, DCM, room temperature, 2 h; and (c) sodium hydride and sulfamoyl chloride.

Synthesis Scheme 6

In one aspect, an exemplary disclosed aryl substituted spectinoamide analogue comprising a disclosed (C1-C3)-alkanediyl-OP(O)(OR²¹) moiety can be prepared by the synthesis scheme as shown below.

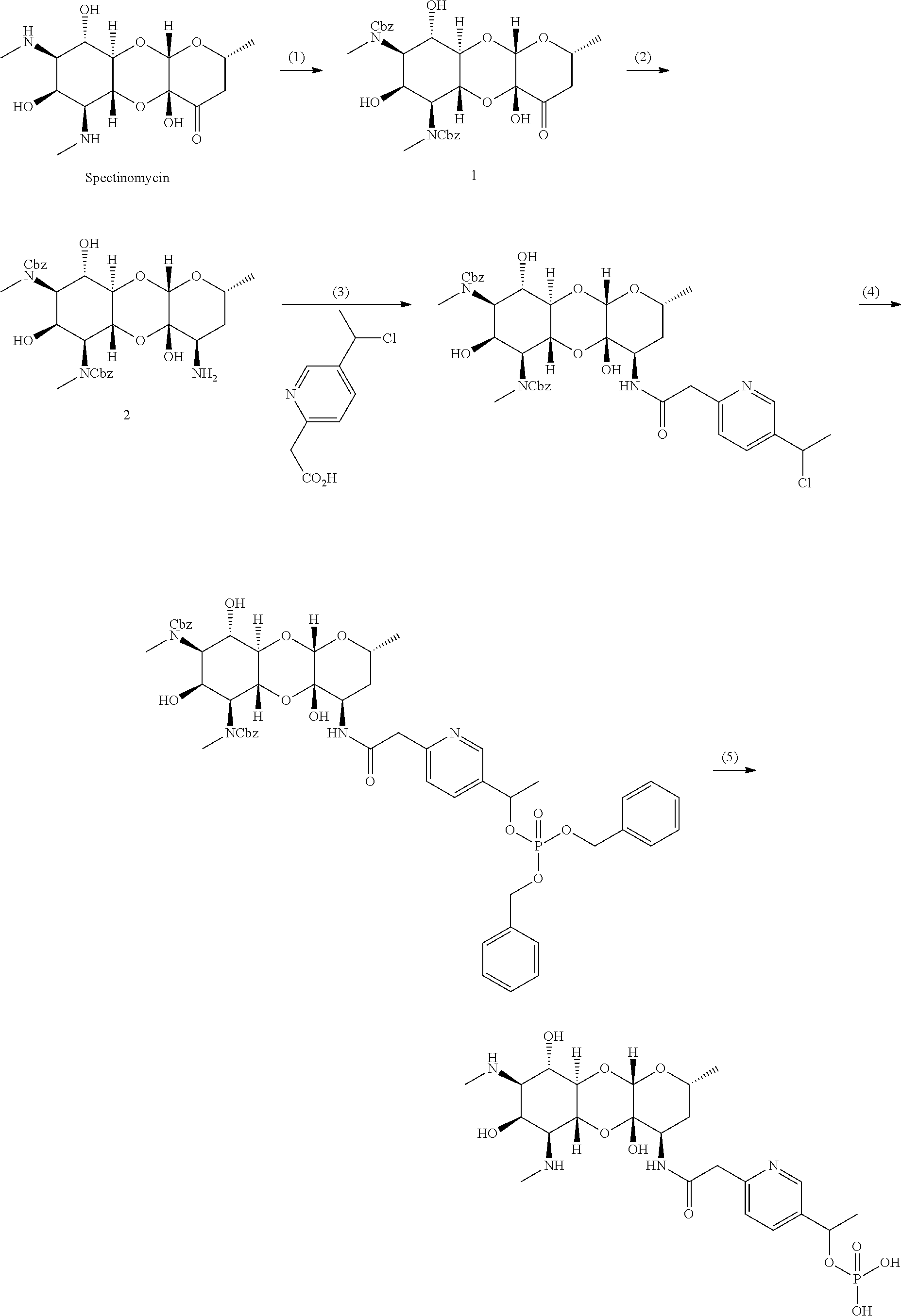

The foregoing synthesis scheme can utilize the following exemplary reaction conditions (which can be further modified or substituted with alternative conditions as known to the skilled artisan): (1) BnOCOCl, NaHCO₃, acetone/water, room temperature, 10 h, 90%; (2) NH₄NO₃, 2-methyipyridine borane, 10% acetic acid in methanol, room temperature, 2 h; (3) HBTU, DIPEA, DMF, room temperature, 1 h; (4) silver(I) dibenzyl phosphate, toiune, reluxing; 2 h; and (5) H₂, 10% Pd/C, HOAc or HCl in MeOH, room temperature, 1-24 h.

Synthesis Scheme 7

In one aspect, an exemplary disclosed aryl substituted aminomethyl spectinomycin analogue comprising a disclosed phosphoester moiety can be prepared generically by the synthesis scheme as shown below.

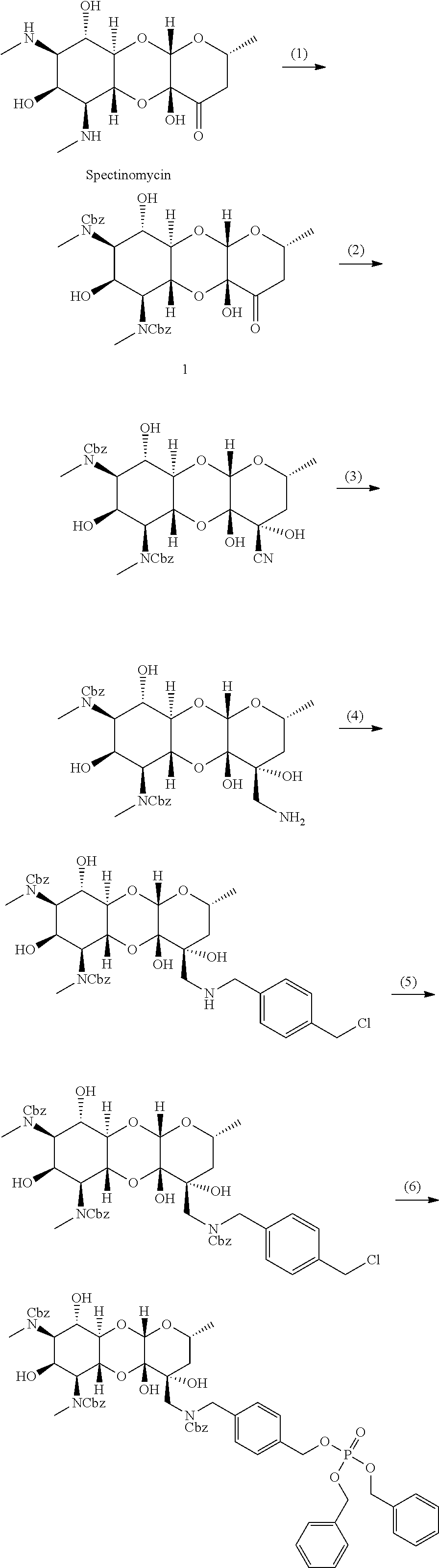

The foregoing synthesis scheme can utilize the following exemplary reaction conditions (which can be further modified or substituted with alternative conditions as known to the skilled artisan): (1) BnOCOCl, NaHCO₃, acetone/water, room temperature; 10 h, 90%; (2) KCN, MeOH—H₂O, 1N AcOH, RT. room temperature, 4 h; (3) H2, Raney Ni, AcOH, MeOH, RT, room teniperature,10 h; (4) aldehyde, 2-picoline borane, AcOH-MeOH (1:10), room temperature, 1-12 h; (5) BnOCOCl, NaHCO₃, acetone/water, room temperature; 10 h; (6) silver(I) dibenzyl phosphate, tolune, reluxing, 2 h; and (7) H2, 10% Pd/C, HOAc or HCl in MeOH, room temperature, 1-24 h.

### D. Pharmaceutical Compositions

In various aspects, the present disclosure relates to pharmaceutical compositions comprising a therapeutically effective amount of a disclosed compound, or a pharmaceutically acceptable salt thereof, and a pharmaceutically acceptable carrier. In a further aspect, the present disclosure relates to pharmaceutical compositions comprising a therapeutically effective amount of at least one disclosed compound or at least one disclosed product of a method of making a compound, as well as pharmaceutically acceptable salt, hydrate, solvate, or polymorph forms of the disclosed compound or the disclosed product of a method of making compound.

As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucarnine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylarnine, ethylamine, propylamine, isopropylamine, any of the four butylarnine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylarninoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, caraphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes. Editors, 1979); Pharmaceutical Dosage Forms: Tablets (Lieberman et al:, 1981); Ansel, Introduction to Pharmaceutical Dosage Forms 2nd Edition (1976); Remington's Pharmaceutical Sciences, 17th ed. (Mack Publishing Company, Easton, Pa, 1985); Advances in Pharmaceutical Sciences (David Ganderton, Trevor Jones, Eds., 1992); Advances in Pharmaceutical Sciences Vol 7. (David Ganderton,

Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols. dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc. starch, gelatin, agar, pectin, acacia, magnesium stearate, and stearic acid, Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate. magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985), Various enteric coating materials may be selected on the basis of testing to achieve an enteric coated dosage form designed ab initio to have a preferable combination of dissolution time, coating thicknesses and diametral crushing strength (e,g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate,

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil). paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethypimidazolidone-(2). In this context, polyoxyethylated means that the substances in question contain polyoxyethylene chains, the degree of polymerization of which generally lies between 2 and 40 and in particular between 10 and 20, Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe fir Pharmazie, Kostnetik and angrenzende Gebiete” 1971, pages 191-195,

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascet c acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles, Liposomes can be formed from a variety of phospholipids, such as cholesterol, stearylarnine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcelluk)se. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present_{;} such as, for example, antimicrobials, antioxidants_{;} collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e,g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed Easton, Pa.: Mack Publishing Co, (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark CarbopolTM. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin, In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present disclosure include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Another patch system configuration which can be used by the present disclosure is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present disclosure is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0,05 to 99% by weight, preferably from C.1 to 70% by weight, more preferably from C.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require of inhibiting protein synthesis in a bacterial cell an appropriate dosage level will generally be about C.01 to 1000 mg per kg patient body weight per day and can be administered in single or multiple doses. In various aspects, the dosage level will be about C.1 to about 500 mg/kg per day, about C.1 to 250 mg/kg per day, or about 0,5 to 100 mg/kg per day. A suitable dosage level can be about C.01 to 1000 mg/kg per day, about 0,01 to 500 mg/kg per day, about C.01 to 250 mg/kg per day, about C.05 to 100 mg/kg per day, or about C.1 to 50 mg/kg per day. Within this range the dosage can be C.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800. 900 and 1000 mg of the active ingredient for the symptomatic adjustment of the dosage of the patient to be treated. The compound can be administered on a regimen of 1 to 4 times per day, preferably once or twice per day, This dosing regimen can be adjusted to provide the optimal therapeutic response.

Such unit doses as described hereinabove and hereinafter can be administered more than once a day, for example, 2, 3, 4. 5 or 6 times a day. In various aspects, such unit doses can be administered 1 or 2 times per day, so that the total dosage for a 70 kg adult is in the range of C.001 to about 15 mg per kg weight of subject per administration. In a further aspect, dosage is C.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 nig tablet or 1 nig to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for inhibiting protein synthesis in a bacterial cell (e.g., treatment of one or more infectious diseases associated with a bacterial infection) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an anti-microbial agent. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent that has anti-microbial activity, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### E. Methods of Using the Compounds

In various aspects, the present disclosure provides methods of treating an infectious disease comprising administration of a therapeutically effective amount of a disclosed compound, a product of a disclosed method of making a compound, or a disclosed pharmaceutical composition to a subject in need thereof. It is understood that reference to a disclosed compound is inclusive of the disclosed compound, as well as pharmaceutically acceptable salt, hydrate, solvate, or polymorph forms thereof; reference to a product of a disclosed method of making a compound is inclusive of the disclosed product, as well as pharmaceutically acceptable salt, hydrate, solvate, or polymorph forms thereof; and reference to a disclosed pharmaceutical composition is inclusive of a pharmaceutical composition comprising a disclosed compound or a disclosed product of a method of making a compound, as well as pharmaceutically acceptable salt, hydrate, solvate, or polymorph forms of a disclosed compound or a disclosed product of a method of making compound,

It is understood that treating an infectious is inclusive of treating, preventing, ameliorating, controlling or reducing the risk of a variety of bacterial infections, including an infection associated with Gram positive bacteria, Gram negative bacteria, or mycobacteria, wherein the patient or subject would benefit from an antibacterial agent. For example, a treatment can include inhibiting protein synthesis activity in bacteria by binding to bacterial ribosomes. In one aspect, provided is a method of treating or preventing a bacterial infection in a subject comprising the step of administering to the subject at least one disclosed compound; at least one disclosed pharmaceutical composition; and/or at least one disclosed product in a dosage and amount effective to treat the disorder in the subject,

Also provided is a method for the treatment of one or more disorders associated with infection by a pathogenic bacteria wherein inhibiting bacterial protein synthesis can sterilize or decrease the presence of the pathogenic bacteria in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more bacterial infections in a subject comprising the step of administering to the subject at least one disclosed compound; at least one disclosed pharmaceutical composition; and/or at least one disclosed product in a dosage and amount effective to treat the disorder in the subject. In some aspects, the subject is a human subject.

In a further aspect, the disclosure relates to a method for the treatment of an infectious disease in a human subject, further comprising the step of identifying a human subject in need of treatment of the infectious diesease.

In a further aspect, the disclosure relates to a method for the treatment of an infectious disease in a human subject comprising the step of administering to the human subject a therapeutically effective amount of at least one disclosed compound; or a pharmaceutically acceptable salt, hydrate, solvate, or polymorph thereof; wherein the compound is formulated as a lotion, a cream, an ointment, a spray, or a soap.

In a further aspect, the compound is formulated as a solid dosage form. In a still further aspect, the solid dosage form is selected from a capsule, a tablet, a pill, a powder, a granule, an effervescing granule, a gel, a paste, a troche, and a pastille. In yet a further aspect, the solid dosage form is formulated for oral administration.

In a further aspect, the compound is formulated as a liquid dosage form. In a still further aspect, the liquid dosage form is selected from an emulsion, a solution, a suspension, a syrup, and an elixir. In yet a further aspect, the liquid dosage form is formulated for intravenous administration or parenteral administration.

Also provided is a method for the treatment of a bacterial infection in a vertebrate animal comprising the step of administering to the mammal at least one disclosed compound, composition, or medicament, In some aspects, the vertebrate animal is a mammal.

In a further aspect, the vertebrate animal is a fish, a bird, or a mammal. In a still further aspect, the vertebrate animal is a livestock animal. In yet a further aspect, the vertebrate animal is a companion animal. In an even further aspect, the vertebrate animal is a farm animal. In a still further aspect, the vertebrate animal is a zoo animal. In yet a further aspect, the vertebrate animal is a laboratory animal. In an even further aspect, the vertebrate animal is an aquaculture fish. In a still further aspect, the vertebrate animal is selected from Bison sp., Bos sp., Canis sp., Capra sp., Equus sp., Fells sp., Gallus sp., Lama sp., Meleagris sp., Oryctolagus sp., Ovis sp., and Sus sp.

In a further aspect, the vertebrate animal has been diagnosed with a need for treatment of the infectious disease prior to the administering step.

In a further aspect, the disclosure relates to a method for the treatment of an infectious disease in a vertebrate animal, further comprising the step of identifying a vertebrate animal in need of treatment of the infectious diesease.

In a further aspect, administering comprises mixing an effective amount of the compound with the food of the vertebrate animal. In a still further aspect, administering comprises administering enterally an effective amount of the compound with the food of the vertebrate animal, In yet a further aspect, administering comprises administering an oral bolus of an effective amount of the compound with the food of the vertebrate animal.

In various aspects, administering to a vertebrate animal comprises intravenous administration or parenteral administration to the vertebrate animal.

In a further aspect, the infectious disease treated in the vertebrate animal is selected from dental infection, dermatitis, diarrhea, ear infection, gastritis, gastroenteritis, genitourinary infection, intestinal infection, lung infection, ocular infection, oral infection, otitis, osteo-articular infection, pharyngitis, papules, pneumonia conjunctivitis, pruritius, pustules, pyoderma, pyothorax, respiratory infection, salmonellosis, septicemia, skin infection, soft tissue infection, ulcer, urinary tract infection, and wound infection.

In a further aspect, the disclosure relates to a method for the treatment of an infectious disease in a vertebrate animal, further comprising administering to the vertebrate animal a therapeutically effective amount of second active agent. In a still further aspect, the second active agent is an antibacterial agent. In yet a further aspect, the antibacterial agent is pencillin, a cephalosporin, a sulfonamide, a tetracycline, a lincosamide, an aminoglycoside, or a fluoroquinolone, or combinations thereof, In an even further aspect, the antibacterial agent comprises a compound selected from amoxicillin, ampicillin, azithromycin, cefovecin, cephalexin, chloramphenicol, ciprofloxacin, clavulanic acid, cloxacillin, clindamycin, doxycycline, enrofloxacin, erythromycin, gentarnicin, ibafloxacin, kanamycin, lincomycin, marbofloxacin, metronidazole, minocycline, neomycin, novobiocin. ofloxacin, orbifloxacin, oxytetracycline, penicillin G, rifampin, sulfadimethoxine, sulfadiazine, tetracycline, tiarnulin, ticarcillin, trimethoprim, and tylosin, or combinations thereof.

The compounds are further useful in a method for the prevention, treatment, control, amelioration, or reduction of risk of the bacterial infections noted herein. The compounds are further useful in a method for the prevention, treatment, control, amelioration, or reduction of risk of the aforementioned bacterial infections in combination with other agents.

In one aspect, the disclosed compounds can be used in combination with one or more other drugs in the treatment, prevention, control, amelioration, or reduction of risk of bacterial infections for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present disclosure. When a compound of the present disclosure is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present disclosure.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the bacterial infections for which disclosed compounds are useful. Such other drugs can be administered, by a route and in an amount commonly used therefor, contemporaneously or sequentially with a compound of the present disclosure. When a compound of the present disclosure is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present disclosure.

The weight ratio of a disclosed compound to the second active ingredient can be varied and will depend upon the effective dose of each ingredient. Generally, an effective dose of each will be used. Thus, for example, when a compound of the present disclosure is combined with another agent, the weight ratio of a disclosed compound to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s),

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In one aspect, the compound can be employed in combination with antibacterial or antimicrobial agents, and combinations thereof, and the like, or the subject compound can be administered in conjunction with the use of physical methods such as with debridement of a wound or infected tissue.

In the treatment of an infectious disease condition, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about C.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one aspect, the disclosure relates to methods for treating a bacterial infection in at least one cell, comprising the step of contacting the at least one cell with at least one compound of the disclosure, in an amount effective to alter the response in the at least one cell. In a further aspect, the cell is mammalian, for example human. In a further aspect, the cell has been isolated from a subject prior to the contacting step. In a further aspect, contacting is via administration to a subject.

Infectious diseases treatable by the presently disclosed compounds can be caused by a variety of bacteria and protozoa. In some embodiments, the infection is a bacterial infection. Exemplary microbial infections that can be treated by the method of the presently disclosed compounds include, but are not limited to, infections caused by Staphylococcus aureaus, Enterococcus faecalis, Bacillus anthracis, a Streptococcus species (e.g., Streptococcus pyogenes and Streptococcus pneumoniae), Escherichia Pseudomonas aeruginosa, Burkholderia cepacia, a Proteus species (e.g., Proteus mirabilis and Proteus vulgaris), Klebsiella pneumoniae, Acinetobacter baumannii, Strenotrophomonas maltophillia, Mycobacterium tuberculosis, Mycobacterium bovis, other mycobacteria of the tuberculosis complex, and non-tuberculous mycobacteria, including Mycobacterium ulcerans, Mycobacterium avium and Mycobacterium abscessus,

An infectious disease that is associated world-wide with a high level of morbidity and mortality is a mycobaterial infection. Mycobacterial infections can cause different diseases such as tuberculosis (“TB”). Additionally, mycobacterial diseases can cause overwhelming, disseminated disease in immunocompromised patients and is the leading killer of people who are HIV infected. In spite of the efforts of numerous health organizations worldwide, the eradication of mycobacterial diseases has never been achieved, nor is eradication imminent. Based on currently available data, about one fourth of the world's population is infected with TB (CDC data; see https://www.cdc.govitb/statisticsidefaulthtrn; accessed on Oct. 10, 2018). Moreover, in 2016, 10.4 million people around the world became sick with TB disease and there were 1.7 million TB-related deaths worldwide (CDC data op. cit.).

Although over 37 species of Mycobacterium have been identified, more than 95% of all human infections are caused by seven species of mycobacteria: M. tuberculosis, M. avium intracellulare. M. abscessus. M, kansasii, M, fortuiturn, M. chelonae, and M. leprae. Cases of human tuberculosis are predominantly caused by mycobacterial species comprising M. tuberculosis, M. bovis, or M. africanum. Infection is typically initiated by the inhalation of infectious particles, which are able to reach the terminal pathways in the lungs. Following engulfment by alveolar macrophages, the bacilli are able to replicate freely, with eventual destruction of the phagocytic cells. A cascade effect ensues wherein destruction of the phagocytic cells causes additional macrophages and lymphocytes to migrate to the site of infection, where they too are ultimately eliminated.

Mycobacteria can be classified into several major groups for purpose of diagnosis and treatment: M. tuberculosis complex (MTBC), which can cause tuberculosis (M. tuberculosis, M. bovis, M, africanum, and M. microti); M. leprae, which causes Hansen's disease or leprosy; and Nontuberculous mycobacteria (NTM) are all the other mycobacteria, which can cause pulmonary disease resembling tuberculosis, lymphadenitis, skin disease, or disseminated disease. MTBC members are causative agents of human and animal tuberculosis. Species in this complex include: M. tuberculosis, the major cause of human tuberculosis, M. bovis, M. bovis BOG, M. africanum, M. canetti, M. caprae, M. microti, and M. pinnipedii.

In a further aspect, the present disclosure provides methods of treating a mycobacterial infections, including those caused by mycobacteria such as M. tuberculosis, M. bovis, M. bovis BCG, M. africanurn, M. canetti, M. caprae, M. microti, M. pinnipedii, M. avium, M. avium paratuberculosis, M. avium silvaticum, M. avium “homninissuis”, M. colombiense, M. asiaticum, M. gordonae, M. gastri, M. kansasii, M. hiberniae, M. nonchromogenicum, M. terrae, Al, triviale, Al. ulcerans, M. pseudoshottsii, M. shottsii, M. triplex, M. genavense, M. florentinum, M. lentiflavum, M. palustre, M. kubicae, M. parascrofulaceum, M. heidelbergense, M. interjectum, M. sirniae, M. branderi, M. cookii, M. celaturn, M. bohernicum, M. haemophilum, M. mairnoense, M. szufgai, M. leprae, M. lepraemurium, M. lepromatosis, M. botniense, M. chimaera, M. conspicuurn, M. doricum, M. farcinogenes, M. heckeshornense, M. intracellulare, M. lacus, M. marinum, M. monacense, M. montefiorense, M. murale, M. nebraskense, M. saskatchewanense, M. scrofulaceum, M. shimoidei, M. tusciae, M. xenopi, M. intermedium, M. abscesses, M. chelonae, M. bolletii, M. fortuitum, M. fortuitum subsp. acetamidolyticum, M. boenickel, M. peregrinum, M. porcinurn, M. senegalense, M. septicum, M. neworleansense, M. houstonense, M. mucogenicum, M. mageritense, M. brisbanense, M. cosrneticum, M. parafortuiturn, M. austroafricanum, M. diemhoferi, M. hodieri, M. neoaurum, M. frederiksbergense. M. aurum, M. vaccae, M. chitae, M. fallax, M. confluentis, Al, flavescens, M. madagascariense, M. phlei, M. smnegmatis, M. goodii, M. wolinskyi, M. thermoresistibile, M. gadium, M. komossense, M. obuense, M. sphagni, M. agri, M. aichiense, M. alvei, M. arupense, M. brumae, M. canariasense, M. chubuense, M. conceptionense, M. duvalii, M. elephantis, M. gifvum, M. hassiacum, M. holsaticum, M. immunogenum, M. massiliense, M. moriokaense, N. psychrotolerans, pyrenivorans, M. vanbaalenii, M. pulveris, M. arosiense, M. aubagnense, M. caprae, M. chlorophenolicum, M. fluoroanthenivorans, M. kumamotonense, M. novocastrense, M. parrnense, M. phocaicum, M. Poriferae, M. rhodesiae, M. seoulense, and M. tokaiense.

In a further aspect, the present disclosure provides methods of treating an infectious disease such as a mycobacterial infection. In various aspects, the mycobacterial infection can be associated with a Mycobacterium tuberculosis infection. In a still further aspect, the Mycobacterium tuberculosis infection is associated with infection by an MDR strain of Mycobacterium tuberculosis. In a yet further aspect, the Mycobacterium tuberculosis infection is associated with infection by an XDR strain of Mycobacterium tuberculosis.

In a further aspect, the present disclosure provides methods of treating an infectious disease such as a Gram positive bacterial infection. In a still further aspect, the Gram positive bacteria is selected from Bacillus sp. Clostridium sp., Corynebacterium sp, Enterococcus sp., Mycoplasma sp., Staphylococcus sp., and Streptococcus sp. In yet a further aspect, the Gram positive bacteria is vancomycin resistant Enterococcus sp. (VRE). In an even further aspect, the Gram positive bacteria is methicillin resistant Staphylococcus sp. (MRS). In a still further aspect, the Gram positive bacteria is selected from Bacillus anthracis, Bacillus cereus, Bacillus subtilis, Clostridium difficile, Clostridium tetani, Clostridium botulinum, Clostridium perfringens, Corynebacterium diphtheria, Enterococcus faecalis, Enterococcus faecium, Listeria monocytogenes, Listeria ivanovii, Micrococcus luteus, Mycoplasma genitalium, Mycoplasma pneumoniae, Staphylococcus aureus, Staphylococcus epidermidis, Staphylococcus saprophyticus, Staphylococcus hyicus, Staphylococcus intermedius, Streptococcus pneumoniae, and Streptococcus pyogenes. In yet a further aspect, the Gram positive bacteria is selected from Bacillus anthracis, Bacillus subtilis, Enterococcus faecalis, Staphylococcus aureus, Streptococcus pneumoniae, and Streptococcus pyogenes. In an even further aspect, the Gram positive bacteria is selected from vancomycin resistant Enterococcus faecalis, vancomycin resistant methicillin resistant Enterococcus faecium, Staphylococcus aureus (MRSA), methicillin resistant Staphylococcus epidermidis (MRSE), macrolide resistant Streptococcus pneumoniae (Mac-R SPN) and penicillin resistant Streptococcus pneumonia (PRSP).

In a further aspect, the present disclosure provides methods of treating an infectious disease such as a Gram negative bacterial infection. In a still further aspect, the Gram negative bacteria is selected from Acinetobacter sp., Aeromonas sp., Burkholderia sp., Bordatella sp., Citrobacter sp., Chlamydia sp., Enterobacter sp., Escherichia sp., Francisella sp., Haemophilus sp., Klebsiella sp., Legionella sp., Moraxella sp., Neisseria sp., Proteus sp., Pseudomonas sp., Rickettsia sp., Salmonella sp., Shigella sp., Stenotrophomonas sp., Vibrio sp., and Yersinia sp. in yet a further aspect, the Gram negative bacteria is selected from Acinetobacter baumannii, Aeromonas hydrophile, Bordetella pertussis, Bordetella parapertussis, Bordetella bronchiseptica, Burkholderia cepacia, Citrobacter freundii, Chlamydia pneumoniae, Chlamydia trachomatis, Chlamydia psittaci, Enterobacter aerogenes, Enterobacter cloacae, Enterobacter sakazakii, Escherichia coli, Francisella tularensis, Haemophilus influenzae, Haemophilus aegypticus, Haemophilus ducreyi, Klebsiella edwardsii, Klebsiella pneumoniae, Legionella pneumophilia, Moraxella catarrhalis, Neisseria meningitidis, Neisseria gonorrhoeae, Proteus mirabilis, Proteus vulgaris, Pseudomonas aeruginosa, Rickettsia rickettsii, Rickettsia akari, Rickettsia conorrii, Rickettsia sibirica, Rickettsia australis, Rickettsia fells, Rickettsia japonica, Rickettsia africae, Rickettsia provvazekii, Rickettsia typhi, Salmonella enterica, Shigella boydii, Shigella dysenteriae, Shigella fiexneri, Shigella sonnei, Stenotrophornonas maitophilia, Vibrio cholerae, Vibrio parahaemolyticus, Vibrio vulnificus, Vibrio fluviaiis, Yersinia pestis, Yersina enterocolitica, and Yersina pseudotuberculosis,

In a further aspect, the Gram negative bacteria is a multi-drug resistant Gram negative bacteria strain (MDR-GNB). In a still further aspect, the multi-drug resistant Gram negative bacteria strain (MDR-GNB) is resistant to at least one anti-microbial agent selected from amikacin, tobramycin, cefepime, ceftazidime, imipenem, meropenem, piperacillin-tazobactam, ciprofloxacin, levofloxacin, tigecycline, and polymyxin B. In yet a further aspect, the multi-drug resistant Gram negative bacteria strain (MDR-GNB) is selected from Acinetobacter sp., Enterobacter sp., Klebsiella sp., and Pseuodomonas sp. In an even further aspect, the multi-drug resistant Gram negative bacteria strain (MDR-GNB) is selected from Acinetobacter baumannii, Enterobacter aerogenes, Klebsiella pneumoniae, and Pseudomonas aeruginosa. In a still further aspect, the multi-drug resistant Gram negative bacteria strain (MDR-GNB) is Enterobacter sp.

In a further aspect, the present disclosure provides methods of treating an infectious disease selected from atypical pneumonia, bacterial meningitis, bronchitis, cholera, dental infection, dermatitis, diarrhea, diphtheria, dysentery, ear infection, endocarditis,gastritis, gastroenteritis, genital infection, genitourinary infection, infection associated with an indwelling device, intestinal infection, leprosy, listeriosis, lung infection, nocosomial infection, ocular infection, oral infection, otitis, osteo-articular infection, osteomyelitis, pharyngitis, papules, pharyngitis, pneumonia, pneumonia conjunctivitis, pruritius, pustules, pyoderma, pyothorax, respiratory infection, salmonellosis, septicemia, sexually transmitted disease, sinusitis, skin infection, skin and soft tissue infection (“SSTI”), soft tissue infection, tetanus, tuberculosis, typhus, ulcer, urinary tract infection, and wound infection. In a still further aspect, the infectious disease is selected from endocardititis, osteomyelitis, skin and soft tissue infection (“SSTI”), and infection associated with an indwelling device. In yet a further aspect, the infectious disease is endocardititis, In an even further aspect, the infectious disease is osteomyelitis. In a still further aspect, the infectious disease is an SSTI. In yet a further aspect, the SSTI is a complicated SSTI (cSSTI). In an even further aspect, the infectious disease is associated with an indwelling device.

In a further aspect, the present disclosure provides methods of treating an infectious disease such in a human subject comprising administering a disclosed compound or a disclosed pharmaceutical composition, and further comprising administering to the human subject a therapeutically effective amount of a second active agent. In a still further aspect, the second active agent comprises at least one antibacterial agent. In yet a further aspect, the antibacterial agent comprises a compound selected from amoxicillin, ampicillin, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, cycloserin, dalbavancin, dalfopristin, demeclocycline, dicloxaciilin, dirithromycin, doxycycline, erythromycin, enrofloxacin, enoxacin, enviomycin, ertepenern, ethambutol, ethionmide, flucloxacillin, fosfomycin, furazolidone, gatifloxacin, gentamicin, imipenem, isoniazid, kanamycin, levofloxacin, linezolid, lomefloxacin, loracarbef, mafenide, moxifloxacin, meropenem, metronidazole, mezlocillin, minocycline, mupirocin, nafcillin, nalidixic acid, neomycin, netilmicin, nitrofurantoin, norfloxacin, ofloxacin, oritavancin, oxytetracycline, penicillin, piperacillin, platensimycin, polyrnixin B, pyrazinamide, quinupristin, retapamulin, rifabutin, rifampin, rifapentine, roxithromycin, sparfloxacin, spectinomycin, sulbactam, sulfacetamide, sulfamethizole, sulfarnethoxazole, teicoplanin, telithromycin, telavancin, temafloxacin, tetracycline, thioacetazone, thioridazine, ticarcillin, tinidazole, tobramycin, torezolid, tosufloxacin, trimethoprim, troleandomycin, trovafioxacin, and vancomycin, or combinations thereof.

In a further aspect, the present disclosure provides methods of treating an infectious disease such in a human subject comprising administering a disclosed compound or a disclosed pharmaceutical composition, and further comprising administering to the human subject a therapeutically effective amount of an anti-tuberculosis agent. In a still further aspect, the anti-tuberculosis agent is selected from amikacin, amoxicillin-clavulanic acid, bedaquiline, capreomycin, ciprofloxacin, clarithromycin, clofazimine, cycloserine, ethambutol, ethionamide, gatifloxacin, imipenem, isoniazid, kanamycin, levofloxacin, meropenem, moxifloxacin, ofloxacin, OPC-7683, para-aminosalicylic acid, pretomanid, pyrazinamide, rifampin, rifapentine, rifabutin, SQ109, streptomycin, sudoterb, terizidone, thiacetazone, viomycin, and combinations thereof. In a yet further aspect, the anti-tuberculosis agent is an aminoglycoside antibiotic, such as kanamycin A, amikacin, tobramycin, dibekacin, gentamicin, sisomicin, netilmicin, neomycin B, neomycin C, paromomycin and streptomycin. In an even further aspect, the anti-tuberculosis agent is a fluroguinolone, such as moxifloxacin, levofloxacin, sparfloxacin, nalidixic acid, ciprofloxacin, cinoxacin, oxolinic acid, piromidic acid, pipemidic acid, rosoxacin, enoxacin, fleroxacin, lornefloxacin, nadifloxacin, norfloxacin, ofloxacin, perfloxacin, rufloxacin, balofloxacin, grepafloxacin, pazufloxacin, temafloxacin, tosufloxacin, clinafloxacin, gatlifloxacin, sitafloxacin, prulifloxacin, delafloxacin, JNJ-Q2, nemofloxacin, danofloxacin, difloxacin, enrofloxacin, ibafloxacin, marbofloxacin, orbifloxacin, sarafloxacin and trovafloxacin. In a still further aspect, the anti-tuberculosis agent is a nitroimidazole antibiotic, such as metronidazole, tinidazole and nimorazole.

In a further aspect, the present disclosure provides methods of treating an infectious disease such in a human subject comprising administering a disclosed compound or a disclosed pharmaceutical composition, and further comprising administering to the human subject a therapeutically effective amount of an immunomodulatory agent. In a still further aspect, the immunomodulatory agent is a cytokine, an interleukin, a chemokine, or combinations thereof, In a yet further aspect, the immunomodulatory agent is selected from IL-2, IL-7 and IL-12, IFN-α, IFN-β, IFN-ε, IFN-κ, IFN-γ, IFN-γ 1b, CCL3, CCL26, CXCL7, and combinations thereof.

In a further aspect, the administering is co-administering of the disclosed compound and the antibacterial agent. In a still further aspect, the co-administration is administration in a substantially simultaneous manner of the disclosed compound and the antibacterial agent. In yet a further aspect, the co-administration is administration in a substantially sequential manner of the disclosed compound and the antibacterial agent.

In a further aspect, the administration in a substantially simultaneous manner comprises a single dose form containing a fixed ratio of the compound and the antibacterial agent. In a still further aspect, the single dose form is a capsule or a tablet. In yet a further aspect, the single dose form is an ampule for a single intravenous administration.

In various aspects, the disclosed compounds can have a mechanism of antimicrobial action and/or may bind to and/or inhibit one or more bacterial target molecules or macromolecular complexes containing a bacterial target molecule. Mechanisms of action may include inhibiting or interfering with a biological or biochemical pathway of the bacterium. Exemplary pathways include, but are not limited to, protein synthesis, cell wall synthesis, DNA replication, transcription, and cell division. It will be appreciated that biological and biochemical pathways are not mutually exclusive and that some biological or biochemical pathways may be considered to be subsets or sub-pathways of other biological or biochemical pathways. Mechanisms of action include, but are not limited to, inhibiting protein synthesis (e.g., by binding ribosomal RNA or proteins, blocking tRNA binding to the ribosome-mRNA complex, inhibiting peptidyl transferase), inhibiting or interfering with synthesis of a cell wall component (e.g., inhibition of peptidoglycan synthesis, disruption of peptidoglycan cross-linkage, disruption of movement of peptidoglycan precursors, disruption of mycotic acid or arabinoglycan synthesis), cell membrane disruption, inhibiting or interfering with nucleic acid synthesis of processing, acting as “antimetabolites” and either inhibiting an essential bacterial enzyme or competing with a substrate of an essential bacterial enzyme, inhibiting or interfering with cell division.

Molecules, or macromolecular complexes containing them, that may be targets for antibiotics include, but are not limited to, peptidoglycans, penicillin binding proteins, lipopolysaccharides, ribosomes or ribosomal subunits or RNA or protein components thereof (23S rRNA, 16S rRNA, proteins of the 30S or 50S subunit), DNA-dependent DNA polymerase, DNA-dependent RNA polymerase, microbial type I topoisomerase, microbial type II topoisomerase (e.g., topoisomerase IV or gyrase), enzymes involved in cell division such as FtsZ, etc.

In various aspects, the disclosed compounds inhibit bacterial protein synthesis. The bacterial species may be of any one or more types, e.g., gram-negative bacteria, gram-positive bacteria, atypical bacteria, and/or acid fast bacteria. Suitable organisms can include, but are not limited to members of the following genuses: Actinomyces, Staphylococcus, Streptococcus, Enterococcus, Erysipelothrix, Neisseria, Branhamella, Listeria, Bacillus, Corynbacterium, Erysipelothrix, Gardnerella, Mycobacterium, Nocardia, Enterobacteriaceae, Escherichia, Salmonella, Shigella, Yersinia, Enterobacter, Klebsiella, Citrobacter, Serratia, Providencia, Proteus, Morganella, Edwardsiella, Erwinia, Vibrio, Aeromonas, Helicobacter, Campyiobacter, Eikenella, Pasteurella, Pseudomonas, Burkhoideria, Stenotrophomonas, Acinetobacter, Ralstonia, Alcaligenes, Moraxella, Mycoplasma, Legionella, Francisella, Brucella, Haemophilus, Bordetella, Clostridium, Bacteroides, Porphyromonas, Prevotella, Fusobacterium, Borrelia, Chlamydia, Rickettsia, Ehrlichia, Bartonella, Trichomonas, and Treponema.

In various aspects of the disclosure the bacteria are species that are causative agents of disease in humans and/or animals: Examples include, but are not limited to, Acinetobacter baumannii, Aeromonas hydrophile, Bacillus anthracis, Bacillus anthracis sterne, Bacillus subtilis, Burkholderia cepacia, Escherichia coli, Enterobacter cloacae, Enterococcus faecalis, Francisella tularensis, Campylobacterjejuni, Haemophilus influenzae, Klebsiella pneumoniae, Klebsiella oxytoca, Legionella pneumophila, Pasteurella multocida, Proteus finerabeles, Proteus vulgaris, Mycobacterium tuberculosis, Morganella morganii, Helicobacter pylori, Neisseria meningitides, Neisseria gonorrhoeae, Chlamydia trachomatis, Pseudomonas aeruginosa, Salmonella enterica, Salmonella typhimurium, Staphylococcus aureus, Staphylococcus epidermidis, Streptococcus pneumoniae, Streptococcus pyogenes, Strenotrophornonas maltophilia, Streptococcus agalactiae, and Yersinia pestis.

In a further aspect, disclosed are methods for inhibiting protein synthesis in at least one bacterial cell, comprising the step of contacting the bacterial cell with an effective amount of at least one disclosed compound, or a pharmaceutically acceptable salt thereof. That is, the disclosed compounds inhibit bacterial protein synthesis. The inhibition of bacterial protein synthesis can be demonstrated by methodology known in the art. For example, inhibition of bacterial protein synthesis can be determined by measurement of bacterial growth in response to a disclosed compound or in a cell-free ribosomal inhibition assay such as that described herein below. In some aspects, the disclosed compound is a prodrug that needs a prodrug moiety removed prior to assessing the effect of the disclosed compound in an assay.

### F. Manufacture of a Medicament

In various aspects, the present disclosure pertains to uses of a disclosed compound, or a pharmaceutically acceptable salt thereof, in the manufacture of a medicament with a pharmaceutically acceptable carrier or diluent for the treatment of a disorder associated with a microbial infection in a mammal. In a further aspect, the present disclosure pertains to methods for the manufacture of a medicament to inhibit protein synthesis in a microbe infecting a mammal comprising combining at least one disclosed compound, or a pharmaceutically acceptable salt thereof in the manufacture of a medicament with a pharmaceutically acceptable carrier or diluent.

In one aspect, the disclosure relates to a medicament comprising one or more disclosed compounds; or a pharmaceutically acceptable salt, hydrate, solvate, or polymorph thereof, in a further aspect, the one or more compounds are a product of a disclosed method of making,

In various aspect, the disclosure relates methods for the manufacture of a medicament for inhibition of bacterial protein synthesis (e.g., treatment of one or more bacterial infections) in mammals (e.g., humans) comprising combining one or more disclosed compounds, products, or compositions or a pharmaceutically acceptable salt, solvate, hydrate, or polymorph thereof, with a pharmaceutically acceptable carrier, It is understood that the disclosed methods can be performed 1.ivith the disclosed compounds, products, and pharmaceutical compositions. It is also understood that the disclosed methods can be employed in connection with the disclosed methods of using.

### G. Uses of Disclosed Compounds

In various aspects, the present disclosure relates to uses of the disclosed compounds and products. in one aspect, the disclosure relates to use of at least one disclosed compound; or a pharmaceutically acceptable salt, hydrate, solvate, or polymorph thereof. In a further aspect, the compound used is a product of a disclosed method of making.

In one aspect, the disclosure relates to the use of a compound in the manufacture of a medicament for the treatment of infectious diseases, wherein the compound is a disclosed compound; or a pharmaceutically acceptable salt, hydrate, solvate, or polymorph thereof.

In a further aspect, the disclosure relates to the use of a compound in the manufacture of a medicament for the treatment of infectious diseases, wherein the compound is a product of a disclosed method of making; or a pharmaceutically acceptable salt, hydrate, solvate, or polymorph thereof.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to the treatment of an infectious disases in a vertebrate animal. In a further aspect, the use relates to the treatment of an infectious disease in a human subject.

In a further aspect, the use is the treatment of an infectious disease. In a still further aspect, the infectious disease is associated with a Gram positive bacterial infection. In yet a further aspect, the infectious disease is associated with a Gram negative bacterial infection.

It is understood that the disclosed uses can be employed in connection with the disclosed compounds, methods, compositions, and kits. In a further aspect, the disclosure relates to the use of a disclosed compound or a disclosed product in the manufacture of a medicament for the treatment of a bacterial infection in a mammal.

In a further aspect, the disclosure relates to the use of a disclosed compound or a disclosed product in the manufacture of a medicament for the treatment of an infectious disease selected from atypical pneumonia, bacterial meningitis, bronchitis, cholera, dental infection, dermatitis, diarrhea, diphtheria, dysentery, ear infection, endocarditimastritis, gastroenteritis, genital infection, genitourinary infection, infection associated with an indwelling device, intestinal infection, leprosy, listeriosis, lung infection, nocosomial infection, ocular infection, oral infection, otitis, osteo-articular infection, osteomyelitis, pharyngitis, papules, pharyngitis, pneumonia, pneumonia conjunctivitis, pruritius, pustules, pyoderma, pyothorax, respiratory infection, salmonellosis, septicemia, sexually transmitted disease, sinusitis, skin infection, skin and soft tissue infection (“SSTI”), soft tissue infection, tetanus, tuberculosis, typhus, ulcer, urinary tract infection, and wound infection.

In a further aspect, the disclosure relates to the use of a disclosed compound or a disclosed product in the manufacture of a medicament for the treatment of tuberculosis.

In a further aspect, the disclosure relates to the use of a disclosed compound or a disclosed product in the manufacture of a medicament for the treatment of a bacterial infection associated with infection by with a bacterial species selected from Bacillus sp. Clostridium sp., Corynebacterium sp, Enterococcus sp., Mycoplasma sp., Staphylococcus sp., and Streptococcus sp.

In a further aspect, the disclosure relates to the use of a disclosed compound or a disclosed product in the manufacture of a medicament for the treatment of a bacterial infection associated with infection by with a bacterial species selected from Bacillus anthracis, Bacillus cereus, Bacillus subtilis, Clostridium difficile, Clostridium tetani, Clostridium botulinum, Clostridium perfringens, Corynebacterium diphtheria, Enterococcus faecalis, Enterococcus faecium, Listeria monocytogenes, Listeria ivanovii, Micrococcus luteus, Mycoplasma genitalium, Mycoplasma pneumoniae, Staphylococcus aureus, Staphylococcus epidermidis, Staphylococcus saprophyticus, Staphylococcus hyicus, Staphylococcus intermedius, Streptococcus pneumoniae, and Streptococcus pyogenes.

In a further aspect, the disclosure relates to the use of a disclosed compound or a disclosed product in the manufacture of a medicament for the treatment of a bacterial infection associated with infection by with a bacterial species selected from Acinetobacter sp., Aeromonas sp., Burkholderia sp., Bordetella sp., Citrobacter sp., Chlamydia sp., Enterobacter sp., Escherichia sp., Francisella sp., Haemophilus sp., Klebsiella sp., Legionella sp., Moraxella sp., Neisseria sp., Proteus sp., Pseudomonas sp., Rickettsia sp., Salmonella sp., Shigella sp., Stenotrophomonas sp., Vibrio sp., and Yersinia sp.

In a further aspect, the disclosure relates to the use of a disclosed compound or a disclosed product in the manufacture of a medicament for the treatment of a bacterial infection associated with infection by with a bacterial species selected from Acinetobacter baumannii. Aeromonas hydrophile, Bordetella pertussis, Bordetella parapertussis, Bordetella bronchiseptica, Burkholderia cepacia, Citrobacter freundii, Chlamydia pneumoniae, Chlamydia trachomatis, Chlamydia psittaci, Enterobacter aerogenes, Enterobacter cloacae, Enterobacter sakazakii, Escherichia coil, Francisella tularensis, Haemophilus influenzae, Haemophilus aegypticus, Haemophilus ducreyi, Klebsiella edwardsii, Klebsiella pneumoniae, Legionella pneumophilia, Moraxella catarrhalis, Neisseria meningitidis, Neisseria gonorrhoeae, Proteus mirabilis, Proteus vulgaris, Pseudomonas aeruginosa. Rickettsia rickettsii, Rickettsia akari, Rickettsia conorrii, Rickettsia sibirica, Rickettsia australis, Rickettsia fells, Rickettsia japonica, Rickettsia africae, Rickettsia prowazekii, Rickettsia typhi, Salmonella enterica, Shigella boydii, Shigella dysenteriae, Shigella flexneri, Shigella sonnei, Stenotrophomonas maltophilia, Vibrio cholerae, Vibrio parahaemolyticus, Vibrio vulnificus, Vibrio fluvialis, Yersinia pestis, Yersina enterocolitica, and Yersina pseudotuberculosis.

### H. Kits

In various aspects, disclosed are kits comprising at least one disclosed compound, or a pharmaceutically acceptable salt thereof; or a disclosed pharmaceutical composition; and one or more of: (a) at least one agent known to microbial ribosomal activity; (b) at least one agent known to have antimicrobial activity; (c) at least one agent known to treat an infectious disease; (d) instructions for treating an infectious disease; (e) instructions for administering the compound in connection with treating a microbial infection; or (f) instructions for administering the compound with at least one agent known to treat an infectious disease.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present disclosure also features such kits further containing instructions for use.

In a further aspect, the present disclosure also provides a pharmaceutical pack or kit comprising one or more containers filled with one or more of the ingredients of the pharmaceutical compositions of the disclosure. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### I. Research Tools

The disclosed compounds and pharmaceutical compositions have activity as inhibitors of bacterial protein synthesis. As such, the disclosed compounds are also useful as research tools, Accordingly, one aspect of the present disclosure relates to a method of using a compound of the disclosure as a research tool, the method comprising conducting a biological assay using a disclosed compound. Disclosed compounds can also be used to evaluate new chemical compounds. Thus another aspect of the disclosure relates to a method of evaluating a test compound in a biological assay, comprising: (a) conducting a biological assay with a test compound to provide a first assay value; (b) conducting the biological assay with a compound of the disclosure to provide a second assay value; wherein step (a) is conducted either before, after or concurrently with step (b); and (c) comparing the first assay value from step (a) with the second assay value from step (b). Exemplary biological assays include a assay that can be conducted in vitro or in a cell culture system as disclosed herein. Still another aspect of the disclosure relates to a method of studying a biological system, e.g., a model animal for a clinical condition, the method comprising: (a) contacting the biological system or sample with a compound of the disclosure; and (b) determining the effects caused by the compound on the biological system or sample. Exemplary in vivo model systems are disclosed herein.

### J. Aspects

The following listing of exemplary aspects supports and is supported by the disclosure provided herein.

Aspect 1. A compound having a structure represented by a formula:

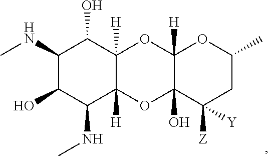

wherein Y is hydrogen or hydroxyl; wherein Z is —CH₂NH—(C1-C3 alkanediyl)Ar¹ or —NH—(C═O)—(C1-C3 alkanediyl)-Ar¹; wherein Ar¹ is aryl or heteroaryl substituted with: (a) a R²⁰ group, wherein R²⁰ is selected from —(C1-C3)-alkanediyl-OP(O)(OR²¹) (OR²²), —OP(O)(OR²¹)(OR²²), —C1-C3)-alkanediyl-OSO₂OR²¹, —OSO₂OR²¹, —(C1-C3)-alkanediyl-OSO₂R²¹, —OSO₂R²¹, —(C1-C3)-alkanediyl-OSO₂NR²¹R²², —OSO₂NR²¹R²², wherein each of R²¹ and R²² is independently selected from hydrogen and C1-C3 alkyl; and (b) Cto 2 groups independently selected from halo, cyano, hydroxyl, —NH₂, C1-C3 alkyl, C1-C3 haloalkyl, C1-C3 alkoxy, and C1-C3 haloalkoxy; or a pharmaceutically acceptable salt thereof.

Aspect 2. The compound according to Aspect 1, wherein Y is hydrogen.

Aspect 3. The compound according to Aspect 1, wherein Y is hydroxyl.

Aspect 4. The compound according to any one of 1-Aspect 3, wherein Z is —CH₂—NH—(C1-C3 alkanediyl)-Ar¹.

Aspect 5. The compound according to Aspect 4, wherein Z is —CH₂—NH—(CH₂)m-Ar¹; and wherein m is an integer selected from 0, 1, 2, and 3.

Aspect 6. The compound according to Aspect 5, wherein m is 1 or 2.

Aspect 7. The compound according to Aspect 5, wherein m is 1.

Aspect 8. The compound according to Aspect 5, wherein m is 2.

Aspect 9. The compound according to Aspect 1, wherein Z is —NH—(C═O)—(C1-C3 alkanediyl)-Ar¹.

Aspect 10. The compound according to Aspect 9, wherein Z is —NH—(C═O)—(CH₂)q-Ar¹; and wherein q is an integer selected from 0, 1, 2, and 3.

Aspect 11. The compound according to Aspect 10, wherein q is 1 or 2,

Aspect 12. The compound according to Aspect 10, wherein q is 1.

Aspect 13. The compound according to Aspect 10, wherein q is 2.

Aspect 14. The compound according to any one of Aspect 1-Aspect 13, wherein Ar¹ is a structure represented by a formula:

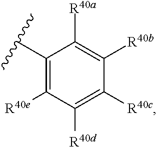

wherein 1 of R^{40a}, R^{40b}, R^{40c}, R^{40d} and R^{40e} is R²⁰; wherein 2, 3, or 4 of R^{4a}, R^{40b}, R^{40c}, R^{40d}, and R^{40e} are independently hydrogen; and, wherein 0, 1, or 2 of R^{40a}, R^{40b}, R^{40c}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

Aspect 15. The compound of Aspect 14, wherein 1 of R^{40a}, R^{40b}, R^{40c}, R^{40d}, and R^{40e} is R²⁰; wherein 2, 3, or 4 of R^{40a}, R^{40b}, R^{40c}, R^{40d}, and R^{40e} are independently hydrogen; and, wherein 0, 1, or 2 of R^{40a}, R^{40b}, R^{40d}, and R^{40e} are independently selected from halo.

Aspect 16. The compound of Aspect 15, wherein Ar¹ is a structure represented by a formula:

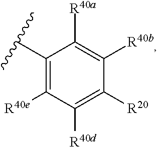

wherein 0, 1, or 2 of R^{40a}, R^{40b}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

Aspect 17. The compound of Aspect 16, wherein each of R^{40a}, R^{40b}, R^{40d}, and R^{40e} is hydrogen.

Aspect 18. The compound of Aspect 16, wherein R^{40a} is hydrogen or halo; and wherein each of R^{40b}, R^{40d}, and R^{40e} is hydrogen.

Aspect 19. The compound of Aspect 16, wherein R^{40a} is hydrogen or fluoro; and wherein each of R^{40b}, R^{40d}, and R^{40e} is hydrogen.

Aspect 20. The compound of any one of Aspect 14-Aspect 19 vherein R²⁰ is —(C1-C3)-alkanediyl-OP(O)(OR²¹)(OR²²) or —OP(O)(OR²¹)(OR²′),

Aspect 21. The compound of Aspect 20, wherein R²⁰ is —(C1-C3)-alkanediyl-OP(O)(OR²¹)(OR²²).

Aspect 22. The compound of Aspect 21, wherein R²⁰ is —(CH₂)—OP(O)(OR²¹)(OR²²).

Aspect 23. The compound of Aspect 21, wherein R²⁰ is —CH(CH3))—OP(O)(OR²¹)(OR²²).

Aspect 24. The compound of Aspect 20, wherein R²⁰ is —OP(O)(OR²¹)(OR²²).

Aspect 25. The compound of any one of Aspect 20-Aspect 24, wherein each of R²¹ and R²² is independently selected from hydrogen, methyl, and ethyl.

Aspect 26. The compound of any one of Aspect 20-Aspect 24, wherein each of R²¹ and R²² is hydrogen.

Aspect 27. The compound of any one of Aspect 14-Aspect 19, wherein R²⁰ is —(C1-C3)-alkanediyl-OSO₂OR²¹ or —OSO₂OR²¹.

Aspect 28. The compound of Aspect 27, wherein R²⁰ is —(C1-C3)-alkanediyl-OSO₂OR²¹.

Aspect 29. The compound of Aspect 28, wherein R²⁰ is —(CH₂)—OSO₂OR²¹.

Aspect 30. The compound of Aspect 28, wherein R²⁰ is —(CH(CH3))-OSO₂OR²¹.

Aspect 31. The compound of Aspect 27, wherein R²⁰ is —OSO₂OR²¹.

Aspect 32. The compound of any one of Aspect 27-Aspect 31, wherein R²¹ is selected from hydrogen, methyl, and ethyl.

Aspect 33. The compound of any one of Aspect 27-Aspect 31, wherein R²¹ is hydrogen.

Aspect 34. The compound according to any one of 1-Aspect 13, wherein Ar¹ is a structure represented by a formula:

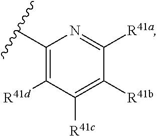

wherein 1 of R^{41a}, R^{41b}, R^{41c}, and R^{41d} is R²⁰; wherein 1, 2, or 3 of R^{41a}, R^{41b}, R^{41c}, and R^{41d} are independently hydrogen; and, wherein 0, 1, or 2 of R^{41a}, R^{41b}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

Aspect 35. The compound of Aspect 14, wherein 1 of R^{41a}, R^{41b}, R^{41c}, and R^{41d} is R²⁰;

wherein 1, 2, or 3 of R^{41a}, R^{41b}, R^{41c}, and R^{41d} are independently hydrogen: and, wherein 0, 1, or 2 of R^{41a}, R^{41b}, R^{41c}, and R^{41d} are independently selected from halo.

Aspect 36. The compound according to Aspect 34, wherein Ari is a structure represented by a formula:

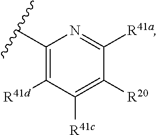

wherein 0, 1, or 2 of R^{41a,} R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

Aspect 37. The compound of Aspect 36, wherein each of R^{41a}, R⁴¹c, and R^{41d} is hydrogen.

Aspect 38. The compound of Aspect 36, wherein R^{40a} is hydrogen or halo; and wherein each of R^{41a}, and R^{41c} is hydrogen.

Aspect 39. The compound of Aspect 36, wherein R^{40a} is hydrogen or fluoro; and wherein each of R^{41a}, R^{41c}, and R^{41d} is hydrogen.

Aspect 40. The compound of any one of Aspect 34-Aspect 39, wherein R²⁰ is —(C1-C3)-alkanediyl-OP(O)(OR²¹)(OR²²) or —OP(O)(OR²¹)(OR²²).

Aspect 41. The compound of Aspect 40, wherein R²⁰ is —(C1-C3)-alkanediyl-OP(O)(OR²¹)(OR²²).

Aspect 42. The compound of Aspect 41, wherein R²⁰ is —(CH₂)—OP(O)(OR²¹)(OR²²).

Aspect 43. The compound of Aspect 41, wherein R²⁰ is —(CH(CH3))—OP(O)(OR²¹)(OR²²).

Aspect 44. The compound of Aspect 40, wherein R²⁰ is CP(O)(OR²¹)(OR²²).

Aspect 45. The compound of any one of Aspect 40-Aspect 44, wherein each of R²¹ and R²² is independently selected from hydrogen, methyl, and ethyl.

Aspect 46. The compound of any one of Aspect 40-Aspect 44, wherein each of R²¹ and R²² is hydrogen.

Aspect 47. The compound of any one of Aspect 34-Aspect 39, wherein R²⁰ is —(C1-C3)-alkanediyl-OSO₂OR²¹ or —OSO₂OR²¹.

Aspect 48. The compound of Aspect 47, wherein R²⁰ is —(C1-C3)-alkanediyl-OSO₂OR²¹,

Aspect 49. The compound of Aspect 48, wherein R²⁰ is —(CH₂)—OSO₂OR²¹.

Aspect 50. The compound of Aspect 48, wherein R²⁰ is —(CH(CH3))-OSO₂OR²¹.

Aspect 51. The compound of Aspect 47, wherein R²⁰ is —OSO₂OR²¹.

Aspect 52. The compound of any one of Aspect 47-Aspect 51, wherein R²¹ is selected from hydrogen, methyl, and ethyl.

Aspect 53. The compound of any one of Aspect 47-Aspect 51, wherein R²¹ is hydrogen.

Aspect 54. The compound according to any one of 1-Aspect 13, wherein Ar¹ is a structure represented by a formula;

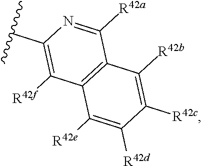

wherein 1 of R^{42a}, R^{42b}, R^{42c}, R^{42d}, R^{42e}, and R^{42f} is R²⁰; wherein 3, 4, or 5 of R^{42a}, R^{42b}, R^{42c}, R^{42d}, R^{42e}, and R^{42f} are independently hydrogen; and, wherein 0, 1, or 2 of R^{42a}, R^{42b}, R^{42c}, R^{42d}, R^{42e}, and R^{42f} are independently selected from halo, cyano, hydroxyl, —NH₂, C1-C3 alkyl, C1-C3 haloalkyl, C1-C3 alkoxy, and C1-C3 haloalkoxy.

Aspect 55. The compound of Aspect 54, wherein 1 of R^{42a}, R^{42b}, R⁴²d, R^{42e} and R^{42f}; wherein 3, 4, or 5 of R^{42a}, R^{42b}, R^{42c}, R42d, R^{42e}, and R^{42f} are independently hydrogen; and, wherein 0, 1, or 2 of R^{42a}, R^{42b}, R^{42c}, R42d, R^{42e}, and R^{42f} are independently selected from halo.

Aspect 56. The compound according to Aspect 54, wherein Ar¹ is a structure represented by a formula:

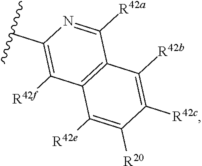

wherein 0, 1, or 2 of _{R}^{42a}, R^{42b}, R^{42c}, R^{42e} and R^{42f} are independently selected from halo, cyano, hydroxyl, —NH₂, C1-C3 alkyl, C1-C3 haloalkyl, C1-C3 alkoxy, and C1-C3 haloalkoxy.

Aspect 57. The compound of Aspect 56, wherein each of R^{42a}, R^{42b}, R^{42c}, R^{42e}, and R^{42f} is hydrogen.

Aspect 58. The compound of Aspect 56, wherein one of R^{42a}, R^{42b}, R^{42c}, R^{42e}, and R^{42f} is halo; and wherein four of R^{42a}, R^{42b}, R^{42c}, R^{42e}, and R^{42f} are hydrogen.

Aspect 59. The compound of Aspect 56, wherein one of R^{42a}, R^{42b}, R^{42c}, R^{42e}, and R^{42f} is fluoro; and wherein four of R^{42a}, R^{42b}, R^{42c}, R^{42e}, and R^{42f} are hydrogen.

Aspect 60. The compound of any one of Aspect 54-Aspect 59, wherein R²⁰ is —(C1-C3)-alkanediyl-OP(O)(OR²¹)(OR²²) or —OP(O)(OR²¹)(OR²²).

Aspect 61. The compound of Aspect 60, wherein R²⁰ is —(C1-C3)-alkanediyl-OP(O)(OR²¹)(OR²²).

Aspect 62. The compound of Aspect 61, wherein R²⁰ is —(CH₂)—OP(O)(OR²¹)(OR²²).

Aspect 63. The compound of Aspect 61, wherein R²⁰ is —(CH(CH3))—OP(O)(OR²¹)(OR²²).

Aspect 64. The compound of Aspect 60, wherein R²⁰ is —OP(O)(OR²¹)(OR²²).

Aspect 65. The compound of any one of Aspect 60-Aspect 64, wherein each of R²¹ and R²² is independently selected from hydrogen, methyl, and ethyl

Aspect 66. The compound of any one of Aspect 60-Aspect 64, wherein each of R²¹ and R²² is hydrogen.

Aspect 67. The compound of any one of Aspect 54-Aspect 59, wherein R²⁰ is —(C1-C3)-alkanediyl-OSO₂OR²¹ or —OSO₂OR²¹.

Aspect 68. The compound of Aspect 67, wherein R²⁰ is —(C1-C3)-alkanediyl-OSO₂OR²¹.

Aspect 69. The compound of Aspect 68, wherein R²⁰ is —(CH₂)—OSO₂OR²¹.

Aspect 70. The compound of Aspect 68, wherein R²⁰ is —(CH(CH3))-OSO₂OR²¹.

Aspect 71. The compound of Aspect 67, wherein R²⁰ is —OSO₂OR²¹.

Aspect 72. The compound of any one of Aspect 67-Aspect 71, wherein R²¹ is selected from hydrogen, methyl, and ethyl.

Aspect 73. The compound of any one of Aspect 67-Aspect 71, wherein R²¹ is hydrogen.

Aspect 74. The compound according to any one of 1-Aspect 13, wherein Ar¹ is a structure represented by a formula:

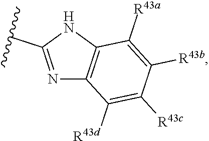

wherein 1 of R^{43a}, R^{43b}, R^{43c}, and R^{43d} is R²⁰; wherein 1, 2, or 3 of R^{43a}, R^{43b}, R^{43c}, and R^{43d} are independently hydrogen; and, wherein 0, 1, or 2 of R^{43a}, R^{43b}, R^{43c}, and R^{43d} are independently selected from halo, cyano, hydroxyl, —NH₂, C1-C3 alkyl, C1-C3 haloalkyl, C1-C3 alkoxy, and C1-C3 haloalkoxy.

Aspect 75. The compound of Aspect 74, wherein 1 of R^{43a}, R^{43b}, R^{43c}, and R^{43d} is R²⁰; wherein 1, 2, or 3 of R^{43a}, R^{43b}, and R^{43d} are independently hydrogen; and, wherein 0, 1, or 2 of R^{43a}, R^{43b}, R^{43c}, and R^{43d} are independently selected from halo.

Aspect 76. The compound according to Aspect 74, wherein Ar¹ is a structure represented by a formula:

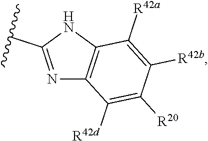

wherein 0, 1, or 2 of R^{43a}, R^{43b}, and R^{43d} are independently selected from halo, cyano, hydroxyl, —NH₂, C1-C3 alkyl. C1-C3 haloalkyl. C1-C3 alkoxy, and C1-C3 haloalkoxy.

Aspect 77, The compound of Aspect 76, wherein each of R^{40a}, R^{40d}, and R^{40e} is hydrogen.

Aspect 78. The compound of Aspect 76, wherein one of R^{40a}, R^{40d}, and R^{40e} is halo; and wherein two of R^{40a}, R^{40d}, and R^{40e} are hydrogen.

Aspect 79. The compound of Aspect 76, wherein one of R^{40a}, R^{40d}, and R^{40e} is fluoro; and wherein two of R^{40a}, R^{40d}, and R^{40e} are hydrogen.

Aspect 80. The compound of any one of Aspect 74-Aspect 79, wherein R²⁰ is —(C1-C3)-alkanediyl-OP(O)(OR²¹)(OR²²) or —OP(O)(OR²¹)(OR²²).

Aspect 81. The compound of Aspect 80, wherein R²⁰ is —(C1-C3)-alkanediyl-OP(O)(OR²¹)(OR²²).

Aspect 82. The compound of Aspect 81, wherein R²⁰ is —(CH₂)—OP(O)(OR²¹)(OR²²).

Aspect 83. The compound of Aspect 81, wherein R²⁰ is —(CH(CH3))-OP(O)(OR²¹)(OR²²).

Aspect 84. The compound of Aspect 80, wherein R²⁰ is OP(O)(OR²¹)(OR²²).

Aspect 85. The compound of any one of Aspect 80-Aspect 84, wherein each of R²¹ and R²² is independently selected from hydrogen, methyl, and ethyl.

Aspect 86. The compound of any one of Aspect 80-Aspect 84, wherein each of R²¹ and R²² is hydrogen.

Aspect 87. The compound of any one of Aspect 74-Aspect 79, wherein R²⁰ is —(C1-C3)-alkanediyl-OSO₂OR²¹ or —OSO₂OR²¹.

Aspect 88. The compound of Aspect 87, wherein R²⁰ is —(C1-C3)-alkanediyl-OSO₂OR²¹.

Aspect 89. The compound of Aspect 88, wherein R²⁰ is —(CH₂)—OSO₂OR²¹.

Aspect 90. The compound of Aspect 88, wherein R²⁰ is —(CH(CH3))-OSO₂OR²¹.

Aspect 91. The compound of Aspect 87, wherein R²⁰ is —OSO₂OR²¹.

Aspect 92. The compound of any one of Aspect 87-Aspect 91, wherein R²¹ is selected from hydrogen, methyl, and ethyl.

Aspect 93. The compound of any one of Aspect 87-Aspect 91, wherein R²¹ is hydrogen.

Aspect 94. The compound according to any one of Aspect 1-Aspect 13, wherein Ar¹ is a structure represented by a formula:

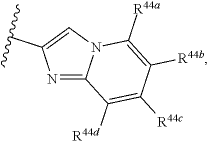

wherein 1 of R^{44a}, R^{44b}, R^{44c}, and R^{44d} is R²⁰; wherein 1, 2, or 3 of R^{44a}, R^{44b}, R^{44c}, and R^{44d} are independently hydrogen; and, wherein 0, 1, or 2 of R^{44a}, R^{44b}, R^{44c}, and R^{44d} are independently selected from halo, cyano, hydroxyl, —NH₂, C1-C3 alkyl, C1-C3 haloalkyl, C1-C3 alkoxy, and C1-C3 haloalkoxy.

Aspect 95. The compound of Aspect 94, wherein 1 of R^{44a}, R^{44b}, R^{44c}, and R^{44d} is R²⁰; wherein 1, 2, or 3 of R^{44a}, R^{44b}, R^{44c}, and R^{44d} are independently hydrogen; and wherein 0, 1, or 2 of R^{44a}, R^{44b}, R^{44c}, and R^{44d} are independently selected from halo.

Aspect 96. The compound according to Aspect 94, wherein Art is a structure represented by a formula:

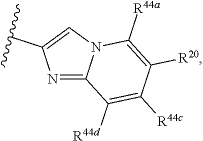

wherein 0, 1, or 2 of R^{44a}, R^{44c}, and R^{44d} are independently selected from halo, cyano, hydroxyl, —NH₂, C1-C3 alkyl, C1-C3 haloalkyl, C1-C3 alkoxy, and C1-C3 haloalkoxy.

Aspect 97, The compound of Aspect 96, wherein each of R^{44a}, R^{44c}, and R^{44d} is hydrogen.

Aspect 98. The compound of Aspect 96; wherein one of R^{44a}; R^{44c}, and R^{44d} is halo; and wherein two of R^{44a}, R^{44c}, and R^{44d} are hydrogen.

Aspect 99. The compound of Aspect 96, wherein one of R^{44a}, R^{44c}, and R^{44d} is fluoro; and wherein two of R^{44a}, R^{44c}, and R⁴⁴d are hydrogen.

Aspect 100. The compound according to Aspect 94, wherein Ar¹ is a structure represented by a formula;

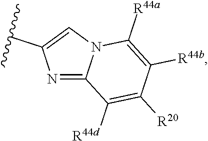

wherein 0, 1, or 2 of R^{44a}, R^{44b}, and R^{44d} are independently selected from halo, cyano, hydroxyl, —NH₂, C1-C3 alkyl, C1-C3 haloalkyl, C1-C3 alkoxy, and C1-C3 haloalkoxy.

Aspect 101. The compound of Aspect 100; wherein each of R^{44a}, R^{44b}, and R^{44d} is hydrogen.

Aspect 102. The compound of Aspect 100, wherein one of R^{44a}, R^{44b}; and R^{44d} is halo; and wherein two of R^{44a}, R^{44b}, and R^{44d} are hydrogen.

Aspect 103, The compound of Aspect 100, wherein one of R^{44a}, R^{44b}, and R^{44d} is fluoro; and wherein two of R^{44a}, R^{44b}, and R^{44d} 5are hydrogen.

Aspect 104. The compound of any one of Aspect 94-Aspect 103, wherein R²⁰ is —(C1-C3)-alkanediyl-OP(O)(OR²¹)(OR²²) or —OP(O)(OR²¹)(OR²²),

Aspect 105. The compound of Aspect 104, wherein R²⁰ is —(C1-C3)-alkanediyl-OP(O)(OR²¹)(OR²²).

Aspect 106. The compound of Aspect 105, wherein R²⁰ is —(CH₂)—OP(O)(OR²¹)(OR²²).

Aspect 107. The compound of Aspect 105, wherein R²⁰ is —(CH(CH3))—OP(O)(OR²¹)(OR²²).

Aspect 108. The compound of Aspect 104, wherein R²⁰ is —OP(O)(OR²¹)(OR²²).

Aspect 109. The compound of any one of Aspect 104-Aspect 108, wherein each of R²¹ and R²² is independently selected from hydrogen, methyl, and ethyl.

Aspect 110. The compound of any one of Aspect 104-Aspect 108, wherein each of R²¹ and R²² is hydrogen.

Aspect 111. The compound of any one of Aspect 94-Aspect 103, wherein R²⁰ is —(C1 C3)-alkanediyl-OSO₂OR²¹ or —OSO₂OR²¹.

Aspect 112. The compound of Aspect 111, wherein R²⁰ is —(C1-C3)-alkanediyl-OSO₂OR²¹.

Aspect 113. The compound of Aspect 112, wherein R²⁰ is —(C1-1₂)-OSO₂OR²¹.

Aspect 114. The compound of Aspect 112, wherein R²⁰ is —(CH(CH3))-OSO₂OR²¹.

Aspect 115. The compound of Aspect 111, wherein R²⁰ is —OSO₂OR²¹.

Aspect 116. The compound of any one of Aspect 111-Aspect 115, wherein R²¹ is selected from hydrogen, methyl, and ethyl.

Aspect 117. The compound of any one of Aspect 111-Aspect 115, wherein R²¹ is hydrogen.

Aspect 118. The compound of Aspect 1, present as:

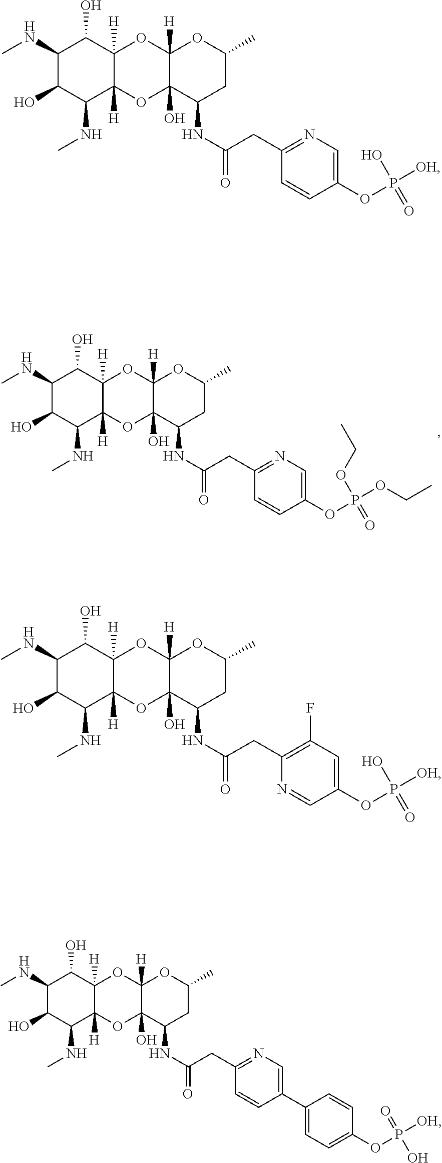

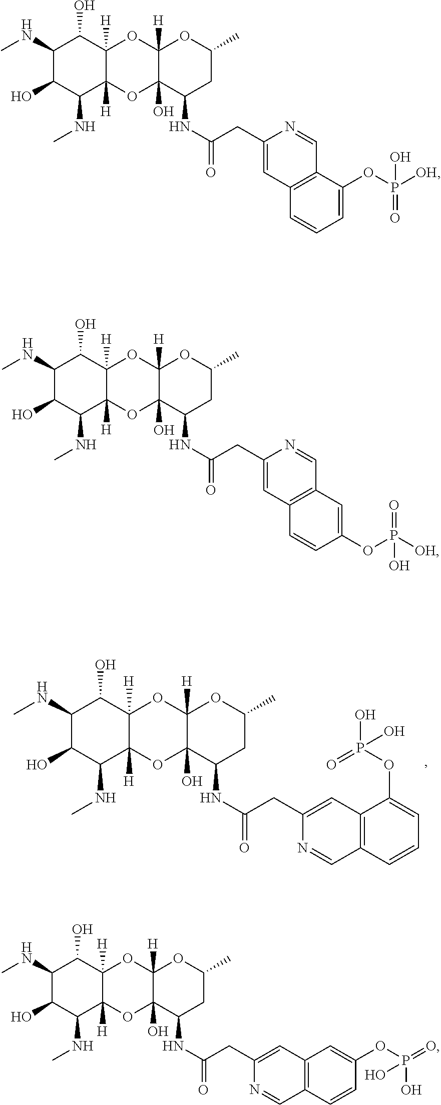 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1cc2cccc(OP(=O)(O)O)c2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2ccc(OP(=O)(O)O)cc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2c(OP(=O)(O)O)cccc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2cc(OP(=O)(O)O)ccc2cn1]

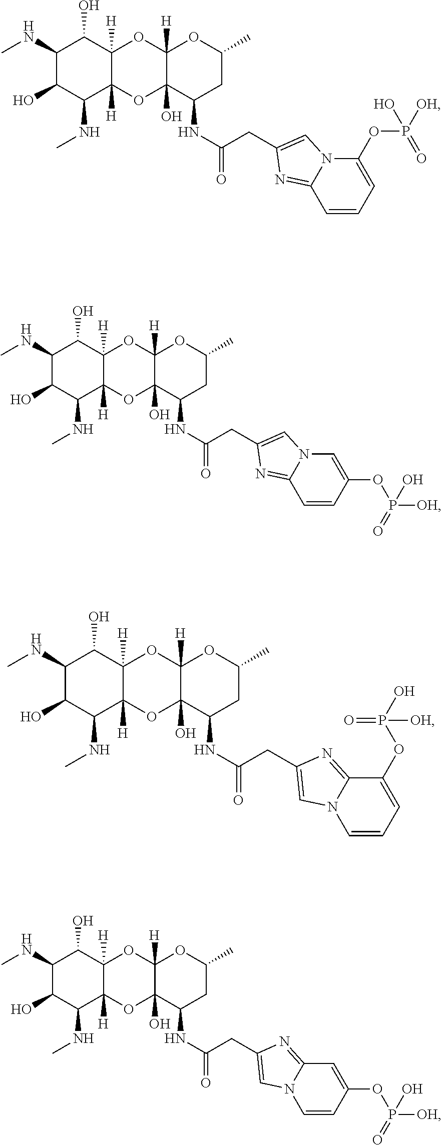 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1cn2c(OP(=O)(O)O)cccc2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2cc(OP(=O)(O)O)ccc2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2cccc(OP(=O)(O)O)c2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2ccc(OP(=O)(O)O)cc2n1]

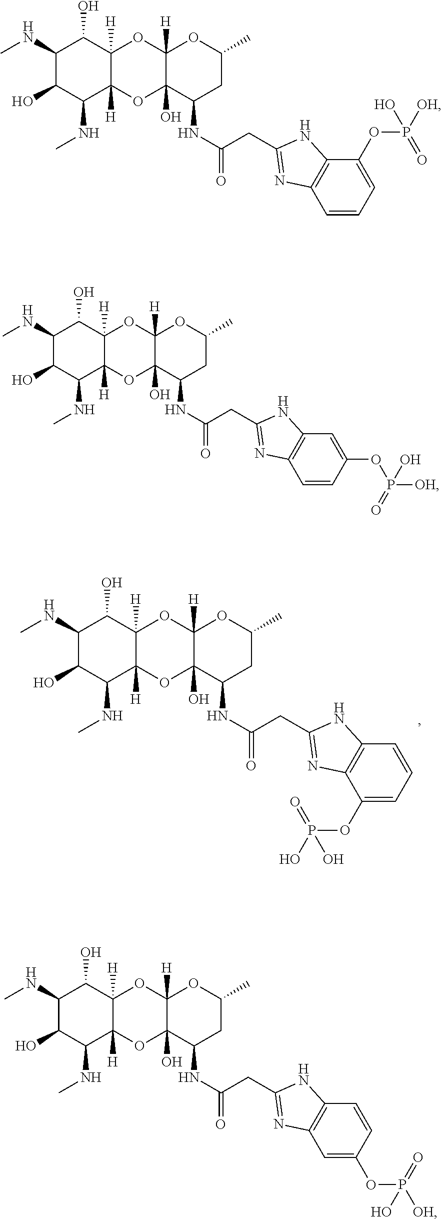 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1nc2cccc(OP(=O)(O)O)c2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2ccc(OP(=O)(O)O)cc2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2c(OP(=O)(O)O)cccc2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2cc(OP(=O)(O)O)ccc2[nH]1]

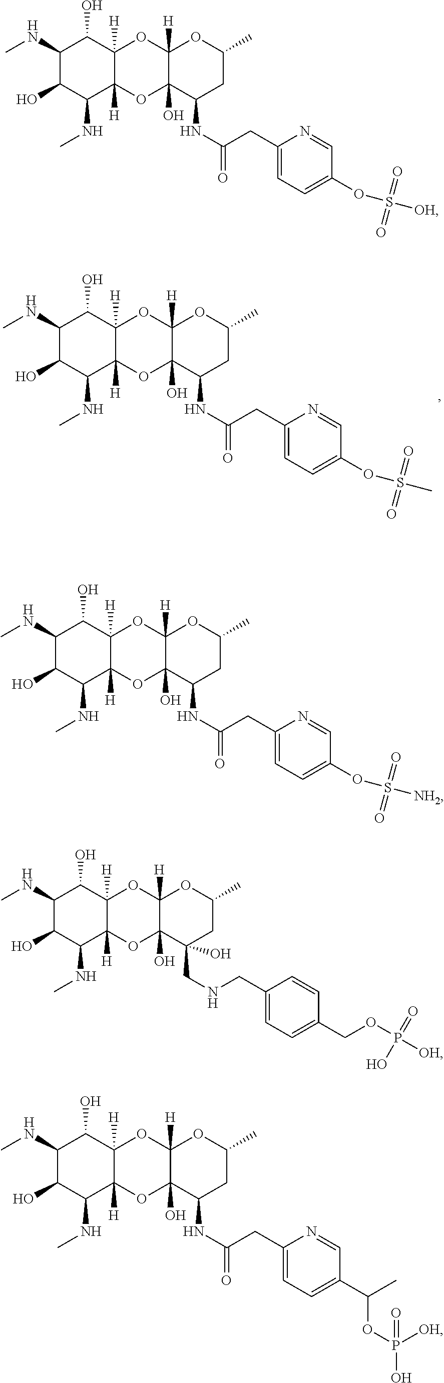

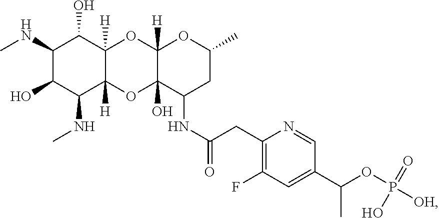 [SMILES: CN[C@@H]1[C@H](O)[C@H](NC)[C@H]2O[C@@]3(O)C(NC(=O)Cc4ncc(C(C)OP(=O)(O)O)cc4F)C[C@@H](C)O[C@H]3O[C@@H]2[C@H]1O]

or a subgroup thereof.

Aspect 119. The compound of Aspect 1, present as:

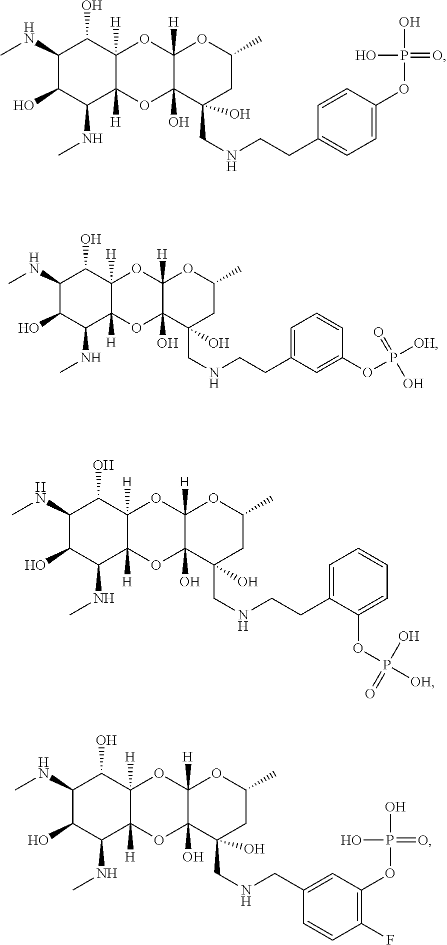 [SMILES: CN[C@@H]1[C@H](O)[C@H](NC)[C@H]2O[C@]3(O)[C@H](O[C@@H]2[C@H]1O)O[C@H](C)C[C@@]3(O)CNCCc1ccc(OP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCCc1cccc(OP(=O)(O)O)c1 ; CN[C@@H]1[C@H](O)[C@H](NC)[C@H]2O[C@]3(O)[C@H](O[C@@H]2[C@H]1O)O[C@H](C)C[C@@]3(O)CNCCc1ccccc1OP(=O)(O)O ; CN[C@@H]1[C@H](O)[C@H](NC)[C@H]2O[C@]3(O)[C@H](O[C@@H]2[C@H]1O)O[C@H](C)C[C@@]3(O)CNCc1ccc(F)c(OP(=O)(O)O)c1]

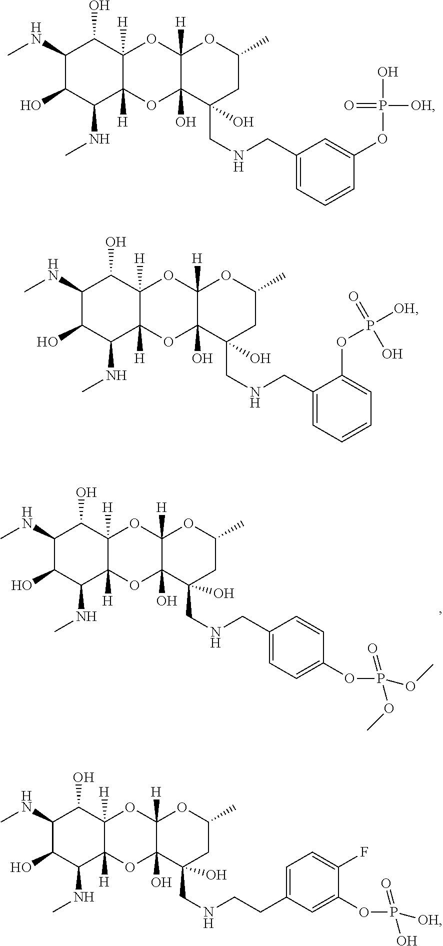

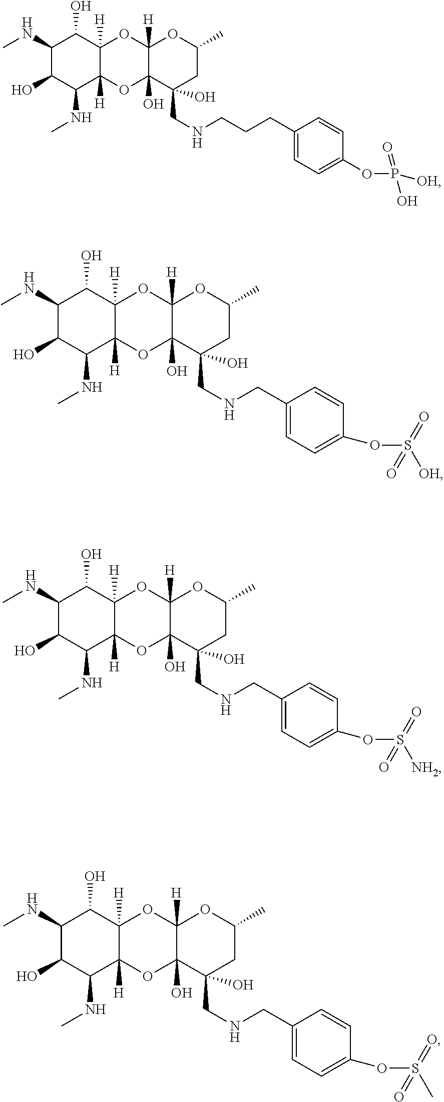 [SMILES: CN[C@@H]1[C@H](O)[C@H](NC)[C@H]2O[C@]3(O)[C@H](O[C@@H]2[C@H]1O)O[C@H](C)C[C@@]3(O)CNCCCc1ccc(OP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(=O)(=O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(N)(=O)=O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OS(C)(=O)=O)cc1]

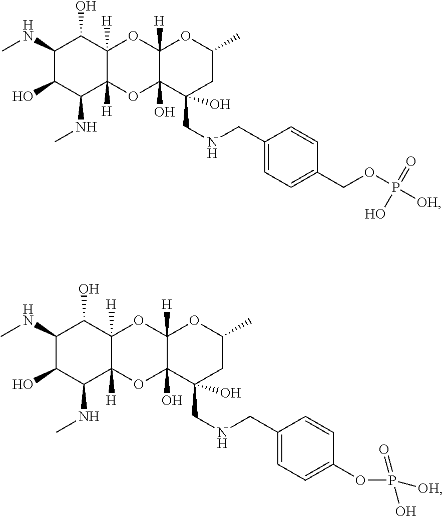 [SMILES: CN[C@@H]1[C@H](O)[C@H](NC)[C@H]2O[C@]3(O)[C@H](O[C@@H]2[C@H]1O)O[C@H](C)C[C@@]3(O)CNCc1ccc(COP(=O)(O)O)cc1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(OP(=O)(O)O)cc1]

or a subgroup thereof.

Aspect 120. A pharmaceutical composition comprising a therapeutically effective amount of a compound of any of Aspect 1-Aspect 119, or a pharmaceutically acceptable salt thereof, and a pharmaceutically acceptable carrier.

Aspect 121. The pharmaceutical composition of Aspect 120, wherein the compound has a structure represented by a formula:

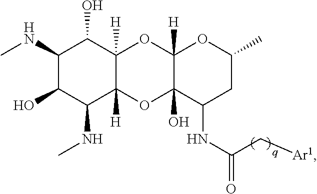

wherein q is an integer selected from 0, 1, 2, and 3.

Aspect 122. The pharmaceutical composition of Aspect 121, wherein the compound has a structure represented by a formula:

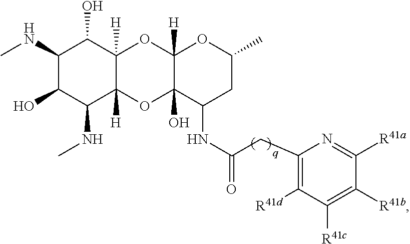

wherein 1 of R^{41a}, R^{41b}, R^{41c}, and R^{41d} is R²⁰; wherein 1, 2, or 3 of R^{41a}, R^{41b}, R^{41c}, and R^{41d} are independently hydrogen; and, wherein 0, 1, or 2 of R^{41a}, R^{41b}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

Aspect 123. The pharmaceutical composition of Aspect 122, wherein the compound has a structure represented by a formula:

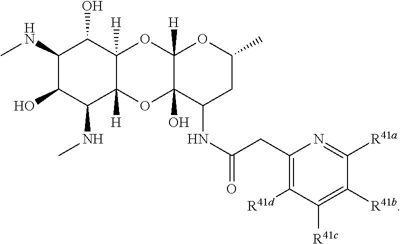

Aspect 124. The pharmaceutical composition of Aspect 120, wherein the compound has a structure represented by a formula:

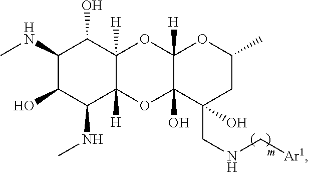

wherein m is an integer selected from 0, 1, 2, and 3.

Aspect 125. The pharmaceutical composition of Aspect 124, wherein the compound has a structure represented by a formula:

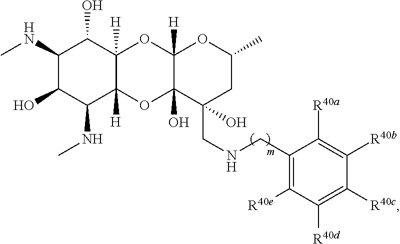

wherein 1 of R^{40a}, R^{40b}, R^{40c}, R^{40d}, and R^{40e} is R²⁰; wherein 2, 3, or 4 of R^{40a}, R^{40b}, R^{40c}, R^{40d}, and R^{40e} are independently hydrogen; and, wherein 0, 1, or 2 of R^{40a}, R^{40b}, R^{40c}, R^{40d}, and R^{40e} are independently selected from halo, cyano, hydroxyl, —NH₂, C1-C3 alkyl, C1-C3 haloalkyl, C1-C3 alkoxy, and C1-C3 haloalkoxy.

Aspect 126. The pharmaceuticai composition of Aspect 125, wherein the compound has a structure represented by a formula:

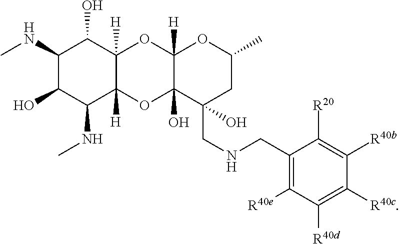

Aspect 127. The pharmaceutical composition of Aspect 125, wherein the compound has a structure represented by a formula:

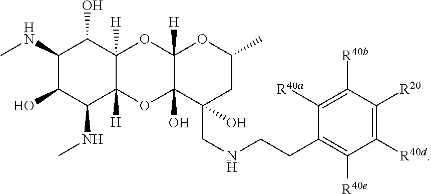

Aspect 128. The pharmaceutical composition of any one of Aspect 120-Aspect 127, wherein the pharmaceutical composition is a solid dosage form selected from a capsule, a tablet, a pill, a powder, a granule, an effervescing granule, a gel, a paste, a troche, and a pastille.

Aspect 129. The pharmaceutical composition of any one of Aspect 120-Aspect 127, wherein the pharmaceutical composition is a liquid dosage form selected from an emulsion, a solution, a suspension, a syrup, and an elixir.

Aspect 130. The pharmaceutical composition of any one of Aspect 120-Aspect 129, further comprising a second active agent.

Aspect 131. The pharmaceutical composition of Aspect 130, wherein the second active agent is an antibacterial agent.

Aspect 132. The pharmaceutical composition of Aspect 131, wherein the antibacterial agent comprises a compound selected from amoxicillin, ampicillin, azithromycin, aztreonam, azlocillin, bacitracin, carbenicillin, cefaclor, cefadroxil, cefamandole, cefazolin, cephalexin, ceidinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, dalbavancin, dalfopristin, demeclocycline, dicloxacillin, dirithromycin, doxycycline, erythromycin, enrofloxacin, enoxacin, enviomycin, ertepenem, ethambutol, flucloxacillin, fosfomycin, furazolidone, gatifloxacin, gentamicin, imipenem, isoniazid, kanamycin, linezolid, lomefloxacin, loracarbef, mafenide, moxifloxacin, meropenem, metronidazole, mezlocillin, minocycline, mupirocin, nafcillin, nalidixic acid, neomycin, netilrnicin, nitrofurantoin, norfloxacin, ofloxacin, oritavancin, oxytetracycline, penicillin, piperacillin, platensimycin, polymixin B, quinupristin, retapamulin, rifabutin, rifampin, rifapentine, roxithromycin, sparfloxacin, spectinomycin, sulbactam, sulfacetamide, sulfarnethizole, sulfarnethoxazole, teicoplanin, telithromycin, telavancin, temafloxacin, tetracycline, thioacetazone, thioridazine, ticarcillin, tinidazole, tobramycin, torezolid, tosufloxacin, trimethoprim, troleandomycin, trovafloxacin, and vancomycin, or combinations thereof.

Aspect 133. A method for the treatment of an infectious disease in a human subject comprising the step of administering to a subject a therapeutically effective amount of at least one compound of any of Aspect 1-Aspect 119, ora pharmaceutically acceptable salt thereof; or a pharmaceutical composition of any one of Aspect 120-Aspect 132.

Aspect 134. The method of Aspect 133, wherein the subject has been diagnosed with a need for treatment of the infectious disease prior to the administering step.

Aspect 135. The method of Aspect 133, further comprising the step of identifying a subject in need of treatment of the infectious diesease.

Aspect 136. The method of any one of Aspect 133-Aspect 135, wherein the infectious disease is associated with a Mycobacterium sp. infection.

Aspect 137. The method of Aspect 136, wherein the Mycobacterium sp. is selected from M. tuberculosis, M, avium intracellulare, M. abscesses, M. kansasii, M. fortuitum, M. chelonae, and M. leprae.

Aspect 138. The method of Aspect 137, wherein the M. tuberculosis is M. tuberculosis complex comprising one or more of M. tuberculosis sense stricto, M. africanum, M. canetti, M. bovis, M. caprae, M. microti, M. pinnipedii, M. mungi, and M. orygis.

Aspect 139. The method of any one of Aspect 133-Aspect 136, wherein the infectious disease is associated with a Gram positive bacterial infection.

Aspect 140. The method of Aspect 139, wherein the Gram positive bacteria is selected from Bacillus sp. Clostridium sp., Enterococcus sp., Corynebacterium sp, Staphylococcus sp., and Streptococcus sp.

Aspect 141. The method of Aspect 140, wherein the Gram positive bacteria is vancomycin resistant Enterococcus sp. (VRE).

Aspect 142. The method of Aspect 140, wherein the Gram positive bacteria is methicillin resistant Staphylococcus sp. (MRS).

Aspect 143. The method of Aspect 140, wherein the Gram positive bacteria is selected from Bacillus anthracis, Bacillus cereus, Bacillus subtilis, Clostridium difficile, Clostridium tetani, Clostridium botulinum, Clostridium perfringens, Corynebacterium diphtheria, Enterococcus faecalis, Enterococcus faecium, Listeria monocytogenes, Listeria ivanovii, Micrococcus luteus, Staphylococcus aureus, Staphylococcus epidermidis, Staphylococcus saprophyticus, Staphylococcus hyicus, Staphylococcus intermedius, Streptococcus pneumoniae, and Streptococcus pyogenes.

Aspect 144. The method of Aspect 140, wherein the Gram positive bacteria is selected from Bacillus anthracis, Bacillus subtilis, Enterococcus faecalis, Staphylococcus aureus, Streptococcus pneumoniae, and Streptococcus pyogenes.

Aspect 145. The method of any Aspect 139, wherein the Gram positive bacteria is selected from vancomycin resistant Enterococcus faecalis, vancomycin resistant methicillin resistant Enterococcus faecium, Staphylococcus aureus (MRSA), methicillin resistant Staphylococcus epidermidis (MRSE), macrolide resistant Streptococcus pneumoniae (Mac-R SPN) and penicillin resistant Streptococcus pneumoniae (PRSP).

Aspect 146. The method of any one of Aspect 133-Aspect 136, wherein the infectious disease is associated with a Gram negative bacterial infection.

Aspect 147. The method of Aspect 146, wherein the Gram negative bacteria is selected from Acinetobacter sp., Aeromonas sp., Burkholderia sp., Bordetella sp., Citrobacter sp., Enterobacter sp., Escherichia sp., Francisella sp., Haemophilus sp., Klebsiella sp., Legionella sp., Moraxella sp., Neisseria sp., Proteus sp., Pseudomonas sp., Salmonella sp., Shigella sp., Stenotrophomonas sp., Vibrio sp., and Yersinia sp.

Aspect 148. The method of Aspect 146, wherein the Gram negative bacteria is selected from Acinetobacter baumannii, Aeromonas hydrophila, Bordetella pertussis, Bordetella parapertussis, Bordetella bronchiseptica, Burkholderia cepacia, Citrobacter freundii, Enterobacter aerogenes, Enterobacter cloacae, Enterobacter sakazakii. Escherichia coli, Francisella tularensis, Haemophilus influenzae, Haemophilus aegypticus, Haemophilus ducreyi, Klebsiella edwardsii, Klebsiella pneumoniae, Legionella pneumophilia, Moraxella catarrhalis, Neisseria meningitidis, Neisseria gonorrhoeae, Proteus mirabilis, Proteus vulgaris, Pseudomonas aeruginosa, Salmonella enterica, Shigella boydii, Shigella dysenteriae, Shigella flexneri, Shigella sonnei, Stenotrophomonas maltophilia, Vibrio cholerae, Vibrio parahaemolyticus, Vibrio vulnificus, Vibrio fluvialis, Yersinia pestis. Yersina enterocolitica, and Yersina pseudotuberculosis.

Aspect 149. The method of Aspect 146, wherein the Gram negative bacteria is selected from Acinetobacter baumannii, Burkholderia cepacia, Escherichia coli, Francisella tularensis, Haemophilus influenza, Klebsiella pneumonia, Legionella pneumophilia, Neisseria gonhorrhoeae, Neisseria meningitides. Pseudomonas aeruginosa, Proteus mirabilis, Proteus vulgaris, Stenotrophomonas maltophilia, and Yersinia pestis.

Aspect 150. The method of Aspect 146, wherein the Gram negative bacteria is an Escherichia coli strain with a ΔtolC mutation.

Aspect 151, The method of Aspect 146, wherein the Gram negative bacteria is a multi-drug resistant Gram negative bacteria strain (MDR-GNB).

Aspect 152. The method of Aspect 151, wherein the multi-drug resistant Gram negative bacteria strain (MDR-GNB) is resistant to at least one anti-microbial agent selected from amikacin, tobramycin, cefepime, ceftazidime, Imipenem, meropenem, piperacillin-tazobactam, ciprofloxacin, levofloxacin, tigecycline, and polymyxin B.

Aspect 153, The method of Aspect 151 or Aspect 152, wherein the multi-drug resistant Gram negative bacteria strain (MDR-GNB) is selected from Acinetobacter sp., Enterobacter sp., Klebsiella sp., and Pseuodomonas sp.

Aspect 154. The method of any of Aspect 151-Aspect 153, wherein the multi-drug resistant Gram negative bacteria strain (MDR-GNB) is selected from Acinetobacter baurnannii, Enterobacter aerogenes, Klebsiella pneumoniae, and Pseudomonas aeruginosa.

Aspect 155. The method of any of Aspect 151-Aspect 154, wherein the mufti-drug resistant Gram negative bacteria strain (MDR-GNB) is Enterobacter sp.

Aspect 156. The method of any one of Aspect 133-Aspect 155, wherein the infectious disease is selected from tuberculosis, bacterial meningitis, cholera, dental infection, dermatitis, diarrhea, diphtheria, dysentery, ear infection, endocarditis,gastritis, gastroenteritis, genital infection, genitourinary infection, infection associated with an indwelling device. intestinal infection, leprosy, listeriosis, lung infection, nocosomial infection, ocular infection, oral infection, otitis, osteo-articular infection, osteomyelitis, pharyngitis, papules, pharyngitis, pneumonia conjunctivitis, pruritius, pustules, pyoderma, pyothorax, respiratory infection, salmonellosis, septicemia, sexually transmitted disease, sinusitis, skin infection, skin and soft tissue infection (“SSTI”). soft tissue infection, tetanus, ulcer, urinary tract infection, and wound infection.

Aspect 157. The method of Aspect 156, wherein the infectious disease is selected from endocardititis, osteomyelitis, skin and soft tissue infection (“SSTI”), and infection associated with an indwelling device.

Aspect 158. The method of Aspect 156, wherein the infectious disease is endocardititis.

Aspect 159. The method of Aspect 156, wherein the infectious disease is osteomyelitis.

Aspect 160. The method of Aspect 156, wherein the infectious disease is an SSTI.

Aspect 161. The method of Aspect 160, wherein the SSTI is a complicated SSTI (cSSTI).

Aspect 162. The method of Aspect 156, wherein the infectious disease is associated with an indwelling device.

Aspect 163. The method of any one of Aspect 133-Aspect 162, further comprising administering to the human subject a therapeutically effective amount of a second active agent.

Aspect 164. The method of Aspect 163, wherein the second active agent comprises an immunomodulator.

Aspect 165. The method of Aspect 164, wherein the imrnunomodulator is a cytokine, an interleukin, a chemokine, or combinations thereof.

Aspect 166. The method of Aspect 165, wherein the immunomodulatory is selected from 1L-2, IL-7 and 1L-12, IFN-α, 1FN-β, IFN-ε, IFN-κ, IFN-ω, IFN-γ, IFN-γ1b, CCL3. CCL26, CXCL7, and combinations thereof.

Aspect 167. The method of Aspect 163, wherein the second active agent comprises at least one anti-tuberculosis agent.

Aspect 168. The method of Aspect 167, wherein the anti-tuberculosis agent is selected from amikacin, amoxicillin-clavulanic acid, bedaquiline, capreomycin, ciprofloxacin, clarithromycin, clofazimine, cycloserine, delamanid, ethambutol, ethionamide,gatifloxacin, imipenem, isoniazid, kanamycin, levofloxacin, linezolid, meropenem, moxifloxacin, ofloxacin, para-aminosalicylic acid, pretornanid, pyrazinamide, rifampin, rifapentine, rifabutin, SQ109, streptomycin, sudoterb, terizidone, thiacetazone, viomycin, and combinations thereof,

Aspect 169. The method of Aspect 163, wherein the second active agent comprises at least one antibacterial agent.

Aspect 170. The method of Aspect 169, wherein the antibacterial agent comprises a compound selected from amoxicillin, ampicillin, azithromycin, aztreonam, aziocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, dalbavancin, dalfopristin, demeclocycline, dicloxacillin, dirithromycin, doxycycline, erythromycin, enrofloxacin, enoxacin, enviornycin, ertepenem, ethambutol, flucloxacillin, fosfomycin, furazolidone, gatifloxacin, gentamicin, imipenem, isoniazid, kanamycin, linezolid, lomefloxacin, loracarbef, mafenide, moxifloxacin, meropenem, metronidazole, mezlocillin, minocycline, mupirocin, nafcillin, nalidixic acid, neomycin, netilmicin, nitrofurantoin, norfloxacin, ofloxacin, oritavancin, oxytetracycline, penicillin, piperacillin, platensimycin, polymixin B, quinupristin, retapamulin, rifabutin, rifampin, rifapentine, roxithromycin, sparfloxacin, spectinomycin, sulbactam, sulfacetamide, sulfamethizole, sulfamethoxazole, teicoplanin, telithrornycin, telavancin, temafloxacin, tetracycline, thioacetazone, thioridazine, ticarcillin, tinidazole, tobramycin, torezolid, tosufloxacin, trimethoprim, troleandomycin, trovafloxacin, and vancomycin, or combinations thereof.

Aspect 171. The method of any one of Aspect 163-Aspect 170, wherein the administering is co-administering of the compound and the second active agent.

Aspect 172. The method of Aspect 171, wherein the co-administration is administration in a substantially simultaneous manner.

Aspect 173. The method of Aspect 172, wherein the administration in a substantially simultaneous manner comprises a single dose form containing a fixed ratio of the compound and the antibacterial agent.

Aspect 174. The method of Aspect 173, wherein the single dose form is a capsule or a tablet.

Aspect 175. The method of Aspect 173, wherein the single dose form is an ampule for a single intravenous administration,

Aspect 176. The method of Aspect 171, wherein the co-administration is administration in a substantially sequential manner.

Aspect 177. A method for inhibiting protein synthesis in at least one bacterial cell, comprising the step of contacting the at least one bacterial cell with an effective amount of at least one compound of any of 1-Aspect 119, or a pharmaceutically acceptable salt thereof; or a pharmaceutical composition of any one of Aspect 120-Aspect 132.

Aspect 178, The method of Aspect 177, wherein the bacterial cell is a Gram positive bacterial cell.

Aspect 179. The method of Aspect 177, wherein the bacterial cell is a Gram negative bacterial cell.

Aspect 180. The method of any of Aspect 177-Aspect 179, wherein the contacting is via administration to a mammal.

Aspect 181. The method of Aspect 180, wherein the mammal has been diagnosed with a need for treatment of an infectious disease prior to the administering step,

Aspect 182. The method of Aspect 180, wherein the mammal has been diagnosed with a need for inhibiting protein synthesis in the bacterial cell prior to the administering step.

Aspect 183. A kit comprising at least one compound of any of Aspect 1-Aspect 119, or a pharmaceutically acceptable salt thereof; or a pharmaceutical composition of any one of Aspect 120-Aspect 132; and one or more of: (a) at least one agent known to microbial ribosomal activity; (b) at least one agent known to have antimicrobial activity; (c) at least one agent known to treat an infectious disease; (d) instructions for treating an infectious disease; (e) instructions for administering the compound in connection with treating a microbial infection; or (f) instructions for administering the compound with at least one agent known to treat an infectious disease.

Aspect 184. The kit of Aspect 183, wherein the compound and the agent are co-formulated,

Aspect 185. The kit of Aspect 183, wherein the compound and the agent are co-packaged.

Aspect 186. The kit of any of Aspect 183-Aspect 185, further comprising a plurality of dosage forms, the plurality comprising one or more doses; wherein each dose comprises an effective amount of the compound and the agent known to have antimicrobial activity.

Aspect 187. The kit of Aspect 186, wherein an effective amount is a therapeutically effective amount.

Aspect 188. The kit of Aspect 186, wherein an effective amount is a prophylatically effective amount.

Aspect 189. The kit of any of Aspect 186-Aspect 188, wherein each dose of the compound and the agent known to have antimicrobial activity are co-formulated.

Aspect 190. The kit of any of Aspect 186-Aspect 188, wherein each dose of the compound and the agent known to have antimicrobial activity are co-packaged,

Aspect 191, The kit of any of Aspect 186-Aspect 190, wherein the dosage forms are formulated for oral administration and/or intravenous administration.

Aspect 192. The kit of Aspect 191, wherein the dosage forms are formulated for oral administration.

Aspect 193. The kit of Aspect 191, wherein the dosage forms are formulated for intravenous administration.

Aspect 194. The kit of any of Aspect 186-Aspect 190, wherein the dosage form for the compound is formulated for oral administration and the dosage form for the agent known to have antimicrobial activity is formulated for intravenous administration.

Aspect 195. The kit of any of Aspect 186-Aspect 190, wherein the dosage form for the compound is formulated for intravenous administration and the dosage form for the agent known to have antimicrobial activity is formulated for oral administration.

Aspect 196, The kit of any of Aspect 183-Aspect 195, wherein the agent known to have antimicrobial activity is an anti-tuberculosis agent.

Aspect 197. The kit of Aspect 196, wherein the anti-tuberculosis agent is selected from amikacin, amoxicillin-clavulanic acid, bedaquiline, capreomycin, ciprofioxacin, clarithromycin, clofazimine, cycloserine, delamanid, ethambutol, ethionarnide,gatifloxacin, imipenem, isoniazid, kanamycin, levofloxacin, linezolid, meropenem, moxifloxacin, ofloxacin, para-aminosalicylic acid, pretomanid, pyrazinamide, rifampin, rifapentine, rifabutin, SQ109, streptomycin, sudoterb, terizidone, thiacetazone, viomycin, and combinations thereof.

Aspect 198. The kit of any of Aspect 183-Aspect 195, wherein the agent known to have antimicrobial activity is an antibacterial agent.

Aspect 199. The kit of Aspect 198, wherein the antibacterial agent comprises a compound selected from amoxicillin, ampicillin, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, dalbavancin, dalfopristin, demeclocycline, dicloxacillin, dirithromycin, doxycycline, erythromycin, enrofloxacin, enoxacin, enviomycin, ertepenem, ethambutol, flucloxacillin, fosfomycin, furazolidone, gatifloxacin, gentamicin, imipenem, isoniazid, kanamycin, linezolid, lomefloxacin, loracarbef, mafenide, moxifloxacin, meropenem, metronidazole, mezlocillin, minocycline, mupirocin, nafcillin, nalidixic acid, neomycin, netilmicin, nitrofurantoin, norfloxacin, ofloxacin, oritavancin, oxytetracycline, penicillin, piperacillin, platensimycin, polymixin B, quinupristin, retapamulin, rifabutin, rifampin, rifapentine, roxithromycin, sparfloxacin, spectinomycin, sulbactam, sulfacetamide, sulfamethizole, sulfamethoxazole, teicoplanin, telithromycin, telavancin, temafloxacin, tetracycline, thioacetazone, thioridazine, ticarcillin, tinidazole, tobramycin, torezolid, tosufloxacin, trimethoprim, troleandomycin, trovafloxacin, and vancomycin, or combinations thereof.

Aspect 200, The kit of any of Aspect 183-Aspect 199, wherein the instructions for treating an infectious disease provide for treatment of a Gram positive bacterial infection.

Aspect 201. The kit of any of Aspect 183-Aspect 199, wherein the instructions for treating an infectious disease provide for treatment of a Gram negative bacterial infection.

Aspect 202. The kit of any of Aspect 183-Aspect 201, wherein the instructions for treating an infectious disease provide for treatment of an infectious disease selected from tuberculosis, bacterial meningitis, cholera, dental infection, dermatitis, diarrhea, diphtheria, dysentery, ear infection, endocarditis,gastritis, gastroenteritis, genital infection, genitourinary infection, infection associated with an indwelling device, intestinal infection, leprosy, listeriosis, lung infection, nocosomial infection, ocular infection, oral infection, otitis, osteo-articular infection, osteomyelitis, pharyngitis, papules, pharyngitis, pneumonia conjunctivitis, pruritius, pustules, pyoderma, pyothorax, respiratory infection, salmonellosis, septicemia, sexually transmitted disease, sinusitis, skin infection, skin and soft tissue infection (“SSTI”), soft tissue infection, tetanus, ulcer, urinary tract infection, and wound infection.

Aspect 203. The kit of Aspect 202, wherein the instructions for treating an infectious disease provide for treatment of an infectious disease selected tuberculosis, endocardititis, osteomyelitis, skin and soft tissue infection (SSTI), and infection associated with an indwelling device.

Aspect 204. The kit of Aspect 203, wherein the infectious disease is tuberculosis.

Aspect 205. The kit of Aspect 203, wherein the infectious disease is endocardititis.

Aspect 206. The kit of Aspect 203, wherein the infectious disease is osteomyelitis.

Aspect 207. The kit of Aspect 203, wherein the infectious disease is an SSTI.

Aspect 208. The kit of Aspect 207, wherein the SSTI is a complicated SSTI (cSSTI),

Aspect 209. The kit of Aspect 203, wherein the infectious disease is associated with an indwelling device.

Aspect 210. The kit of any of Aspect 183-Aspect 209, wherein the instructions for administering the compound with at least one agent known to treat an infectious disease provide for co-administering of the compound and the agent.

Aspect 211. The kit of Aspect 210, wherein the co-administration is administration in a substantially simultaneous manner.

Aspect 212. The kit of Aspect 211, wherein the administration in a substantially simultaneous manner comprises a single dose form containing a fixed ratio of the compound and the antibacterial agent.

Aspect 213. The kit of Aspect 212, wherein the single dose form is a capsule or a tablet.

Aspect 214. The kit of Aspect 212, wherein the single dose form is an ampule for a single intravenous administration.

Aspect 215. The kit of Aspect 210, wherein the co-administration is administration in a substantially sequential manner.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. 2-(5-hydroxypyridin-2-yl)-N-((2R,4R,4aS,5aR,6S,7S,8R,9S,9aR,10aS)-4a,7,9-trihydroxy-2-methyl-6,8-bis(methylamino)decahydro-2H-benzo[b]pyrano[2,3-e] [1,4]dioxin-4-yl)acetamide (Compound 1810)

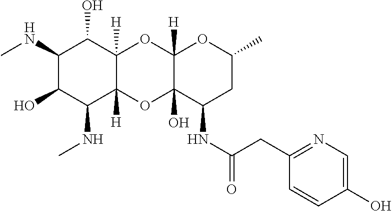 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1ccc(O)cn1]

The synthetic scheme to prepare he above-referenced compound from spectinomycin is shown immediately below.

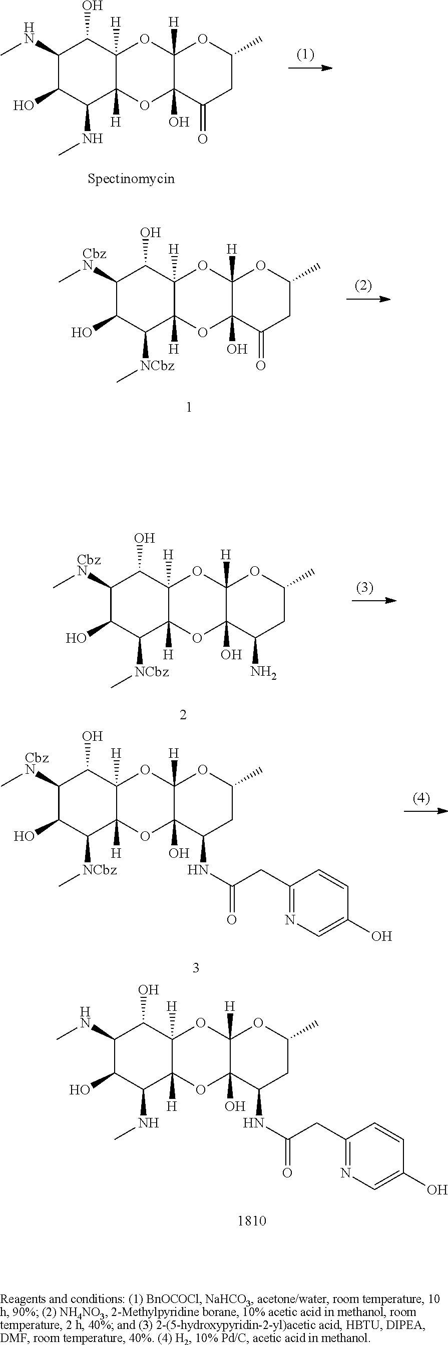

Synthesis of Compound 1: Bis-Cbz-spectinomycin was synthesized from spectinomycin following the previously reported protocol of P. M. Herrinton, K. L. Klotz and W. M. Hartley (J Org Chem, 1993. 58, 678-682). The target compound was obtained in a 300 g batch and about a 90% yield.

Synthesis of Compound 2: the synthesis was based on a previously described method (J. Liu, et al., ACS Infect. Dis, 2017, 3, 72-88). Alternatively, the synthesis of compound 2 can follow the procedure as described in R.E.Lee et al Nat Med. 2014 February; 20(2):152-158. Briefly, Compound 1 (200 g, 333 mmol), NH₄NO₃ (267 g, 3330 mmol, 10 eq) and acetic acid (240 g) were mixed into MeOH (2000 mL). After the mixture was stirred at room temperature around 20 mins, 2-Methylpyridine borane (21 g, 196 mmol, 0.6 eq) was added into the solution in one portion. The reaction was monitored by UPLC, the reaction is finished around an hour. The solvent was then removed with rotavap. The residue was dispersed into water (1L) and ethyl acetate (EA) (750 mL). The EA layer was separated, and the aqueous layer was extracted twice with EA (30 mL each).

The combined EA layer was extracted by 0.1 M HCl (500 mL) once and C.1 M HCl (250 mL) 6 times. After 2 days, the combined aqueous mixed with EA 500 mL, which was then neutralized by NaHCO₃. The mixture was shaken well and then keep it in the separating funnel for around an hour until the clear solution turn into milky solution. The precipitate was filtered and then transfer the solid into EA. Stirring the EA mixture for an hour and then filter it again. The filtration contains most of s isomer. Dissolved the solid with MeOH 300 mL and then partitioned between EA and saturated NaHCO₃. The aqueous layer was extracted by EA twice. The combined EA layer was washed by brine once and dried over NaSO₄, EA was removed and compound 2 was obtained as light yellow solid (40g). It was ready to be used directly in the next step.

Synthesis of Compound 3: 2-(5-hydroxypyrldin-2-yl)acetic acid (6.6 g, 35 mmol, 1 eq), compound 2 (21.0 g, 35 mmol, 1 eq), HBTU (13.3 g, 35 mmol, 1.05 eq) and triethylamine (TEA, 13.6 g, 105 mmol, 3 eq) were mixed in DMF (200 mL) all at once. The reaction mixture was quenched by water in 5-30 min (about the time the reaction mix is clear and monitored by LC/MS). Then the solution was extracted by EA (500 mL) twice, the combined EA layer was washed by brine, dried over Na₂SO₄. After the solvent was removed, the residue was purified by C18 chromatography. (biotage system, 400 g size of column, began with 1 column volume (CV) of water, 4 CV from C%-40% acetonitrile/water, keep 40% until all compound 3 was flushed out). After solvent was removed from collections, treat the residue with EA, Compound 3 will be precipitated from the solution after 10-20 hs. Compound 3 was collected and no further purification needed, (If necessary, retreat the solid with EA until the compound is pure enough). Yield 8.5g (33%) white solid.

Synthesis of Compound 1810: Compound 3 (1.0 g, 1.35 mmol) was mixed with 10% Pd/C (0.1g) and C.5 M HCl in MeOH (10 mL), the mixture was hydrogenated at room temperature for 2 hours. Pd/C was removed and then the filtrate was dried under vacuum to yield 0.7 g (90%) compound 1810 as a white solid. ¹H NMR (400 MHz, Deuterium Oxide) δ 8.04 (dd, J=C0.8, 2.9 Hz, 1H), 7.34-7.25 (m, 2H), 4.97 (5, 1H), 4.59 (t, J=2.9 Hz, 1H), 4.32-4.22 (m, 1H), 4.16 (t, J=3.2 Hz, 1H), 4.11-3.73 (m, 5H), 3.14 (dd, J=2.7, 10.9 Hz, 1H), 3.01 (dd, J=2.8, 10,3 Hz, 1H), 2.70 (5, 3H), 2.62 (s, 3H), 1.93-1.85 (m, I H), 1.77 (dt, J=2.4, 14.3+Hz, 1H), 1.26 (d, J=6.08 Hz, 3H). HRMS-ESI calcd for C₂₁H₃₃N₄O₈, [M+H⁺] 469.2298, found: 469,2301.

### 2. 6-(2-oxo-2-(((2R,4R,4a5,5aR,6S,7S,8R,9S,9aR,10aS)-4a,7,9-trihydroxy-2-methyl-6,8-bis(methylamino)decahydro-2H-benzo[b]pyrano[2,3-e][1,4]dioxin-4-yl)amino)ethyl)pyridin-3-yl dihydrogen phosphate (Compound 3408)

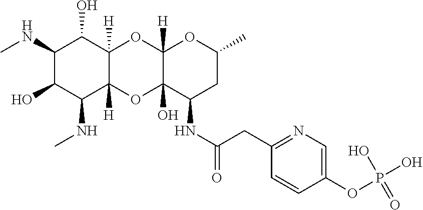 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1ccc(OP(=O)(O)O)cn1]

The synthetic scheme to prepare the above-referenced compound starting from compound 3 is shown immediately below.

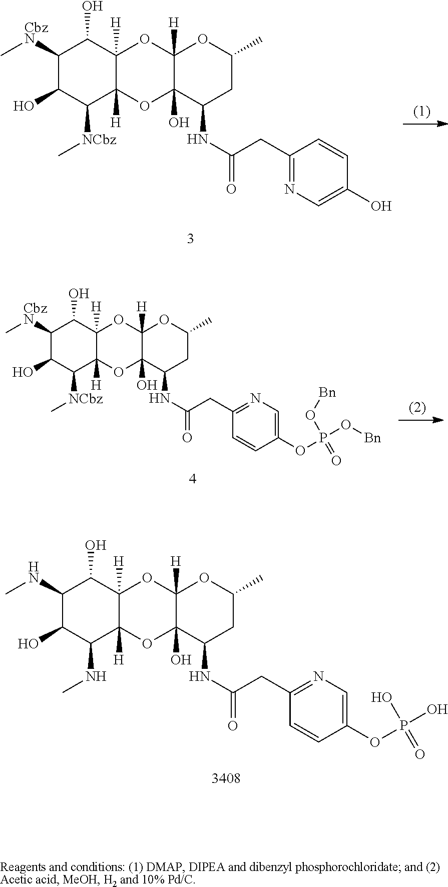

Synthesis of Compound 4: To a stirred solution of trichloroisocyanuric acid (0.87 g, 3.73 mmol) in dry acetonitrile (20 mL) at room temperature was added in one portion of diisopropyl phosphite, (2.96 g, 11.29 mmol). The resulting mixture was stirred at room temperature until cyanuricacid precipitated from the reaction mixture. The reaction mixture was settled down before decanting the clear solution to a round bottom flask for next step. Compound 3 (6.40 g 8.69 mmol) was added to the solution above, followed by N,N-diisopropylethylamine (2.25 g, 17.37 mmol) and N,N-dimethylpyridin-4-amine (0.11 g, 0.87mmol). The mixture was stirred at room temperature and monitored by UPLC, after 1 h, the reaction mixture was partitioned between EA and water. The organic layer was washed with brine and dried over Na₂SO₄. After removing solvent, the residue was purified by chromatography (EA/MeOH) and 5.8 g (67%) white solid was obtained.

Synthesis of Compound 3408: Compound 4 (7.0 g, 7.02 mmol) was mixed with 10% Pd/C (0.7 g) and acetic acid (1.7 g, 28 mmol) in MeOH (30 mL), the mixture was hydrogenated at room temperature for 8 hs. Pd/C was removed and then the filtrate was dried under vacuum to yield 3.2 g (83%) final phosphate as white solid. ¹H NMR (400 MHz, Deuterium Oxide) δ 8.35 (d, J=2,8 Hz, 1H), 7.80-7.63 (m, 11-1), 7.40 (d, J=8.6 Hz, 1H), 5.01 (s, 1H), 4.52-4.39 (m, 1H), 4.21-3.79 (m, 6H), 3.51 (dd, J=11.1, 2.8 Hz, 1H), 3.26 (dd, J=10.2, 2.8 Hz, 1H), 2.84 (d, J=5.1 Hz, 6H), 1.93-1.84 (m, 1H), 1.84-1.73 (m, 1H), 1.32-1.22 (m, 4H). HRMS-ESI calcd for C₂₁H₃₄N₄O₁₁P [M+H]⁺549.1962, found: 549.1964.

### 3. Diethyl (6-(2-oxo-2-(((2R,4R,4aS, 5aR,6S,7S,8R,9S,9aR,10aS)-4a,7,9-trihydroxy-2-methyl-6,8-bis(methylamino)decahydro-2H-benzo[b]pyrano[2,3-e][1,4]dioxin-4-yl)amino)ethyl)pyridin-3-yl) phosphate (Compound 3906)

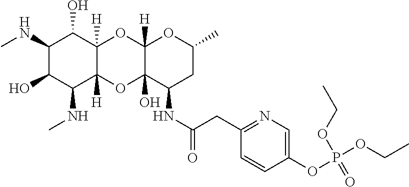 [SMILES: CCOP(=O)(OCC)Oc1ccc(CC(=O)N[C@@H]2C[C@@H](C)O[C@H]3O[C@@H]4[C@@H](O)[C@H](NC)[C@H](O)[C@H](NC)[C@H]4O[C@]32O)nc1]

The synthetic scheme to prepare the above-referenced compound starting from compound 3 is shown immediately below.

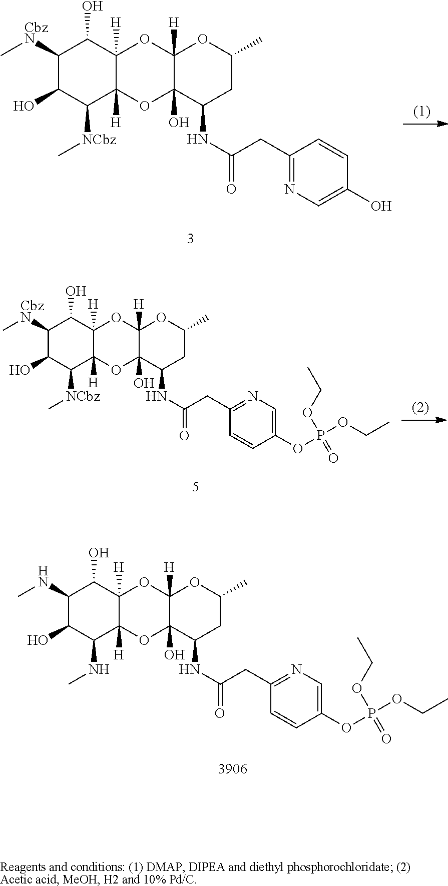

Synthesis of Compound 5: Compound 3 (500 mg, 0.679 mmol) was added to DMF solution mixture which contains N, N-diisopropylethylarnine (175 mg, 1.357 mmol) and N,N-dimethylpyridin-4-amine (8.29 mg, 0.068 mmol). Diethyl phosphorochloridate (129 mg, C.747 mmol) was added and then the mixture was stirred at room temperature and monitored by UPLC, after 2 h, the reaction mixture was partitioned between EA and water. The organic layer was washed with brine and dried over Na₂SO₄. After removing the solvent, the residue was purified by chromatography (EA/MeOH) and 350 mg (59%) compound 5 was obtained as white solid.

Synthesis of Compound 3906: Compound 5 (100 mg, 0,115 mmol) was mixed with 10% Pd/C (10 mg) and acetic acid (20 mg) in MeOH (30 mL), the mixture was hydrogenated at room temperature for 8 hs. Pd/C was removed and then the filtrate was dried under vacuum to yield 50 mg (72%) final phosphate as white solid. ¹H NMR (400 MHz, Deuterium Oxide) δ 8.59-8.32 (m, 1H), 7.75 (ddt, J=1.4, 2.9, 8.6 Hz, 1H), 7.58-7.24 (m, 1H), 4.99 (s, 1H), 4.66 (s, 1H), 4.34 (dq, J=7.1, 8.6 Hz, 5H), 4.20-3.82 (m, 6H), 3.11 (dt, J=6.1, 10.1 Hz, 1H), 2.75 (s, 2H), 2.70 (s, 2H), 1.90 (s, OH), 1.83-1.69 (m, OH), 1.37 (td, J=1.1, 7.1 Hz, 6H), 1.31-1.24 (m, 3H). ESI-MS [M+H]⁺, m/z 605.

### 4. (2R,4R,4aS,5aR,6S,7S,8R,9S,9aR,10aS)-4-(((4-hydroxyphenethyl)amino)methyl)-2-methyl-6,8-bis(methylamino)octahydro-2H-benzo[b]pyrano[2,3-e][1,4]dioxine-4,4a,7,9(10aH)-tetraol and 4-(2-((((2R,4R,4a5,5aR,65,75,8R,95,9aR,10aS)-4,4a,7,9-tetrahydroxy-2-methyl-6,8-bis(methylamino)decahydro-2H-benzo[b]pyrano[2,3-e][1,4]dioxin-4-yl)methyl)amino)ethyl)phenyl dihydrogen phosphate (Compounds 3911 and 3956, respectively)

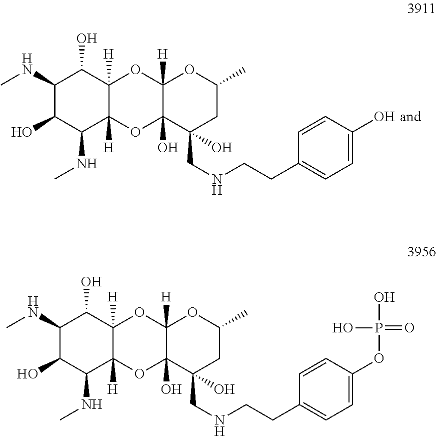 [SMILES: CN[C@@H]1[C@H](O)[C@H](NC)[C@H]2O[C@]3(O)[C@H](O[C@@H]2[C@H]1O)O[C@H](C)C[C@@]3(O)CNCCc1ccc(O)cc1 ; "and" ; CN[C@@H]1[C@H](O)[C@H](NC)[C@H]2O[C@]3(O)[C@H](O[C@@H]2[C@H]1O)O[C@H](C)C[C@@]3(O)CNCCc1ccc(OP(=O)(O)O)cc1]

The synthetic scheme to prepare the above-referenced compounds starting from spectinomycin is shown immediately below.

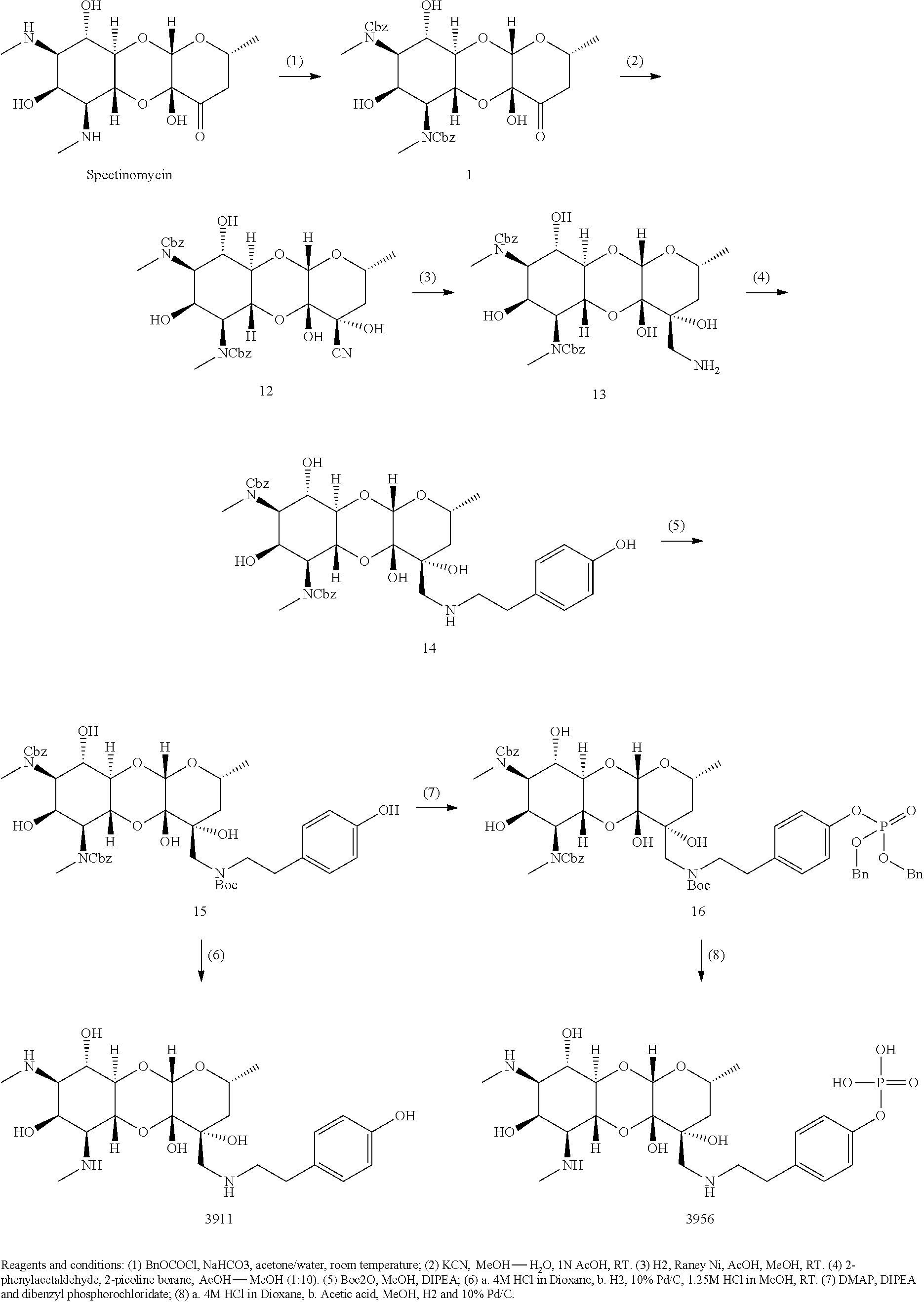

Synthesis of Compound 1: Bis-Cbz-spectinomycin was synthesized from spectinomycin as described herein above as intermediate during the preparation of 2-(5-hydroxypyridin-2-yl)-N-((2R,4R,4aS,5aR,6S,7S,8R,9S,9aR,10aS)-4a,7,9-trihydroxy-2-methyl-6,8-bis(methylamino)decahydro-2H-benzo[b]pyrano[2,3-e][1,4]dioxin-4-Aacetamide.

Synthesis of Compound 12: In 1000 ml of 2 M acetic acid in methanol was dissolved 30 g (49.9 mmol) of compound 1: To this solution was added potassium cyanide (3.25 g, 49.9 mmol) and water 50 mL. The reaction was stirred overnight at room temperature and then concentrated in vacuo. The residue was partitioned between EA and water. The EA was separated and combined with a second EA extract. The extractions were washed with brine and dried over Na₂SO₄. Removal of the solvent in vacuo afforded 31 g of a white solid.

Synthesis of Compound 13: The compound 12 was immediately taken up in 200 ml of 2 M AcOH-MeOH and hydrogenated in the presence of 30 g of Raney nickle overnight. The reaction was filtered and concentrated in vacua. The residue was partitioned between EA and 0.1 M HCl solution. The aqueous was separated and combined with water wash. The aqueous solution was made alkaline with concentrated NI-1₄0H and extracted with EA, The extracts were washed with brine and dried over Na₂SO₄. Removal of the solvent in vacuo afforded 14 g of the target compound, compound 13, as a light blue solid (46.4%)

Synthesis of Compound 14: Compound 13 (278 mg, 0.441 mmol) was dissolved into 2 M acetic acid in methanol (10 mL): To this solution, 2-phenylacetaldehyde (60 mg, 0.441 mmol) and 2-picoline borane (33mg, 0.308 mmol) were added and stirred at room temperature overnight. The methanol was removed and the residue partitioned between EA and water; and the aqueous layer was extracted with EA (2×10 mL). The combined organic layers were dried with Na₂SO₄ and concentrated under reduced pressure and purified by column chromatography to afford the target compound, 14 (163 mg, 49.2%).

Synthesis of Compound 15: Compound 14 (160 mg, 0.213 mmol) and di-tert-butyl dicarbonate (46.4 mg, 0.213 mmol) were dissovled in MeOH. After 30 minutes, the reaction mixture was partitioned between EA and water; and the the aqueous layer was extracted with EA (2×10 mL). The combined organic layers were dried with Na₂SO₄ and concentrated under reduced pressure, followed by purification using column chromatography to afford the target compound, 15 (100 mg, 55.2%).

Synthesis of Compound 3911: Compound 15 (20 mg) was dissolved in 4 M HCl in dioxane and stirred for half an hour. The solvent was removed and then the residue was mixed with 10% Pd/C (10 mg) in 1.25 M HCl in MeOH (5 mL), the mixture was hydrogenated at room temperature for overnight. Pd/C was removed and then the filtrate was dried under vacuum to yield the target compound, 3911 (17 mg; 84%). ¹H NMR (400 MHz, Deuterium Oxide) δ 7.16 (d, J=8.5 Hz, 2H), 6.90-6.78 (m, 2H), 4.81 (5, 1H), 4.28-4:16 (m, 1H), 3.94 (dt, J=9.9, 31.0 Hz, 2H), 3.52-3,37 (m, 2H), 3.28 (s, 3H), 3.25-3.16 (m, 2H), 2.99-2.90 (m, 2H), 2.76 (s, 3H), 2.76 (5, 3H), 1,86-1.63 (m, 2H), 1.18 (d, J=6.2 Hz, 3H). ESI-MS [M+H]T, m/z 484.

Synthesis of Compound 16: To a stirred solution of trichloroisocyanuric acid (77 mg, 0.33 mmol) in dry acetonitrile (6mL) at room temperature was added diisopropyl phosphite, (0.262 g; 1.0 mmol) in a single portion. The resulting mixture was stirred at room temperature until cyanuricacid precipitated from the reaction mixture, The reaction mixture was allowed to settle prior to decanting the clear solution (2 mL) into a round bottom flask for next step. Compound 15 (163 mg, C.383 mmol) was added to the preceding solution, followed by addition of N, N-diisopropylethylamine (49.5 mg, C.383 mmol) and N,N-dimethylpyridin-4-amine (2.337 mg, C.019 mmol). The mixture was stirred at room temperature and monitored by UPLC. After 1 h, the reaction mixture was partitioned between EA and water, The organic layer was washed with brine and dried over Na₂SO₄, After removing solvent, the residue was purified by chromatography (EA/MeOH) to obtain the target compound, 16 (98 mg; 46.1%).

Synthesis of Compound 3956: Compound 16 (98 mg, C.037 mmol) was dissolved in 4 M HCl in dioxane and stirred for half an hour. The solvent was removed and then the residue was mixed with 10% Pd/C (10 mg) and acetic acid (500 mg) in MeOH (5 mL), The mixture was allowed to react at room temperature for overnight. Pd/C was filtered and then the solvent was removed under vacuum to yield the target compound, 3956 (30 mg; 55.4%). ¹H NMR (400 MHz, Deuterium Oxide) δ 7.29-7.20 (m, 2H), 7.15-7.09 (m, 2H). 4.82 (5, 1H), 4.19 (dd, J=9.5, 10.9 Hz. 1H), 4.00-3.87 (m, 2H), 3,82-3.68 (m, 1H), 3,51-3.15 (m, 5H), 2.99 (tt, J=7.1, 13,7 Hz, 3H), 2.77 (5, 3H), 2.76 (s, 3H), 1,84-1.69 (m, 2H), 1.18 (d, J=6.0 Hz, 3H). ESI-MS [M+H]⁺, m/z 564.

### 5. (2R,4R,4aS,5aR,6S,7S,8R,9S,9aR,10aS)-4-(((4-fluoro-3-hydroxybenzyl)amino)methyl)-2-methyl-6,8-bis(methylamino)octahydro-2H-benzo[b]pyrano[2,3-e][1,4]dioxine-4,4a,7,9(10aH)-tetraol and 2-fluoro-5-(((((2R,4R,4aS,5aR,6S,7S,8R,9S,9aR,10aS)-4,4a,7,9-tetrahydroxy-2-methyl-6,8-bis(methylamino)decahydro-2H-benzo[b]pyrano[2,3-e][1,4]dioxin-4-yl)methyl)amino)methyl)phenyl dihydrogen phosphate (Compounds 3958 and 3959, respectively)

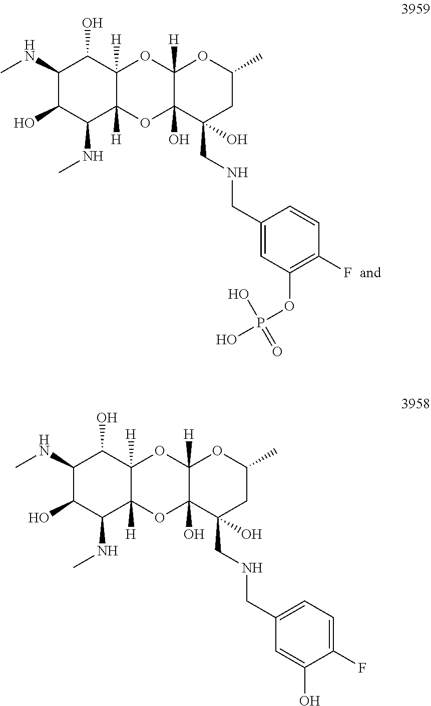 [SMILES: CN[C@@H]1[C@H](O)[C@H](NC)[C@H]2O[C@]3(O)[C@H](O[C@@H]2[C@H]1O)O[C@H](C)C[C@@]3(O)CNCc1ccc(F)c(OP(=O)(O)O)c1 ; CN[C@@H]1[C@H](O)[C@H](NC)[C@H]2O[C@]3(O)[C@H](O[C@@H]2[C@H]1O)O[C@H](C)C[C@@]3(O)CNCc1ccc(F)c(O)c1]

The synthetic scheme to prepare the above-referenced compounds starting from compound 13 is shown immediately below.

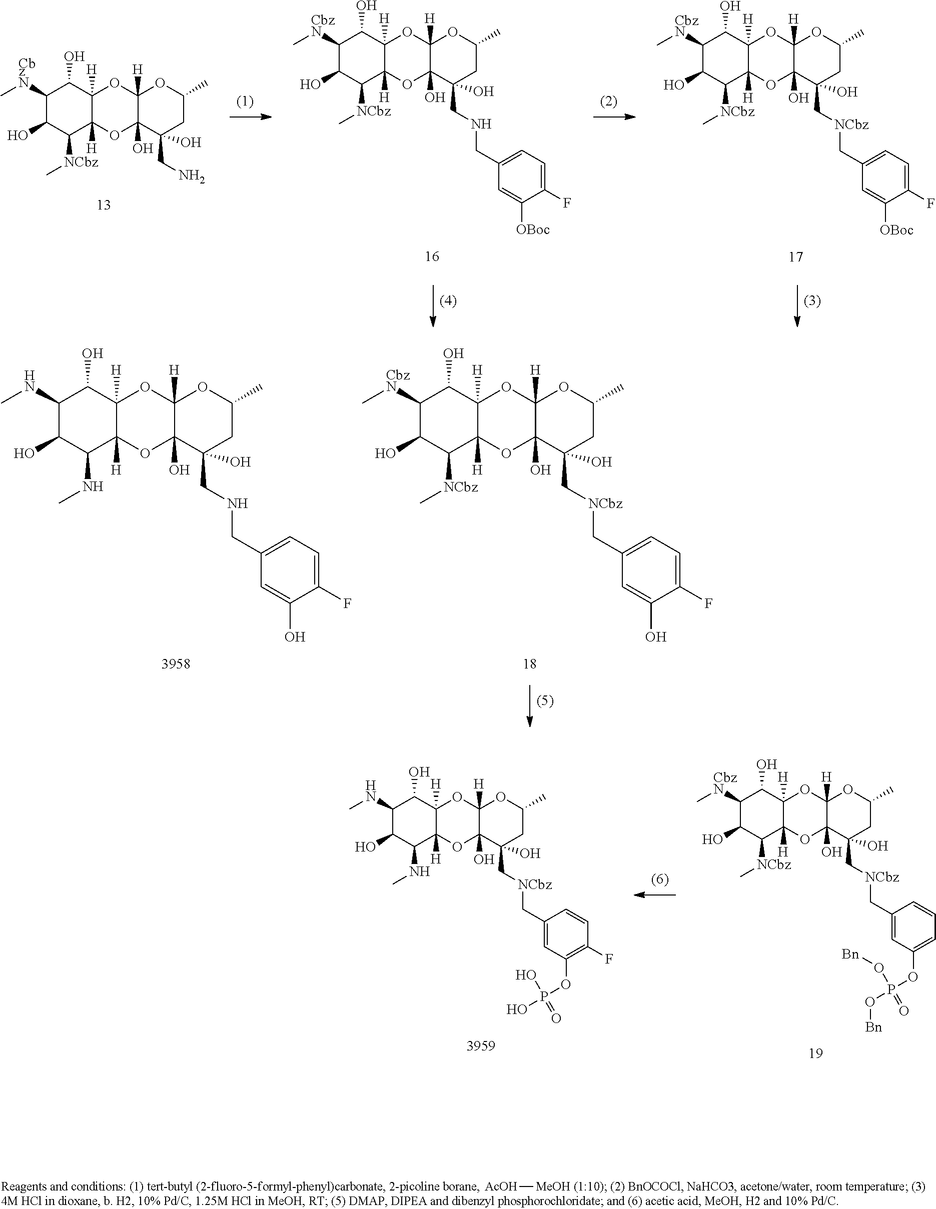

Synthesis of Compound 13: The compound was prepared as described herein above as intermediate during the preparation of (2R, 4R, 4aS, 5aR, 6S, 7S, 8R, 9S, 9aR, 10aS)-4-(((4-hydroxyphenethypamino)methyl)-2-methyl-6,8-bis(methylarnino)octahydro-2H-benzo[b]pyrano[2,3-e][1,4]dioxine-4,4a,7,9(10aH)-tetraol and 4-(2-((((2R, 4R, 4aS, 5aR, 6S, 7S, 8R, 9S, 9aR, 10aS)-4,4a,7,9-tetrahydroxy-2-methyl-6,8-bis(methylamino)decahydro-2H-benzo [b]pyrano[2,3-e][1,4]dioxin-4-yl)methyl)amino)ethyl)phenyl dihydrogen phosphate.

Synthesis of Compound 16: Compound 13 (394 mg, 0.624 mmol) was dissolved into 2 M acetic acid in methanol (10 mL). Tert-butyl (2-fluoro-5-formylphenyl) carbonate (150mg, 0.624 mmol) and Z-picoline borane (46.8mg, 0.437 mmol) were added to the solution of compound 13 in acetic acidlmethanol. The resulting solution was stirred at room temperature overnight. Methanol was removed and the residue partitioned between EA and water; and then the the aqueous layer was extracted with EA (2×10 mL). The combined organic layers were dried with Na₂SO₄ and concentrated under reduced pressure to afford the target compound, 16 (440 mg; 82%).

Synthesis of Compound 3958: Compound 16 (200 mg, C.234 mmol) was dissolved in 4 M HCl in dioxane and stirred for half an hour. The solvent was removed and then the residue was mixed with 10% Pd/C (30 mg) in 1.25 M HCl in MeOH (10 mL), The mixture was allowed to react at room temperature overnight. Pd/C was removed by filtration; and then the filtrate was dried under vacuum to yield the target compound, 3958 (124 mg; 88%). ¹H NMR (400 MHz, Deuterium Oxide) δ 7.19 (td, J=8.4, 11.0 Hz, 1H), 7.08 (ddd, J=2.2, 5.5, 8.2 Hz, 1H), 6.96 (tdd, J=2.2, 4.2, 8.4 Hz, 1H), 4.72 (s, 1H), 4.28-4.11 (m, 3H), 3.93 (dt, J=9.9, 30.0 Hz, 2H), 3.75-3.61 (m, 1H), 3.53-3.41 (m, 1H), 3.33 (d, J=13.6 Hz, 1H), 3.22-3.06 (m, 2H), 2.76 (d, J=1.9 Hz, 6H), 1.89-1.60 (m, 2H), 1.16 (d, J=6.1 Hz, 3H). ESI-MS [M+H]⁺, m/z 488.

Synthesis of Compound 17: Compound 16 (440 mg, 0.514 mmol), sodium bicarbonate (64.8mg, 0,771) and benzyl carbonochloridate (132mg, 0,771mmol) were dissovled in acetone and water. After an hour, the reaction mixture was partitioned between EA and water; and the the aqueous layer was extracted with EA (2×10 mL). The combined organic layers were dried over Na₂SO₄ and concentrated under reduced pressure, followed by purification using column chromatography to afford the target compound, 17 (155 mg, 30.5%).

Synthesis of Compound 19: To a stirred solution of trichloroisocyanuric acid (77 mg, 0.33 mmol) in dry acetonitrile (6 mL) at room temperature was added in a single portion of diisopropyl phosphite, (0,262 g; 1.0 mmol). The resulting mixture was stirred at room temperature until cyanuric acid precipitated from the reaction mixture. The reaction mixture was allowed to settle prior to decanting the clear solution (1.2 mL) into a round bottom flask for next step. Compound 17 (155mg) was dissolved in 4M HCl in dioxane and stirred for half an hour. The solvent was removed and then the residue was added to the acetonitrile solution above, followed by addition of N, N-diisopropylethylarnine (40,4 mg, C.312 mmol) and N,N-dimethylpyridin-4-amine (1.908 mg, 0.016 mmol), The mixture was stirred at room temperature and monitored by UPLC. After about 1 h, the reaction mixture was partitioned between EA and water. The organic layer was washed with brine and dried over Na₂SO₄. After removing solvent, the residue was purified by column chromatography (EA/MeOH) to afford the target compound, 19 (80 mg; 44.5%).

Synthesis of Compound 3959: Compound 19 (80 mg, 0.070 mmol) was mixed with 10% Pd/C (10mg) in acetic acid (500 mg) in MeOH (5 mL), The mixture was allowed to react at room temperature overnight. Pd/C was removed by filtration and then the filtrate was dried under vacuum to yield the target compound, 3959 (35 mg; 89%). ¹H NMR (400 MHz, Deuterium Oxide) δ 7.51 (d, J=7.7 Hz, 1H), 7.15 (dd, J=8.4, 10.7 Hz, 1H), 7.12-6.91 (m, 1H), 4.67-4.55 (m, 1H), 4.24 (d, J=13.3 Hz, 1H), 4.16-4.02 (m, 2H), 3.89 (h, J=9.3, 9.8 Hz, 2H), 3.77-3.65 (m, 1H), 3.39-3.01 (m, 4H), 2.70 (s, 3H), 2.59 (s, 3H), 1.81-1.62 (m, 2H), 1.18-1.10 (m, 3H). ESI-MS [M+H]+, m/z 568.

### 6. 2-(3-fluoro-5-hydroxypyridin-2-yl)-N-((2R, 4R, 4aS, 5aR, 6S, 7S, 8R, 9S, 9aR, 10aS)-4a,7,9-trihydroxy-2-methyl-6,8-bis(methylamino)decahydro-2H-benzo[b]pyrano[2,3-e][1,4]dioxin-4-yl)acetamide and 5-fluoro-6-(2-oxo-2-(((2R, 4R, 4aS, 5aR, 6S, 7S, 8R, 9S, 9aR, 10aS)-4a,7,9-trihydroxy-2-methyl-6,8-bis(methylamino)decahydro-2H-benzo[b]pyrano[2,3-e][1,4]dioxin-4-yl)amino) ethyl)pyridin-3-yl dihydrogen phosphate (Compounds 4065 and 4066, respectively)

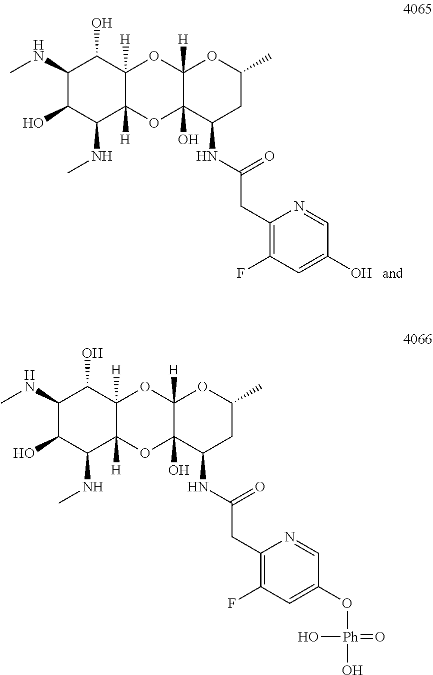

The synthetic scheme to prepare the above-referenced compounds starting from compound 2 is shown immediately below.

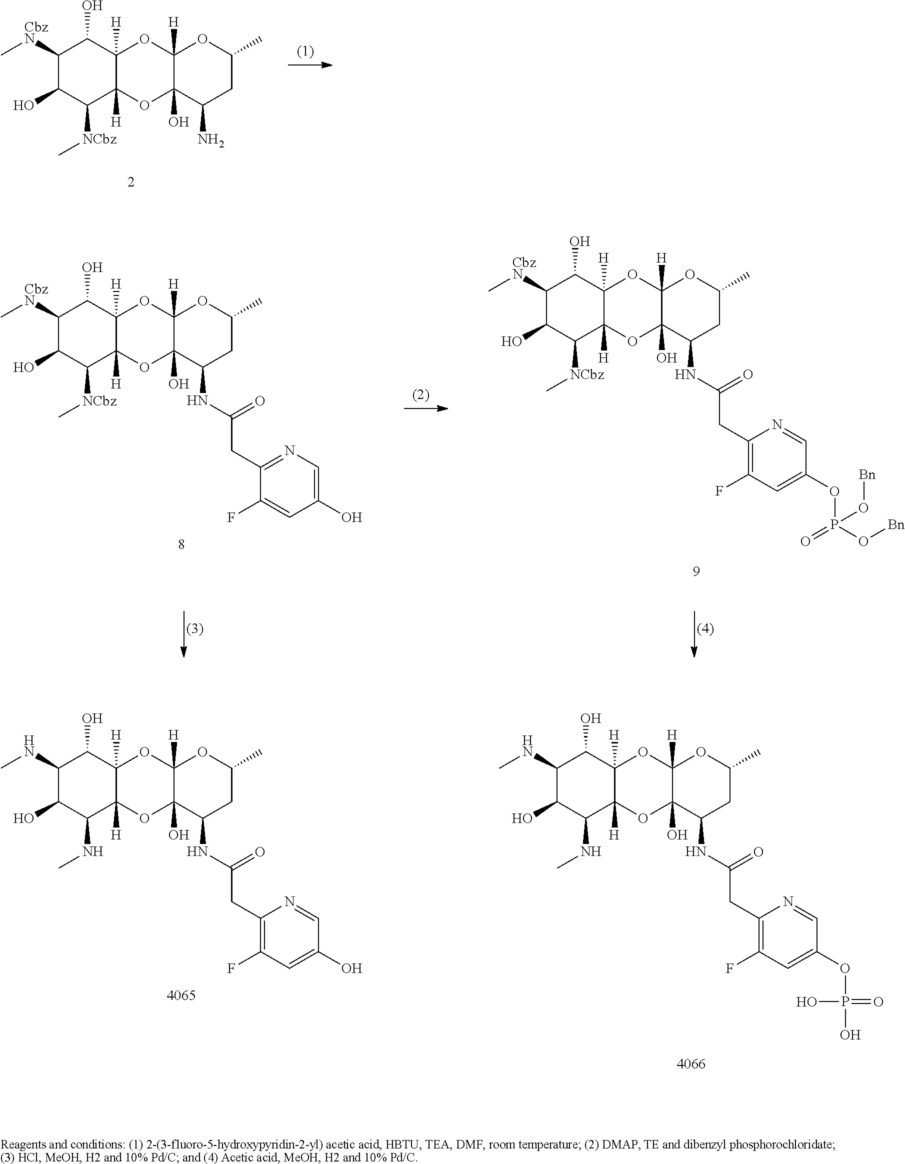

Synthesis of Compound 2: The compound was prepared as described herein above as intermediate during the preparation of 2-(5-hydroxypyridin-2-yI)-N-((2R,4R,4aS,5aR,6S,7S,8R,9S,9aR,10aS)-4a,7,9-trihydroxy-2-methyi-6,8-bis(methylamino) decahydro-2H-benzo[b]pyrano[2,3-e][1,4]dioxin-4-yl)acetamide.

Synthesis of Compound 8: 2-(3-fluoro-5-hydroxypyridin-2-yl) acetic acid (309 mg, 1.81 rnmol), compound 2 (1032 mg, 1.72 mrnol), HBTU (685 mg, 1.81 mrnol) and triethylarnine (TEA. 548 nig, 5,42 mmol) were mixed in DMF (20mL). The reaction mixture was quenched by water in 30 min, Then the solution was extracted by EA (50mL) twice, the combined EA layer was washed by brine, dried over Na₂SO₄. After the solvent was removed, the residue was purified by C18 chromatography and compound 8 (1 g, 73%) was got as white solid.

Synthesis of Compound 4065: Compound 8 (120 mg, 0.16 mmol) was mixed with 10% Pd/C (20 mg) in 1.25M HCl in MeOH (10 mL), the mixture was hydrogenated at room temperature for overnight. Pd/C was removed and then the filtrate was dried under vacuum to yield 94 mg (99%) 4065 as white solid. ¹H NMR (400 MHz, Deuterium Oxide) δ 8.12 (d, J=2.4 Hz, 1H), 7.57 (dd, J=10.5, 2.4 Hz, 1H), 5.03 (s, 1H), 4.42 (dd, J=11.1, 9.7 Hz, 1H), 4.25-3.90 (m, 6H), 3.54 (dd, J=11.1, 2.7 Hz, 1H), 3.29 (dd, J=10.2, 2.9 Hz, 1H), 2.85 (s. 3H), 2.85 (s, 3H), 1.92 (ddd, J=15.2, 11.7, 4.0 Hz, 1H), 1.83-1.71 (m, 1H), 1.28 (d, of =6.1 Hz, 3H). ESI-MS [M+H]⁺, m/z 487.

Synthesis of Compound 9: To a stirred solution of trichloroisocyanuric acid (61 mg, 0.26 mmol) in dry acetonitrile (5 mL) at room temperature was added in one portion of diisopropyl phosphite, (0.21 g, 0.8 mmol). The resulting mixture was stirred at room temperature until cyanuricacid precipitated from the reaction mixture. The reaction mixture was settled down before decanning the clear solution to a round bottom flask for next step. Compound 8 (250mg, C.33mmo1) was added to the solution above, followed by N,N-diisopropylethylamine (86mg, 0.66 mmol) and N,N-dimethylpyridin-4-amine (4.1mg, 0.03 mmol). The mixture was stirred at room temperature and monitored by UPLC, after 1 h, the reaction mixture was partitioned between EA and water. The organic layer was washed with brine and dried over Na₂SO₄. After removing solvent, the residue was purified by chromatography (EA/MeOH) and compund 9 (160 mg, 47%) was got.

Synthesis of Compound 4066: Compound 9 (160mg, 0.158mmo1) was mixed with 10% Pd/C (20 mg) and acetic acid (100 mg) in MeOH (10 mL), the mixture was hydrogenated at room temperature for overnight. Pd/C was removed and then the filtrate was dried under vacuum to yield 46 mg (52%) 4066 as white solid, ¹H NMR (400 MHz, Deuterium Oxide) δ 8.13 (s, 1H), 7.46 (d, J=10.6 Hz, 1H), 4.93 (d, J=4.0 Hz, 1H), 4.33 (t, J=10.3 Hz, 1H), 4.15-3.76 (m, 6H), 3.42 (dd, J=11.2, 2.8 Hz, 1H), 3.18 (dd, J=9.9, 2.7 Hz, 1H), 3,04-2.85 (m, 1H), 2.75 (s, 3H), 274 (s, 3H), 1.79 (dd, J=11.3, 3.8 Hz, 1H), 1.75-1,66 (m, 1H), 1.19 (d, J=6.1 Hz, 3H). ESI-MS [M+H]⁺, m/z 567.

### 7. 2-(5-(4-hydroxyphenyl)pyridin-2-yl)-N-((2R, 4R, 4aS, 5aR, 6S, 7S, 8R, 9S, 9aR, 10aS)-4a,7,9-trihydroxy-2-methyl-6,8-bis(methylamino)decahydro-2H-benzo[b]pyrano[2,3-e][1,4]dioxin-4-yl)acetamide and 4-(6-(2-oxo-2-(((2R, 4R, 4aS, 5aR, 6S, 7S, 8R, 9S, 9aR, 10aS)-4a,7,9-trihydroxy-2-methyl-6,8-bis(methyl-amino)decahydro-2H-benzo[b]pyrano[2,3-e][1,4]dioxin-4-yl)amino)ethyl) pyridin-3-yl)phenyl dihydrogen phosphate (Compounds 4072 and 4074, respectively)

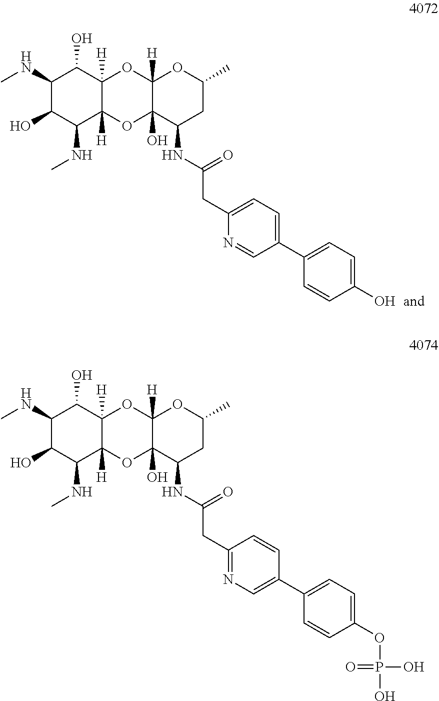 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1ccc(-c2ccc(O)cc2)cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1ccc(-c2ccc(OP(=O)(O)O)cc2)cn1]

The synthetic scheme to prepare the above-referenced compounds starting from compound 2 is shown immediately below.

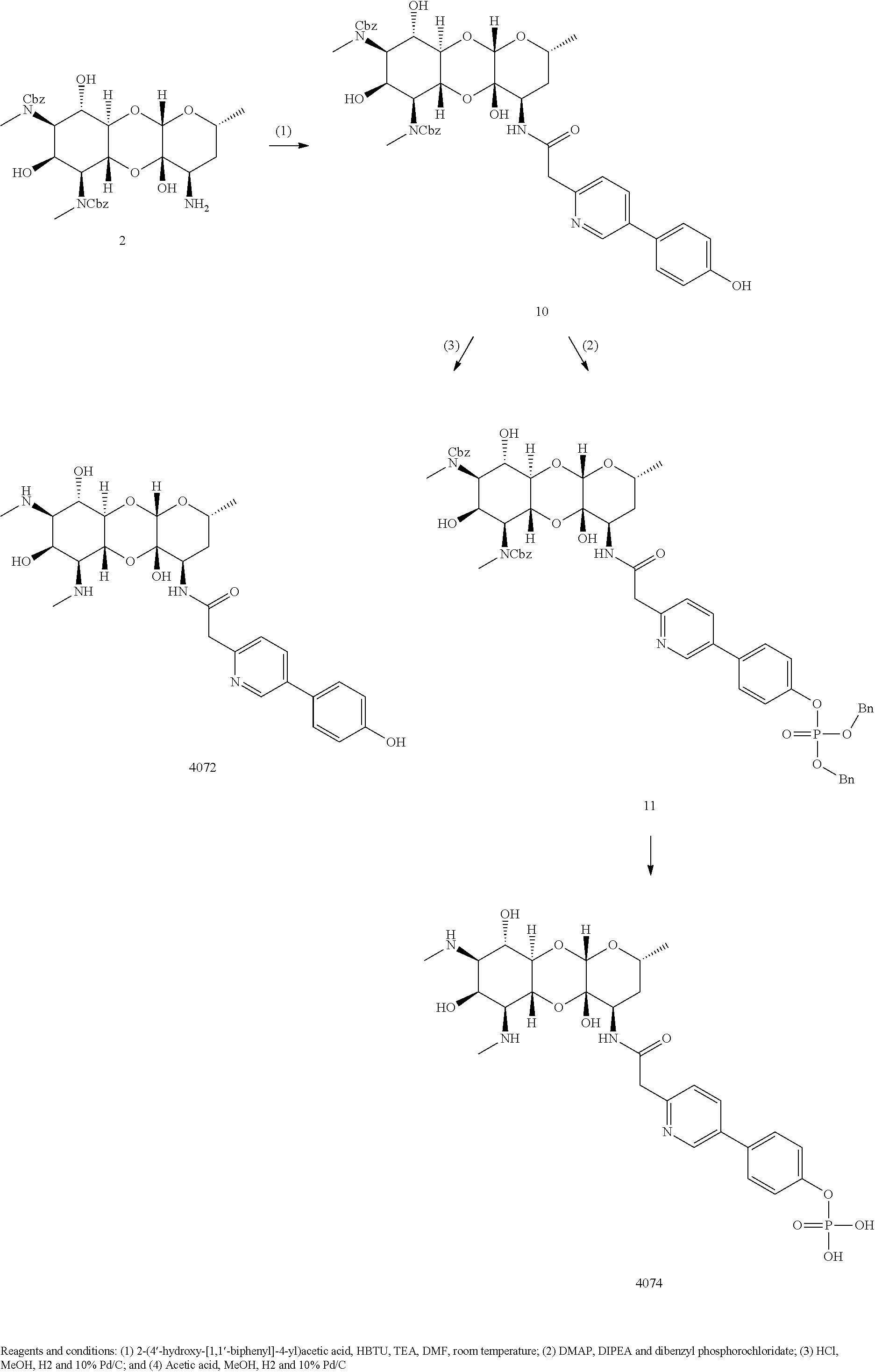

Synthesis of Compound 2: The compound was prepared as described herein above as intermediate during the preparation of 2-(5-hydroxypyridin-2-yl)-N-((2R,4R,4aS,5aR,6S,7S,8R,9S,9aR,10aS)-4a,7,9-trihydroxy-2-methyl-6,8-bis(methylamino) decahydro-2H-benzo[b]pyrano[2,3-e][1,4]dioxin-4-yl)acetamide.

Synthesis of Compound 10: 2-(4′-hydroxy-[1,1′-biphenyl]-4-yl)ace c acid (124 mg, 0.49 mmol), compound 2 (265 mg, 0.441 mmol), HBTU (186 mg, 0.49 mmol) and triethylamine (TEA, 99 mg, 0.98 mmol) were mixed in DMF (10 mL). The reaction mixture was quenched by water after 30 min. The solution was then extracted with EA (25 mL×2). The combined EA layer was washed with brine and then dried over Na₂SO₄. After the solvent was removed, the residue was purified by column chromatography and the target compound, 10, was obtained as a white solid (230 mg; 57.7%).

Synthesis of Compound 4072: Compound 10 (130 mg, 0.16 mmol) was mixed with 10% Pd/C (20 mg) in 1.25 M HCl in MeOH (10 mL). The mixture was allowed to react at room temperature overnight. Pd/C was removed by filtration, and then the filtrate was dried under vacuum to yield the target compound, 4072, (100 mg; 96%) as a white solid. ¹H NMR (400 MHz, Deuterium Oxide) δ 8.94 (d, J=2.2 Hz, 1H), 8.66 (dd, J=8.4, 2.2 Hz, 1H), 7.91 (d, J=8.5 Hz, 1H), 7.75-7.66 (m, 2H), 7.18-7.05 (m, 2H), 5.04 (s, 1H), 4.49-4.38 (m, 1H), 4.29-4.21 (m, 1H), 4.20 (d, J=4.5 Hz, 1H), 4.16-3.95 (m, 3H), 3.55 (dd, J=11.1, 2.7 Hz, 1H), 3.37 (s, 1H), 3.32-3.26 (m, 1H), 2,85 (d, J=1.4 Hz, 6H), 2.01-1.86 (m, 1H), 1,81 (d, J=14.5 Hz, 1H), 1.29 (d, J=6.2 Hz, 3H). ESI-MS [M+H]⁺, m/z 545.

Synthesis of Compound 11: To a stirred solution of trichloroisocyanuric acid (61 mg, 0.264 mmol) in dry acetonitrile (6 mL) at room temperature was added in a single portion of diisopropyl phosphite, (0.21 g; 0.8 mmol). The resulting mixture was stirred at room temperature until cyanuricacid precipitated from the reaction mixture. The reaction mixture was allowed to settle prior to decanting the clear solution (3 mL) into a round bottom flask for use in the next step. Compound 10 (230 mg, 0.283 mmol) was added to the preceding solution, followed by addition of N, N-diisopropylethylamine (73.1 mg, 0,566 mmol) and N,N-dimethylpyridin-4-amine (3.46 mg, 0.028 mmol). The mixture was stirred at room temperature and monitored by UPLC. After 1 h, the reaction mixture was partitioned between EA and water. The organic layer was washed with brine and dried over Na₂SO₄. After removing solvent, the residue was purified by column chromatography (EA/MeOH) to obtain target compound. 11 (80 mg, 26%).

Synthesis of Compound 4074: Compound 11 (40 mg, 0.037 mmol) was mixed with 10% Pd/C (10 mg) and acetic acid (500 mg) in MeOH (5 mL). The mixture was allowed to react at room temperature overnight. Pd/C was removed by filtration and then the filtrate was dried under vacuum to provided the target compound, 4074 (20 mg; 86%). ¹H NMR (400 MHz, Deuterium Oxide) δ 8.73 (s, 1H), 8.08 (d, J=8.2 Hz, 1H), 7.65 (d, J=8.2 Hz, 2H), 7.46 (d, J=8.0 Hz, 1H), 7.34 (d, J=8.2 Hz, 2H), 5.01 (s, 1H), 4.41 (t, J=10.2 Hz, 1H), 4.32-3.82 (m, 4H), 3.72-3.52 (m, 1H), 3.44 (d, J=11.0 Hz, 1H), 3.22 (d, J=9.6 Hz, 1H), 2.82 (s, 3H), 2.80 (s, 3H), 1.88 (d, J=13.9 Hz, 1H), 1.85-1.76 (m, 1H), 1.27 (d, 6.2 Hz, 3H). ESI-MS [M+H]⁺, m/z 625.

### 8. SAMPLE PREPARATION AND LC-MS/MS QUANTITATION

LC-MS Method 1. Quantitative determination of the amount of a disclosed compound present in blood, plasma, or urinary can be determined by LC-MS/MS methods. Briefly, a suitable method involves use of calibrants (0.976, 1.95, 3.91, 7.81, 15.63, 31.25, 62.5, 125, 250, 500, 1000 ng/mL) were in sample matrix (e.g., rat plasma, blood or urine). A separate solution of disclosed compound (independent of calibrants) is used to prepare quality controls. Appropriate volumes of working solutions are added to the sample matrix to yield the final concentrations of 1, 50, 1000 ng/mL. Linearity for calibrants in duplicate is assessed by subjecting the spiked concentrations and the respective peak areas to least-square linear regression analysis with and without intercepts, and a weighted least-square regression (1/x or 1/x²). The lowest limit of quantitation (LLOQ) in the standard curve measured with acceptable accuracy and precision for a disclosed compound from rat plasma has been previously established as 0.976 ng/mL for a representative compound, 1810. All samples are precipitated by the addition of 4-volumes (100 μL) of IS, 1369 (10 ng/mL) in methanol to a volume (25 μL) of plasma/urine test sample. The samples are vortexed (Fisher scientific, USA) for 30 sec and centrifuged at 10,000×g for 10 min at 4° C. and the supernatants are collected for LC-MS/MS analysis. Chromatographic separation is carried out using a Shimadzu Nexera XR (LC-20ADXR) liquid chromatograph (Shimadzu Corporation, USA) consisting of two pumps, online degasser, system controller and an auto sampler. Mobile phase consisting of a) water with 1.6% nonafluoropentanoic acid and 0.7% formic acid b) 90% acetonitrile with 0.8% nonafluoropentanoic acid and 0.35% formic acid can be used at a flow rate of 0.5 rnLirnin in gradient mode. A Waters® Symmetry ® 3.5μ C8, 50×2.1 mm column (Waters, Milford, Mass.) is used for the separation. Samples (5 μL) are injected on column and the eluate is provided directly to an API 5500 triple quadruple mass spectrometer (Applied Biosystems, Foster City, Calif.) equipped with a turbospray ion source operated in the positive ion mode. The optimized multiple reactions monitoring (MRM) conditions for 3408 (549.2A207.1), 1810 (469.2^207.1) and IS (418.3^207.1) are monitored with a declustering potential of 45 V and collision energy of 25 eV. Data are acquired and processed with Analyst software version 1.6.2 (Applied Biosystems, Foster City, Calif.).

LC-MS Method 2. Chromatographic separation was performed on an Acquity UPLC BEH C18 1.7 μm, 2.1×50 mm column (Waters Corporation, Milford, Mass.) or a Luna Omega 1.6 μm C18, 2.1×50 mm column (Phenomenex, Torrance, Calif.) using an Acquity ultra performance liquid chromatography system. Data were acquired using Masslynx v. 4.1 and analyzed using the Openlynx software suite. This was coupled to an Acquity photodiode array detector, which acquired UV data from 210-400 nm. The flow was then split, with half directed to an evaporative light scattering detector (ELSD) and half to an SQ mass spectrometer. The total flow rate was 1 mL/min. The sample injection volume was 10 μL. The UPLC column was maintained at 60° C. and the gradient program with either basic mobile phase (A1, 10 mM ammonium bicarbonate in MilliQ H₂O; B1, 0.1% formic acid in acetonitrile) or formic mobile phase (A1, 0.1% formic acid in MilliQ H₂O; B1, 0.1% formic acid in acetonitrile) based on the compounds' physical and chemical properties. The mass spectrometer was operated in positive-ion mode with electrospray ionization. The conditions were as follows; capillary voltage 3.4 kV, cone voltage 30 V, source temperature 130° C., desolvation temperature 400° C., desolvation gas 800 L/hr, cone gas 100 L/hr. A full scan range from m/z=110-1000 in 0.2 s was used to acquire MS data. The ELSD-drift tube temperature was set at 52° C.

### 9. IN VITRO ACTIVITY OF DISCLOSED COMPOUNDS

Minimum inhibitory concentrations (MICs) were determined using the microbroth dilution method according to Clinical Laboratory Standards Institute (CLSI; National, C. F. C. L. S., Methods for Dilution Antimicrobial Susceptibility Tests for Bacteria Grow aerobically-Seventh Edition: Approved Standard M7-A7, CLSI, Wayne, Pa., USA, 2008) and were read by visual inspection. Two fold serial dilutions of antibiotic in 100 μL of the appropriate broth media were first prepared in 96-well round bottom microtiter plates (Nalge Nunc International, Rochester, N.Y., USA). An equivalent volume (100 μL) of bacterial broth inocula containing approximately 10⁵ bacterial cfu/mL was added to each well to give final concentrations of drug starting at 200 μg/mL and the plates were incubated aerobically at 37° C. M. tuberculosis microtiter plates were incubated for 7 days and all other strains were incubated overnight. After incubations, in all cases the MIC was recorded as the lowest concentration of drug that prevented bacterial growth.

In vitro activity was also assessed using a ribosomal inhibition assay. Briefly, luciferase-based protein synthesis inhibition assays were performed in translation reaction as described previously (Bruell, et al. Biochemistry. 2008 47 (34), 8828-8839), using purified Mycobacterium smegmatis 70S bacterial ribosomes and firefly luciferase mRNA prepared in vitro using T7 RNA polymerase. IC50 values represent the drug concentration that inhibits luciferase activity by 50%.

The above two assays were used to assess the activity of compound 1810, and the corresponding disclosed prodrug, compound 3408 (see Table 1 below). The data show that the prodrug form is less active in these in vitro assay systems. Without wishing to be bound by a particular theory, it is believed that under the conditions of these assays, the prodrug form remains intact and is not converted to the parent drug. The data suggest that the phosphate moiety, when present, decreases in vitro activity.

TABLE 1

| | MIC (H37Rv) | IC₅₀ (M. smegmatis) |
|---|---|---|
| Compound | (μg/mL) | (μg/mL) |
| 1810 | 0.8 | 0.84 |
| 3408 | 100 | 6.70 |

### 10. PLASMA AND METABOLIC STABILITY OF DISCLOSED COMPOUNDS

The stability of compounds to human liver microsomal degradation, i.e., metabolic stability, was determined as described previously (see North, E. J., et al, Bioorg. Med. Chem. (2013) 21(9):2587-2599) and the rate of degradation was monitored by LC-MS analysis using multiple sampled time points as described herein above in LC-MS Method 2. Metabolic stability was evaluated via the half-life from least squares fit of the concentration remaining at the time points based on first-order kinetics.

Plasma stability was determined after incubation in human plasma for various incubation time, i.e., incubation times of 0, 3, 24, and 48 hour were used. DMSO stock solutions of test compounds were prepared at 10 mM concentration. 1.8 μL of each test compound was each added directly to 1.8 mL of human plasma to get the final concentration of 10 μM. This compound/matrix was mixed and 70 μL was transferred to 4 time points plates (each in triplicate wells). For the Time 0 plate, 210 μL of cold acetonitrile with internal standard (4 μg/ml warfarin) was added to each well and no incubation needed. For the plates corresponding to the other three time points, the plate was incubated at 37° C. for required time, followed by quenching of the reaction by adding 210 μL of cold acetonitrile or methanol with internal standard (4 μg/ml warfarin) to each well. All of the plates were sealed, mixed well, and centrifuged at 4000 rpm for 20 min, The supernatants (120 μL) were transferred to analytical plates for analysis by LC-MS as described herein above in LC-MS Method 2. The plasma stability was evaluated via the half-life from least-squares fit of the multiple time points based on first-order kinetics.

Using the foregoing methods, several representative disclosed compounds were assessed for their plasma and metabolic stability, and the data are provided in Table 2 below. The data show that the phosphate (—OPO₃H₂) derivatives were rapidly converted in plasma to their parent, whereas the alkyl phosphate (—OPO₃Et₂) derivatives were more stable in plasma. The compounds that were test demonstrated limited metabolic stability, ranging from <5 minutes to several hours.

TABLE 2

| | Plasma Stability (Human) | | | Metabolic Stability (Human) | | |
|---|---|---|---|---|---|---|
| Compound | t½ | | STD | t½ | | STD |
| 3408 | <5 | min | — | <5 | min | — |
| 3906 | 19.5 | hr | 48.63 | 3.61 | hr | 0.58 |
| 3959 | — | | — | 1.84 | hr | 0.59 |
| 4074 | <5 | min | — | 2.98 | hr | 0.41 |

### 11. MURINE MODEL OF ACUTE TB INFECTION OF DISCLOSED COMPOUNDS

All infections were performed at Colorado State University in a certified ABSL3 facility in accordance to guidelines of the Colorado State University Institutional Animal Care and Use Committee. Six- to 8-week-old female specific-pathogen-free BALB/c mice were purchased from Charles River Laboratories (Wilmington, Mass.). Mice were infected with M. tuberculosis Erdman (TMCC107) via a low-dose aerosol exposure in a Glas-Col aerosol generation device (Glas-Col Inc., Terre Haute, Ind.).¹³ Each treatment group consisted of six mice. Treatment was started at 7 d post-aerosol infection and continued for 12 consecutive days. Drugs were administered by subcutaneous injection or oral gavage in a 0.2 mL volume. Spectinamides 1810 or 3408 were formulated in Plasma-Lyte A pH 7.4 and administered daily by subcutaneous injection at 400 mg/kg. Rifampicin was prepared in sterile water and administered daily by oral gavage at 10 mg/kg. For endpoint analysis, mice were euthanized one day following the end of treatment, and the lungs collected. The left lung lobe was homogenized for enumeration of CFU by plating dilutions of the organ homogenates on Middlebrook 7H11 medium supplemented 10% v/v OADC, 0.03 mg/mL cycloheximide and 0.05 mg/mL carbenicillin. The data were expressed as mean log10 CFU±the standard error of the mean (SEM) for each group. Statistical analysis was by one analysis of variance with Dunnet's post-test to control for multiple comparisons (SigmaPlot, San Jose, Calif.). Values were considered significant at the 95% confidence level.

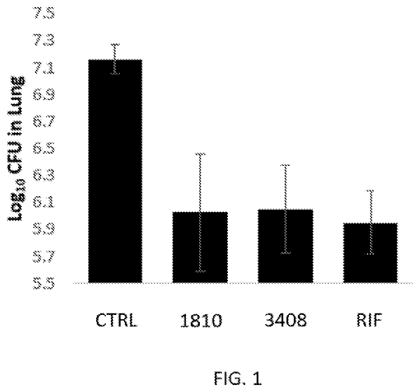

The data obtained in this study are shown in FIG. 1. The data show that compound 1810, and the disclosed prodrug form, compound 3408, were similarly active in this in vivo model. The in vivo data are in contrast to the in vitro data described above. As discussed above, without wishing to be bound by a particular theory, it is believed that the phosphate moiety of the prodrug needs to be removed in order to achieve maximum efficacy. It is likely that in the in vitro assay the phosphate moiety of the prodrug remains intact, whereas upon in vivo administration, it is labile and the compound is rapidly converted to the parent drug form, presurneably via hydrolysis of the phosphate group via esterases present in blood.

### 12. MAXIMUM TOLERATED DOSE STUDY IN RAT OF DISCLOSED COMPOUNDS

Several compounds were assessed in single ascending maximum tolerated studies. The compounds assessed were 1810; 3408, a prodrug form of compound 1810; 1599, a chloro homologue of compound 1810; and gentamicin, an antimicrobial associated with ototoxicity. The study was carried out in female Sprague-Dawley rats (n=3 per group). All the drug solutions were prepared in water and PLASMA-LYTE A (50:50; PLASMALYTE A was obtained from Baxter Healthcare Corporation, Deerfield, Ill.). Each group of rats were fasted overnight with access to water aJ libitum and a fixed volume (2 mL) of drug-containing solution was administered via the jugular vein catheter. Food was withheld for 3 h post dosing. Different phases of the study were classified on five different speeds of i.v, administration; Phase 1: 5 sec, Phase 2: 30 sec, Phase 3: 5 min, Phase 4: 15 min, Phase 5: 30 min. In the first and second phases, a set of predefined dose levels: 10, 25, 50, 75, 100, 150, 300, 350, 400, 450 and 500 mg/kg. Escalation to the next higher dose was conditional upon absence of signs of acute toxicity following the immediate lower dose over a 24 h period. All animals were monitored by visual inspection focused on focus on changes in skin, fur, eyes, and also respiratory, circulatory, autonomic and central nervous systems, along with somatomotor activity and behavior pattern, including tremors, convulsions, salivation, diarrhea, lethargy, sleep and coma. A dose level was deemed an MTD if any signs of acute toxicity were observed and that dose was chosen for the second phase of the study where the process was repeated to determine a toxic dose. For third, fourth and fifth phases, dosing solution was infused by a Instech tethering system (Instech Laboratories Inc., Plymouth Meeting, Pa.) where the animal is freely moving within its cage, the catheter connection is held in place by a vascular access harness and a swivel system, and the drug administration is performed via a Harvard Apparatus programmable syringe pump. After dosing, the tether was removed immediately after completion of the infusion and the catheter was flushed with (100 μL) PlasmaLyte A. Surviving animals from the previous dose with no signs of toxicity were re-used in either phases of these studies upon allowing a one day drug washout period.

The data from this study are summarized in Table 3 below. These data show that the parent compound, 1810, exhibited a maximum tolerated dose in these studies of about 200-350 mg/kg (for IV bolus and 5 minute IV infusion), unless administered via a 15 minute IV infusion. The chloro homologue of 1810, i.e., compound 1599, demonstrated a lower maximum tolerated dose of about 75-100 mg/kg by all administration routes, except when administered via a 30 minute infusion. Gentamicin showed a maximum tolerated dose profile similar to compound 1599. In contrast, compound 3408, a prodrug form of compound 1810, was very well tolerated and in these studies demonstrated a maximum tolerated does of >500 mg/kg when administered by the most rapid route, i.e., a 5 second IV bolus.

TABLE 3

| | MTD (mg/kg) | | | |
|---|---|---|---|---|
| Administration | 1599 | 1810 | 3408 | Gentamicin |
| 5 second IV bolus | 75 | 200 | >500 | 100 |
| 30 second IV bolus | 100 | 200 | — | 100 |
| 1 minute IV bolus | 100 | — | — | — |
| 5 minute IV infusion | 100 | 350 | — | 200 |
| 15 minute IV infusion | 150 | >500 | — | 300 |
| 30 minute IV infusion | 300 | — | — | 400 |

### 13. PHARMACOKINETIC STUDY IN RAT

Pharmacokinetic assessment of the disclosed compounds can be determined in a rat model as described herein. Briefly, catheterized male Sprague-Dawley rats from Harlan Bioscience (Indianapolis, Ind.), weighing 200-250 g, are kept at a 12 h light/dark cycle with access to food and water ad libitum. The procedures are approved by the Institutional Animal Care and Use Committee of the University of Tennessee Health Science Center. Formulations of a disclosed compound are prepared in water (50%) and PlasniaLyte A (50%). Test compound can be administered either via intravenous route (10 mg/kg) via femoral vein catheter with the study conducted using a group of 6 rats. Blood (250 μL) was collected via the jugular vein catheter at 0.08, 0.25, 0.5, 0.75, 1, 1.5, 2, 4, 6, 8, 10, 24 and 48 h after drug administration. Plasma was immediately separated by centrifugation (6,000×g for 10 min at 4° C.) and stored at −70° C. until analysis. The plasma concentration was determined as described herein above in LC-MS Method 1, and plasma concentration-time courses were fitted to a non-compartmental model using Phoenix WinNonlin 7.0 (Certara L. P., Princeton, NJ). Area under the concentration-time profile curve (AUC) was calculated by the log trapezoidal rule by log-linear regression and extrapolated to infinity by addition of the value Ct/λ_{Z}, where Ct is the concentration at the last time point, and λ_{Z} the terminal slope (determined by linear regression) of the log concentration-time curve. The terms Cₘₐₓ,ₐ, and tₘₐₓ represent the maximum concentration achieved and the time to maximum concentration respectively and were determined from the concentration-time profiles.

In the pharmacokinetic study, compound 1810 or 3408 were dosed was via intravenous administration using the above-described method. Due to the rapid conversion in plasma of 3408 in plasma to compound 1810, plasma samples for each study (dosing with either 1810 or 3408) were analyzed for the level of compound 1810, The data from a representative study are given below in Table 4. The data show that the prodrug, compound 3408, is rapidly converted in vivo to the parent drug form, compound 1810, and shows a very similar pharmacokinetic profile to non-prodrug parent.

TABLE 4

| | AUC | Cmax | CL | Vss | t½ |
|---|---|---|---|---|---|
| Compound | (h*mg/L) | (mg/L) | (L/h/kg) | (L/kg) | (h) |
| 1810 from | 18.5 ± | 38.9 ± | 0.546 ± | 0.322 ± | 0.390 ± |
| 3408 | 1.95 | 4.77 | 0.0567 | 0.0674 | 0.0392 |
| 1810 | 22.0 ± | 47.0 ± | 0.463 ± | 0.494 ± | 0.392 ± |
| | 3.02 | 6.12 | 0.0738 | 0.308 | 0.0160 |

### 14. PLASMALYTE A STABILITY STUDY

Stability of compound 3408 was determined in a mixture of PlasmaLyte A and milli-Q water (50:50) at room temperature. Briefly, a 10 mg/mL solution of compound 3408 was prepared in triplicate and samples were collected (0, 1, 2, 3, 4, 5, 6 h). These samples were processed by addition of 4-volumes of methanol and quantitated by LC-MS/MS method disclosed herein in LC-MS Method 1. The percentage drug remaining at each hour was calculated with respect to the concetration at the initial time point. The data from a representative experiment is shown below in Table 5. The data show that compound 3408 (10 mg/mL) is stable for at least 6 hours at room temperature in Plasmalyte A and milli-Q water mixture (50:50).

TABLE 5

| Hour | % remaining | STDEV |
|---|---|---|
| 0 | 100 | 0 |
| 1 | 103 | 3.45 |
| 2 | 101 | 1.84 |
| 3 | 105 | 2.09 |
| 4 | 104 | 4.37 |
| 5 | 104 | 4.18 |
| 6 | 104 | 2.29 |

### 15. PROSPECTIVE IN VITRO PLASMA PROTEIN BINDING

Rat plasma protein binding can be determined by equilibrium dialysis using a RED device (Thermo Scientific, Rockford, USA) containing plasma and buffer chambers for dialysis and a base plate. For example, two concentrations (0.5 and 5 mg/L) of a disclosed compound can be prepared in rat plasma, and then an aliquot of 300 μL is added in the plasma chamber in duplicate. A 500 μL aliquot of PBS is added in the buffer chamber for dialysis. Then the base plate is covered with sealing tape and incubated at 37° C. at approximately 100 rpm on an orbital shaker for 4 h to achieve equilibrium. After incubation, 50 μL of each sample is pipetted from the plasma and buffer chambers into separate micro-centrifuge tubes. 50 μL of plasma is added to the buffer samples and an equal volume of PBS to the collected plasma samples, followed by vortexing. Samples are processed by protein precipitation and analyzed for bound and unbound drug concentrations, which are determined using LC-MS/MS as described herein in LC-MS Method 1. Ranitidine (0.5 and 5 mg/L) can be included as a positive control.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A compound having a structure represented by a formula: 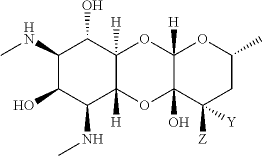
wherein Y is hydrogen or hydroxyl;
wherein Z is —CH₂NH(C1-C3 alkanediyl)Ar¹ or —NH—(C═O) —(C1-C3 alkanediyl) Ar¹ ;
wherein Ar¹ is aryl or heteroaryl substituted with:
a) a R²⁰ group, wherein R²⁰ is selected from (C1-C3)-alkanediyl-OP(O)(OR²¹) (OR²²), —P(O)(OR²¹)(OR²²), —(C1-C3)-alkanediyl-OSO₂OR²¹, —OSO₂OR²¹, —(C1-C3)-alkanediyl-OSO₂R²¹, —OSO₂R²¹, (C1-C3)-alkanediyl OSO₂NR²¹R²²,
OSO₂NR²¹R²², wherein each of R²¹ and R²² is independently selected from hydrogen and C1-C3 alkyl; and
b) 0 to 2 groups independently selected from halo, cyano, hydroxyl, —NH₂, C1-C3 alkyl, C1-C3 haloalkyl, C1-C3 alkoxy, and C1-C3 haloalkoxy;
or a pharmaceutically acceptable salt thereof.

2. The compound according to claim 1, wherein Z is —NH(C═O)(C1-C3 alkanediyl)Ar¹.

3. The compound according to claim 2, wherein Z is NH(C═O)(CH₂)_{q}Ar¹; and wherein q is an integer selected from 0, 1, 2, and 3.

4. The compound according to claim 1, wherein Ar¹ is a structure represented by a formula: 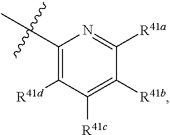
wherein 1 of R^{41a}, R^{41b}, R^{41c}, and R^{41d} is R²⁰;
wherein 1, 2, or 3 of R^{41a}, R^{41b}, R^{41c}, and R^{41d} are independently hydrogen; and,
wherein 0, 1, or 2 of R^{41a}, R^{41b}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

5. The compound of claim 4, wherein 0, 1, or 2 of R^{41a}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

6. The compound of claim 4, wherein R^{40a} is hydrogen or fluoro; and wherein each of R^{41b}, R_{41c}, and R^{41d} is hydrogen.

7. The compound of claim 4, wherein R^{40b} is —(C1-C3)-alkanediyl-OP(O)(OR²¹)(OR²²) or —OP(O)(OR²¹)(OR²²).

8. The compound of claim 8, wherein R^{40b} is OP(O)(OR²¹)(OR²²).

9. The compound of claim 9, wherein each of R²¹ and R²² is independently selected from hydrogen, methyl, and ethyl.

10. The compound of claim 4, wherein R^{40b} is —(C1-C3)-alkanediyl-OSO₂OR²¹ or —OSO₂OR²¹.

11. The compound of claim 11, wherein R^{40b} is—OSO₂OR²¹.

12. The compound of claim 12, wherein R²¹ is selected from hydrogen, methyl, and ethyl.

13. The compound of claim 1, present as: 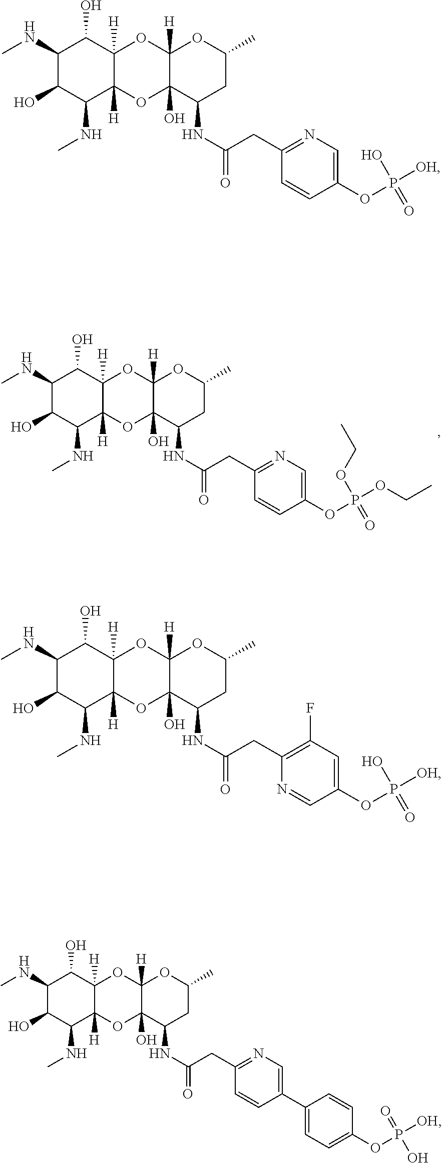 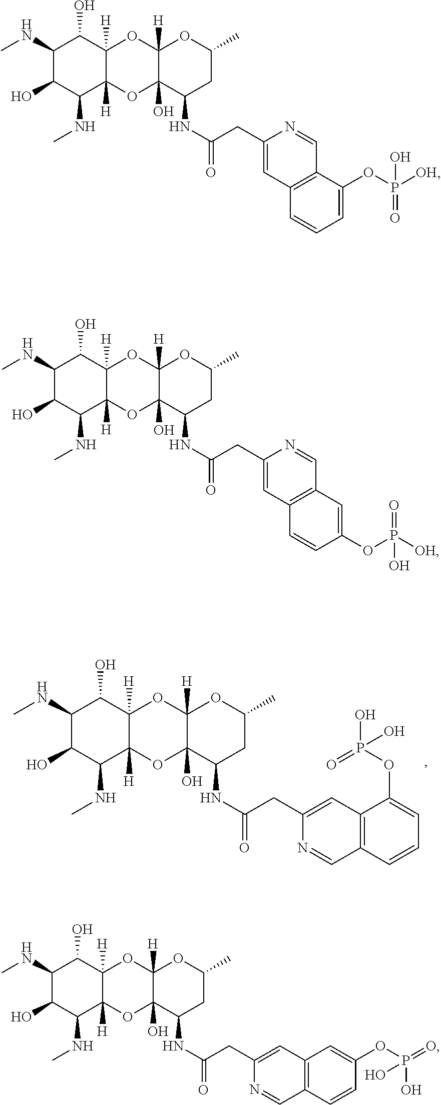 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1cc2cccc(OP(=O)(O)O)c2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2ccc(OP(=O)(O)O)cc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2c(OP(=O)(O)O)cccc2cn1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cc2cc(OP(=O)(O)O)ccc2cn1] 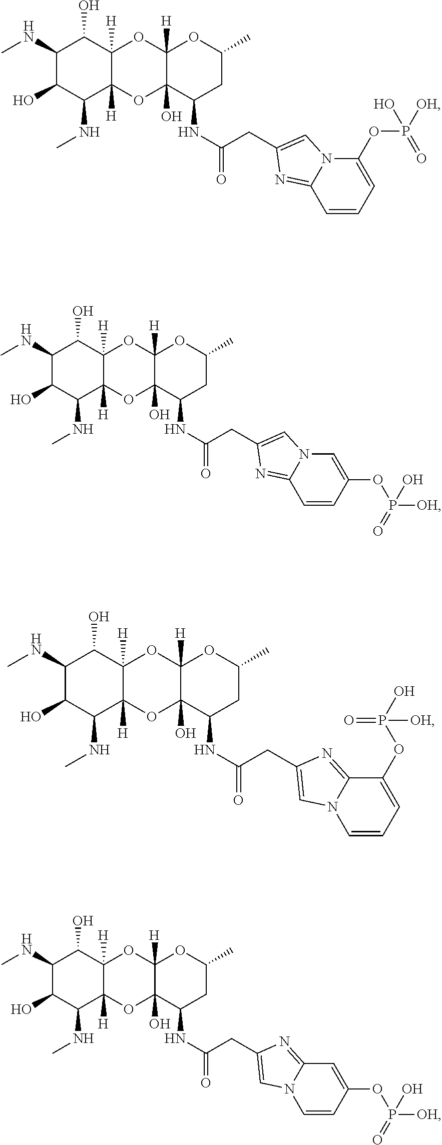 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1cn2c(OP(=O)(O)O)cccc2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2cc(OP(=O)(O)O)ccc2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2cccc(OP(=O)(O)O)c2n1 ; CN[C@@H]1[C@H](O)[C@H](NC)[C@H]2O[C@]3(O)[C@H](O[C@@H]2[C@H]1O)O[C@H](C)C[C@H]3NC(=O)Cc1cn2ccc(OP(=O)(O)O)cc2n1] 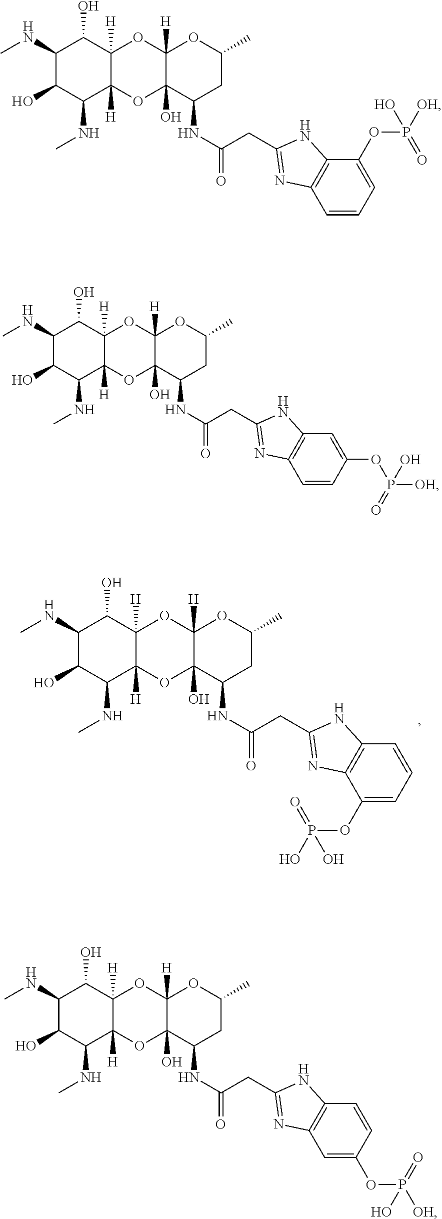 [SMILES: CN[C@@H]1[C@H](O)[C@H](NC)[C@H]2O[C@]3(O)[C@H](O[C@@H]2[C@H]1O)O[C@H](C)C[C@H]3NC(=O)Cc1nc2cccc(OP(=O)(O)O)c2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2ccc(OP(=O)(O)O)cc2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2c(OP(=O)(O)O)cccc2[nH]1 ; CN[C@@H]1[C@H](O)[C@H](NC)[C@H]2O[C@]3(O)[C@H](O[C@@H]2[C@H]1O)O[C@H](C)C[C@H]3NC(=O)Cc1nc2cc(OP(=O)(O)O)ccc2[nH]1] 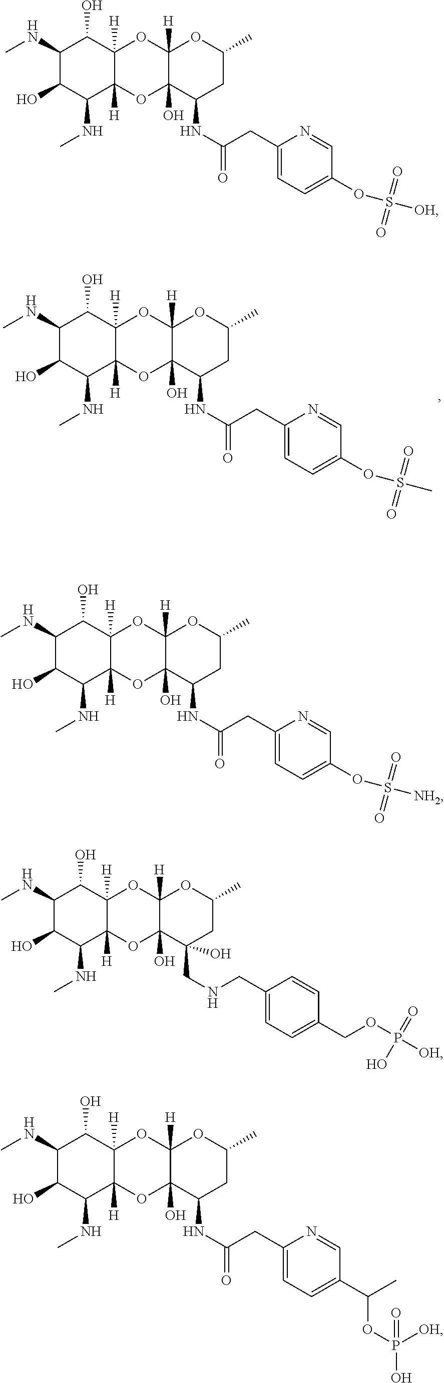 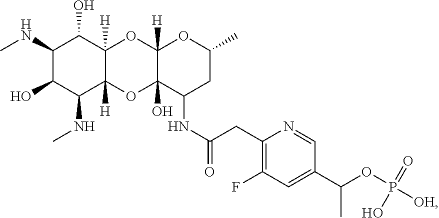 [SMILES: CN[C@@H]1[C@H](O)[C@H](NC)[C@H]2O[C@@]3(O)C(NC(=O)Cc4ncc(C(C)OP(=O)(O)O)cc4F)C[C@@H](C)O[C@H]3O[C@@H]2[C@H]1O]
or a subgroup thereof.

14. A pharmaceutical composition comprising a therapeutically effective amount of a compound of claim 1, or a pharmaceutically acceptable salt thereof, and a pharmaceutically acceptable carrier.

15. The pharmaceutical composition of claim 14, wherein the compound has a structure represented by a formula: 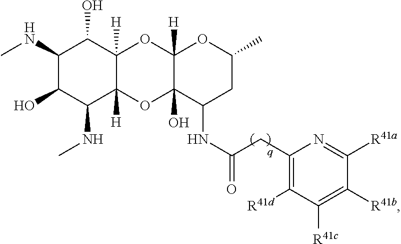
wherein 1 of R^{41a}, R^{41b}, R^{41c}, and R^{41d} is R²⁰;
wherein 1, 2, or 3 of R^{41a}, R^{41b}, R^{41c}, and R^{41d} are independently hydrogen; and,
wherein 0, 1, or 2 of R^{41a}, R^{41b}, R^{41c}, and R^{41d} are independently selected from halo, cyano, hydroxyl, —NH₂, C1-C3 alkyl, C1-C3 haloalkyl, C1-C3 alkoxy, and C1-C3 haloalkoxy.

16. The pharmaceutical composition of claim 14, further comprising a second active agent;
and wherein the second active agent comprises at least one anti-tuberculosis agent.

17. The pharmaceutical composition of claim 16, wherein the anti-tuberculosis agent is selected from amikacin, amoxicillin-clavulanic acid, bedaquiline, capreomycin, ciprofloxacin, clarithromycin, clofazimine, cycloserine, delamanid, ethambutol, ethionamide,gatifloxacin, imipenem, isoniazid, kanamycin, levofloxacin, linezolid, meropenem, moxifloxacin, ofloxacin, para-aminosalicylic acid, pretomanid, pyrazinamide, rifampin, rifapentine, rifabutin, SQ109, streptomycin, sudoterb, terizidone, thiacetazone, viomycin, and combinations thereof.

18. A method for the treatment of an infectious disease in a human subject comprising the step of administering to a subject a therapeutically effective amount of at least one compound of claim 1, or a pharmaceutically acceptable salt thereof.

19. The method of claim 18, wherein the infectious disease is associated with a Mycobacterium sp. infection.

20. The method of claim 19, wherein the Mycobacterium sp. is M. tuberculosis; and wherein the M. tuberculosis is M. tuberculosis complex comprising one or more of M. tuberculosis sensu stricto, M. africanum, M. canetti, M. bovis, M. caprae, M. microti, M. pinnipedii, M. mungi, and M. orygis.
